(19) United States
(12) Patent Application Publication
(10) Pub. No.: US 2026/0265353 A1
(43) Pub. Date: Sep. 10, 2026

(54) DUAL-SPECIFIC BIFUNCTIONAL FUSION PROTEINS FOR UBIQUITIN-MEDIATED DEGRADATION

(71) Applicant: AXELYF EHF., HAFNARF JORDUR (IS)

(72) Inventors: HENG LI, NEW YORK, NY (US); MARIUS S POP, NEW YORK, NY (US); CHRISTOPHER M. WILFONG, NEW YORK, NY (US); JUWINA WIJAYA, NEW YORK, NY (US); KERRY E. BENENATO, NEW YORK, NY (US)

(21) Appl. No.: 19/120,378

(22) PCT Filed: Oct. 13, 2023
(86) PCT No.: PCT/US2023/076886

§ 371 Date: Apr. 11, 2025

Related U.S. Application Data
(60) Provisional application No. 63/379,509, filed on Oct. 14, 2022.

(51) Int. Cl. C07K 16/18; C07K 16/30; C07K 16/32

(52) U.S. Cl. CPC C07K 16/18; C07K 16/30; C07K 16/32; C07K 2317/31

(57) ABSTRACT

Disclosed herein are polypeptides, and dual-specific bifunctional polypeptides that induce degradation of a first and/or second biological target through the cellular proteasome mechanism. The dual-specific bifunctional polypeptides generally involve a first and second component that binds to the biological target(s), and a third component that has ubiquitination function, where the binding of the biological target(s) by the first and/or second component promotes ubiquitination of the biological target(s) by the third component, thereby labeling the biological target(s) for proteasomal degradation. These dual-specific bifunctional polypeptides may be used for the treatment of a disease in a subject, where the first and/or second component binds a biological target(s) whose abnormal function and/or expression is associated with the disease.

### CROSS-REFERENCE TO RELATED APPLICATIONS

This application claims priority to U.S. Ser. No. 63/379,509, filed Oct. 14, 2022, which is herein incorporated by reference in its entirety, including the accompanying sequence listing.

### REFERENCE TO SEQUENCE LISTING

The present application is being filed along with a Sequence Listing in electronic format. The Sequence Listing is provided as a file entitled 76BIO003WO_Sequence_Listing.xml, which was created on Oct. 12, 2023, and last modified on Oct. 13, 2023, which is 3,766,497 bytes in size. The information in the electronic Sequence Listing is hereby incorporated by reference in its entirety.

### FIELD

Aspects of the present disclosure relate generally to dual-specific bifunctional polypeptides that promote ubiquitin-mediated proteasomal degradation of desired biological targets, for example, for the treatment of a disease associated with the biological targets.

### BACKGROUND

Conventional therapeutics, such as small molecule and antibody inhibitors, operate by blocking or otherwise modulating the function of a therapeutic target (e.g., blocking an enzymatic or transcription promoting function). While many therapeutics belonging to this category have shown to be effective, confounding effects may arise as the therapeutic target is still present within the cellular milieu.

Molecular glues and proteolysis targeting chimeras (PROTACs) are alternative classes of therapeutics that involve small molecule compounds that recruit E3 ubiquitin ligases to a therapeutic target, thereby inducing proteolysis of the therapeutic target through the endogenous proteasomal degradation machinery. The physical degradation of the target drives a therapeutic effect through the elimination of the dysfunctional and/or disease-associated target. However, design of these small molecule degraders involves significant screening and optimizations to identify chemical modalities that are compatible for binding to both the therapeutic target and E3 ubiquitin ligase. Identification of such small molecule degraders is particularly challenging for previously undrugged intracellular targets, especially proteins that lack enzymatic domains amenable to traditional small molecule screening and optimization.

Therefore, there is a need for compounds designed to utilize a proteasomal degradation tactic, and improved approaches for identifying and/or assessing the same.

### SUMMARY OF THE DISCLOSURE

Disclosed herein are dual-specific bifunctional polypeptides that promote proteasome-mediated degradation of at least one target protein. In some embodiments, the dual-specific bifunctional polypeptide comprises a) a first targeting moiety (target binding domain) that is capable of binding to at least one target protein; b) a second targeting moiety that is capable of binding to at least one target protein; and c) a ubiquitin-proteasome system recruiting domain (URD). Optionally, in some embodiments the polypeptides further comprise d) a first linker peptide; wherein the first linker peptide is positioned between the first targeting moiety and the URD; and/or optionally e) a second linker peptide, wherein the second linker peptide is positioned between the second targeting moiety and the URD. In some embodiments, proximity of the dual-specific bifunctional polypeptide to either or both of the target proteins through binding of the targeting moiety induces ubiquitination of one or both of the target protein(s) via the URD, thereby promoting proteosome-mediated degradation of the target protein(s). In some embodiments, dual-specific bifunctional polypeptide is in a “contralateral” configuration in which the first and second target binding domains are on either side of the URD, i.e., one is on the C-terminal side of the URD, and one is on the N-terminal side of the URD. In some embodiments, dual-specific bifunctional polypeptide is in a “tandem” configuration in which the first and second target binding domains are both on the same side of the URD, i.e., both are on the C-terminal side of the URD, or both are on the N-terminal side of the URD. Optionally, in some embodiments a linker peptide (a first or a second linker peptide) is positioned between the two target binding domains, and/or between a targeting moiety and the URD. In some embodiments, the dual-specific bifunctional polypeptide comprises one or more localization sequences to direct the polypeptide to a preferred subcellular compartment (e.g., nucleus).

Also disclosed herein are polynucleotides encoding for any of the dual-specific bifunctional polypeptides provided herein.

Also disclosed herein are pharmaceutical compositions comprising any of the dual-specific bifunctional polypeptides provided herein and one or more pharmaceutically acceptable excipients, carriers, or diluents.

Also disclosed herein are methods of treating a subject. In some embodiments, the methods comprise administering any of the dual-specific bifunctional polypeptides, polynucleotides, or pharmaceutical compositions provided herein to a subject in need thereof. For example, the methods may be for the treatment of a cancer or a neurodegenerative disease.

Also disclosed herein are methods of reducing the amount of a target protein in a cell. In some embodiments, the methods comprise contacting the cell with any of the dual-specific bifunctional polypeptides, polynucleotides, or pharmaceutical compositions provided herein. In some embodiments, the cell is in a subject, and the dual-specific bifunctional polypeptide, polynucleotide, or pharmaceutical composition is administered to the subject. In some embodiments, the cell is contacted ex vivo, and after contacting the cell with the dual-specific bifunctional polypeptide, polynucleotide, or pharmaceutical composition, the cell is administered to a subject, optionally wherein the subject is also the source of the cell (e.g., in an adoptive cell therapy).

Some embodiments provided herein are described by way of the following provided numbered embodiments:

1. A dual-specific bifunctional polypeptide that promotes proteasome-mediated degradation of at least one target protein, comprising:
a) a first targeting moiety that is capable of binding to at least one target protein, optionally an intracellular target protein,
b) a second targeting moiety that is capable of binding to at least one target protein, optionally an intracellular target protein,
c) a ubiquitin-proteasome system recruiting domain (URD),
d) optionally a first linker peptide, wherein the first linker peptide is positioned between the first targeting moiety and the URD or between the second targeting moiety and the URD,
e) optionally a second linker peptide, wherein the second linker peptide is positioned between the second targeting moiety and the URD or between the first targeting moiety and the second targeting moiety, and
f) optionally one or more localization peptide sequences,
wherein proximity of the dual-specific bifunctional polypeptide to at least one target protein through binding of the first targeting moiety and/or the second targeting moiety induces ubiquitination of the at least one target protein via the URD, thereby promoting proteasome-mediated degradation of the at least one target protein.

2. The dual-specific bifunctional polypeptide of embodiment 1, wherein the first targeting moiety binds to and/or is designed to bind a first target protein and the second targeting moiety binds to and/or is designed to bind a second target protein.

3. The dual-specific bifunctional polypeptide of embodiment 1, wherein the first targeting moiety and the second targeting moiety bind to and/or are designed to bind to the same target protein.

4. The dual-specific bifunctional polypeptide of embodiment 3, wherein the first targeting moiety binds to and/or is capable of binding to a first binding site of the target protein and the second targeting moiety binds to and/or is capable of binding to a second binding site of the target protein.

5. The dual-specific bifunctional polypeptide of embodiment 3, wherein the first targeting moiety and second targeting moiety bind to and/or are capable of binding to the same binding site of the target protein, wherein the dual-specific bifunctional polypeptide engages and/or is capable of engaging at least two molecules of the at least one target protein.

6. The dual-specific bifunctional polypeptide of any one of embodiments 1-5, wherein the first targeting moiety and/or the second targeting moiety are less than or equal to 260, 259, 258, 257, 256, 255, 254, 253, 252, 251, 250, 245, 240, 235, 230, 225, 220, 215, 210, 205, 200, 195, 190, 185, 180, 175, 170, 165, 160, 155, 150, 145, 140, 135, 130, 129, 128, 127, 126, 125, 124, 123, 122, 121, 120, 119, 118, 117, 116, 115, 114, 113, 112, 110, 109, 108, 107, 106, 105, 104, 103, 102, 101, 100, 99, 98, 97, 96, 95, 94, 93, 92, 91, 90, 89, 88, 87, 86, 85, 84, 83, 82, 81, 80, 75, 70, 65, 60, 55, 54, 53, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 amino acids long, or a range defined by any two of the preceding values, optionally 10-260, 10-200, 10-130, 25-260, 25-200, 25-130, 35-260, 35-200, 35-130, 50-260, 50-200, or 50-130 amino acids long.

7. The dual-specific bifunctional polypeptide of any one of embodiments 1-6, wherein the first targeting moiety and/or the second targeting moiety comprises or consists of an endogenous binding partner of the at least one target protein, antibody, Fab, F(ab′)₂, Fab′, scFv, single domain antibody (sdAb), V_{H} domain, V_{L} domain, V_{H}H, V_{NAR}, diabody, intrabody, DARPin, monobody, affibody, avimer, or any fragment or derivative thereof.

8 The dual-specific bifunctional polypeptide of any one of embodiments 1-7, wherein the first targeting moiety and/or the second targeting moiety comprises or consists of an endogenous protein binding partner of the target protein, a fragment and/or a derivative thereof.

9. The dual-specific bifunctional polypeptide of embodiment 8, wherein the first targeting moiety and/or the second targeting moiety is, or is less than, 100, 99, 98, 97, 96, 95, 94, 93, 92, 91, 90, 89, 88, 87, 86, 85, 84, 83, 82, 81, 80, 75, 70, 65, 60, 55, 54, 53, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 amino acids long, or a range defined by any two of the preceding values, optionally 10-100, 10-25, 14-100, 14-25, 30-100, 30-50, 30-45, or 80-100 amino acids long.

10. The dual-specific bifunctional polypeptide of any one of embodiments 1-9, wherein the first targeting moiety and/or the second targeting moiety comprises or consists of an sdAb, optionally comprising or consisting of either a V_{H} domain or a V_{L} domain of an IgG antibody, or a V_{H}H, V_{H}, or V_{NAR} domain of a heavy chain antibody (HcAb), optionally wherein the sdAb comprises or consists of a V_{H}H or V_{L} domain.

11. The dual-specific bifunctional polypeptide of embodiment 10, wherein the first targeting moiety and/or the second targeting moiety is, or is less than, 170, 165, 160, 155, 150, 145, 140, 135, 130, 129, 128, 127, 126, 125, 124, 123, 122, 121, 120, 119, 118, 117, 116, 115, 114, 113, 112, 110, 109, 108, 107, 106, 105, 104, 103, 102, 101, 100, 99, 98, 97, 96, 95, 94, 93, 92, 91, or 90, amino acids long, or a range defined by any two of the preceding values, optionally 90-150, 90-130, 110-150, 110-130, 110-125, or 114-124 amino acids long.

12. The dual-specific bifunctional polypeptide of embodiment 1-11, wherein the first targeting moiety and/or the second targeting moiety comprises or consists of an scFv, optionally wherein the targeting moiety is, or is less than, 260, 259, 258, 257, 256, 255, 254, 253, 252, 251, 250, 245, 240, 235, 230, 225, 220, 215, 210, 205, 200, 195, or 190 amino acids long, or a range defined by any two of the preceding values, optionally 190-260, 200-260, or 230-260 amino acids long.

13. The dual-specific bifunctional polypeptide of any of embodiments 1-12, wherein the at least one target protein is selected from MYC, optionally c-MYC, beta catenin 1 (CTNNB1) and proliferating cell nuclear antigen (PCNA).

14. The dual-specific bifunctional polypeptide of any one of embodiments 1-13, wherein the at least one target protein is a wild-type and/or a mutant form of the target protein, optionally wherein the targeting moiety preferentially or selectively binds the mutant form of the target protein relative to the wild-type form of the target protein.

15. The dual-specific bifunctional polypeptide of embodiment 14, wherein the mutant form of the target protein is CTNNB1 (SEQ ID NO: 3) comprising one or more mutations selected from: D32Y; D32N; D32V; D32G; S33Y; S33L; S33F; S33C; S33P; G34V; G34E; I35S; H36P; S37C; S37P; S37P and D207G; T41A; T41I; T42_K49del; S45F and Y670X wherein X indicates termination; S45C; S45F; S45P; S45Y; and S45del.

16. The dual-specific bifunctional polypeptide of any one of embodiments 1-15, wherein the first targeting moiety and/or the second targeting moiety bind, or bind at least in part, to at least a portion of:
a) the DNA binding domain of MYC;
b) the armadillo domain of CTNNB1; or
c) the p21 binding domain of PCNA.

17. The dual-specific bifunctional polypeptide of any one of embodiments 1-16, wherein the first targeting moiety and/or second targeting moiety binds, or binds at least in part, to at least a portion of the target protein selected from the portion consisting of:
a) amino acids 54-406 of MYC (SEQ ID NO: 1),
b) amino acids 354-406 of MYC (SEQ ID NO: 1),
c) amino acids 408-437 of MYC (SEQ ID NO: 1),
d) amino acids 410-419 of MYC (SEQ ID NO: 1),
e) amino acids 150-663 of CTNNB1 (SEQ ID NO: 3),
f) amino acids 15-29 of CTNNB1 (SEQ ID NO: 3),
g) amino acids 249-265, 292-306, 379-390, 415-429, 462-470, or 505-519, or any combination thereof, of CTNNB1 (SEQ ID NO: 3), and
h) amino acids 38-48, 123-129, or 251-257 or any combination thereof, of PCNA (SEQ ID NO: 2151).

18. The dual-specific bifunctional polypeptide of any one of the preceding embodiments, wherein the first targeting moiety and/or the second targeting moiety are selected from Omomyc, TCF4, TCF4.1, TCF4.2, TCF4.3, TCF4.4, TCF4.5, TCF4.6, TCF4.7, TCF4.8, TCF4.9, TCF4.10, TCF4.11, TCF4.12, TCF4.13, TCF4.14, TCF4.15, TCF4.16, con1, con1.1, con1.2, p21, p21.1, p21.2, p21.3, p21.4, p21.5, p21.6, p21.7, p21.8, p21.9, mycV.0300, mycV.1300, mycV.1500, mycV.3700, mycV.6600, mycV.1516, mycV.1515, mycV.1514, mycV.1513, mycV.1512, mycV.1511, mycV.1510, mycV.1509, mycV.1508, mycV.1507, mycV.1506, mycV.1505, mycV.1504, mycV.1503, mycV.1502, mycV.1501, or a fragment thereof that is capable of binding to the target protein, optionally wherein the targeting moiety does not comprise full length or unmodified con1 or p21.

19. The dual-specific bifunctional polypeptide of any one of the preceding embodiments, wherein the first and/or the second targeting moiety comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NO:6, 11, 12, 1918, 1926, 1934, 1942, 1950, 1958, 1966, 1974, 1982, 1990, 1998, 2006, 2014, 2022, 2030, 2038, 2046, 2054, 2062, 2070, 2078, 2086, and 2087-2112.

20. The dual-specific bifunctional polypeptide of any one of the preceding embodiments, wherein the dual-specific bifunctional polypeptide comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NOs: 2172-2207 and 2209-2351, or optionally 2172-2207 and 2209-2286, optionally wherein the sequence lacks the HA tag sequence (SEQ ID NO: 50).

21. The dual-specific bifunctional polypeptide of any one of embodiments 1-20, wherein the dual-specific bifunctional polypeptide comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of the SEQ ID NOs in Tables 7, 10 and 13 listed as having a degradation Grade of A for at least one of the target proteins, degradation Grade of B for at least one of the target proteins, degradation Grade of C for at least one of the target proteins, and/or degradation Grade of D for at least one of the target proteins, optionally excluding any one of the SEQ ID NOs listed as having a degradation Grade of D for at least one of the target proteins, optionally wherein the sequence lacks the HA tag sequence (SEQ ID NO: 50).

22. The dual-specific bifunctional polypeptide of any one of embodiments 1-21, wherein the dual-specific bifunctional polypeptide comprises or consists of an amino acid sequence of any one of the SEQ ID NOs in Tables 7, 10 and 13 listed as having degradation Grade of A for at least one of the target proteins, and/or degradation Grade of B for at least one of the target proteins, optionally wherein the sequence lacks the HA tag sequence (SEQ ID NO: 50).

23. The dual-specific bifunctional polypeptide of any one of embodiments 1-22, wherein the dual-specific bifunctional polypeptide comprises or consists of an amino acid sequence of any one of the SEQ ID NOs in Tables 7, 10 and 13 listed as having a degradation Combined Grade of A/A or A, optionally wherein the sequence lacks the HA tag sequence (SEQ ID NO: 50).

24. The dual-specific bifunctional polypeptide of any one of embodiments 1-22, wherein the dual-specific bifunctional polypeptide comprises or consists of an amino acid sequence of any one of the SEQ ID NOs in Tables 7, 10 and 13 listed as having a degradation Combined Grade of A/B or B/A, optionally wherein the sequence lacks the HA tag sequence (SEQ ID NO: 50).

25. The dual-specific bifunctional polypeptide of any one of embodiments 1-22, wherein the dual-specific bifunctional polypeptide comprises or consists of an amino acid sequence of any one of the SEQ ID NOs in Tables 7, 10 and 13 listed as having a degradation Combined Grade of B/B or B, optionally wherein the sequence lacks the HA tag sequence (SEQ ID NO: 50).

26. The dual-specific bifunctional polypeptide of any one of embodiments 1-25, wherein the dual-specific bifunctional polypeptide does not comprise or consist of an amino acid sequence of any one of the SEQ ID NOs in Tables 7, 10 and 13 listed as having a degradation Grade of C and/or D for at least one of the target proteins, optionally wherein the sequence lacks the HA tag sequence (SEQ ID NO: 50).

27. The dual-specific bifunctional polypeptide of any one of embodiments 1-26, wherein the dual-specific bifunctional polypeptide comprises or consists of an amino acid sequence of any one of the SEQ ID NOs in Tables 8, 11, and 14, and FIGS. 10-19, optionally wherein the western blot shows degradation of one and/or both of the target proteins.

28. The dual-specific bifunctional polypeptide of any one of embodiments 1-27, wherein the URD is derived from at least one E3 ubiquitin ligase.

29. The dual-specific bifunctional polypeptide of any one of embodiments 1-28, wherein the URD is derived from a RING family E3 ubiquitin ligase, an H-box family E3 ubiquitin ligase or a viral analogue of an E3 ubiquitin ligase.

30. The dual-specific bifunctional polypeptide of any one of embodiments 1-29, wherein the URD is selected from:
a) the URD derived from a protein which localizes to the cytoplasm, optionally selected from FBXW7 beta and Keap1;
b) the URD derived from a protein which localizes to the nucleus, optionally selected from FBXW7 alpha, RNF165, and SPOP, optionally SPOP.2; and
c) the URD derived from a protein which localizes to the cytoplasm and the nucleus, optionally selected from beta-TRCP, CHIP, RNF114, RNF125, RNF138, RNF166, NHLRC1, CBL-b (Y363E), TRIM21, E6AP, DCAF1 VIF, RNF11, RNF111, RNF115, RNF12, RNF128, RNF149, RNF152, RNF165, RNF182, RNF20, RNF25, RNF4, RNF6, TRIM32, ZNRF1, ZNRF4;
d) the URD derived from an E3 ligase selected from the group consisting of NHLRC1, DCAF1, RNF125, RNF165, RNF4, and SPOP, optionally NHLRC1, RNF125, RNF165, and RNF4 or
e) a truncation or fragment of the URD that retains ubiquitin-proteasome recruiting activity, optionally NHLRC1.1, DCAF1.1, RNF125.2, RNF125.3, RNF125.4, RNF125.5, RNF125.6, RNF165.1, RNF4.1, SPOP.2, and SPOP.3;
optionally wherein the protein is an E3 ubiquitin ligase.

31. The dual-specific bifunctional polypeptide of any one of embodiments 1-27, wherein the URD is derived from a RING family E3 ubiquitin ligase, a cullin family E3 ubiquitin ligase, a homologous to E6AP carboxyl terminus (HECT) family E3 ubiquitin ligase, and a viral homolog of an E3 ubiquitin ligase.

32. The dual-specific bifunctional polypeptide of any one of embodiments 1-27, wherein the URD is derived from an E3 ubiquitin ligase selected from the group consisting of CHIP, DCAF1, E6AP, FBXW7-alpha, FBXW7-beta, Keap1, NHLRC1, RNF4, RNF6, RNF11, RNF12, RNF20, RNF25, RNF111, RNF114, RNF115, RNF125, RNF128, RNF138, RNF149, RNF152, RNF165, RNF166, RNF182, SPOP, beta-TRCP, TRIM21, TRIM32, VIF, ZNRF1, ZNRF4, and CBL-b (Y363E), optionally wherein the portion is selected from the group consisting of CBLb.1, CHIP.1, DCAF1.1, E6AP.1, FBXW7a.1, FBXW7a.2, FBXW7a.3, FBXW7a.4, FBXW7a.5, FBXW7a.6, FBXW7a.7, FBXW7a.8, FBXW7a.9, FBXW7b, FBXW7b.2, FBXW7b.3, FBXW76.4, Keap1.1, NHLRC1.1, NHLRC1.2, NHLRC1.3, NHLRC1.4, NHLRC1.5, RNF4.1, RNF6.1, RNF6.2, RNF11.1, RNF12.1, RNF12.2, RNF20.1, RNF25.1, RNF111.1, RNF114.1, RNF115.1, RNF125.1, RNF125.2, RNF125.3, RNF125.4, RNF125.5, RNF125.6, RNF125.7, RNF128.1, RNF138.1, RNF149.1, RNF152.1, RNF165.1, RNF166.1, RNF182.1, RNF182.2, SPOP.1, SPOP.2, SPOP.3, bTRCP.1, TRIM21.1, TRIM21.2, TRIM21.3, TRIM32.1, VIF.1, ZNRF1.1, and ZNRF4.1.

33. The dual-specific bifunctional polypeptide of any one of embodiments 1-27, wherein the URD is derived from a monomeric RING family E3 ligase.

34. The dual-specific bifunctional polypeptide of any one of embodiments 1-27, wherein the URD is derived from an E3 ligase selected from the group consisting of NHLRC1, RNF11, RNF111, RNF114, RNF115, RNF12, RNF125, RNF128, RNF138, RNF149, RNF152, RNF165, RNF166, RNF182, RNF20, RNF25, RNF4, RNF6, ZNRF1, ZNRF4, and CBL-b (Y363E), or optionally selected from the group consisting of RNF125, NHLRC1, RNF4, RNF6, RNF12, RNF138 and ZNRF1.

35. The dual-specific bifunctional polypeptide of any one of embodiments 1-27, wherein the URD is selected from the group consisting of NHLRC1.1, NHLRC1.2, NHLRC1.3, NHLRC1.4, NHLRC1.5, RNF11.1, RNF111.1, RNF114.1, RNF115.1, RNF12.1, RNF12.2, RNF125.1, RNF125.2, RNF125.3, RNF125.4, RNF125.5, RNF125.6, RNF125.7, RNF128.1, RNF138.1, RNF149.1, RNF152.1, RNF165.1, RNF166.1, RNF182.1, RNF182.2, RNF20.1, RNF25.1, RNF4.1, RNF6.1, RNF6.2, ZNRF1.1, ZNRF4.1, and CBLb.1, optionally selected from the group consisting of NHLRC1.1, NHLRC1.2, NHLRC1.3, NHLRC1.4, NHLRC1.5, RNF125.1, RNF125.2, RNF125.3, RNF125.4, RNF125.5, RNF125.6, RNF125.7, RNF4.1, RNF6.1, RNF6.2, RNF12.1, RNF12.2, RNF138.1, RNF165.1, and ZNRF1.1.

36. The dual-specific bifunctional polypeptide of any one of embodiments 1-27, wherein the URD is derived from a cullin family E3 ligase.

37. The dual-specific bifunctional polypeptide of any one of embodiments 1-27, wherein the URD is derived from an E3 ligase selected from the group consisting of DCAF1, beta-TRCP, FBXW7-alpha, FBXW7-beta, Keap1, and SPOP, optionally selected from the group consisting of beta-TRCP, FBXW7-alpha, and FBXW7-beta.

38. The dual-specific bifunctional polypeptide of any one of embodiments 1-27, wherein the URD is selected from the group consisting of DCAF1.1, bTRCP.1, FBXW7a.1, FBXW7a.2, FBXW7a.3, FBXW7a.4, FBXW7a.5, FBXW7a.6, FBXW7a.7, FBXW7a.8, FBXW7a.9, FBXW7b, FBXW7b.2, FBXW7b.3, FBXW7b.4, Keap1.1, SPOP.1, SPOP.2, and SPOP.3.

39. The dual-specific bifunctional polypeptide of any one of embodiments 1-27, wherein the URD is derived from an E3 ligase NHLRC1, DCAF1, RNF125, RNF165, RNF4, and SPOP, optionally NHLRC1, RNF125, RNF165, RNF4, and SPOP, optionally NHLRC1, RNF125, RNF165, and RNF4, and/or wherein the URD is selected from the group consisting of NHLRC1.1, DCAF1.1, RNF125.2, RNF125.3, RNF125.4, RNF125.5, RNF125.6, RNF165.1, RNF4.1, SPOP.2, and SPOP.3, optionally NHLRC1.1, RNF125.2, RNF125.3, RNF125.4, RNF125.5, RNF125.6, RNF165.1, RNF4.1, SPOP.2, and SPOP.3, optionally NHLRC1.1, RNF125.2, RNF165.1, and RNF4.1.

40. The dual-specific bifunctional polypeptide of any one of embodiments 1-39, wherein the number of amino acids in the URD is, is about, or is less than, 80, 75, 70, 65, 60, 65, 50, 45, 40, 35, 30, 25, 20, 15, 10, 5, 4, 3, 2, or 1%, of the number of amino acids in the wild-type full-length protein from which it is derived, or a range defined by any two of the preceding values, optionally 15-80, 15-50, 25-80, 25-50, 30-60, 1-80, 2-80, 1-70, 2-70, 2-65%, optionally 15-50%, optionally 2-61%.

41. The dual-specific bifunctional polypeptide of any one of embodiments 1-40, wherein the URD is less than or equal to 370, 369, 368, 367, 366, 365, 364, 363, 362, 361, 360, 300, 250, 200, 175, 170, 165, 160, 155, 150, 145, 140, 135, 130, 129, 128, 127, 126, 125, 124, 123, 120, 115, 110, 105, 100, 95, 90, 85, 80, 75, 70, 65, 60, 55, 50, 47, 46, 45, 44, 43, 42, 41, 40, 39, 38, 37, 36, 35, 34, 33, 32, 31, 30, 25, or 20 amino acids long, or a range defined by any two of the preceding values, optionally 20-370, 20-150, 20-100, 20-80, 30-150, 30-100, 50-150, or 50-100, optionally 20-100 amino acids long, optionally 40-130 or 44-126.

42. The dual-specific bifunctional polypeptide of any one of embodiments 1-41, wherein the URD comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NO: 29-43, 1836-1895, optionally any one of SEQ ID NO: 1837, 1855, 1856, 1858, 1870-1874, 1880, and 1886, optionally any one of SEQ ID NO: 1855, 1856, 1858, 1870-1874, 1880, and 1886, optionally any one of SEQ ID NO: 1858, 1870, 1880, and 1886, or a truncation or fragment thereof that retains ubiquitin-proteasome recruiting activity.

43. The dual-specific bifunctional polypeptide of any one of the preceding embodiments, further comprising one or more linker peptides, wherein the linker peptide is positioned between one or more of: the first targeting moiety and the URD; the second targeting moiety and the URD; the first targeting moiety and the second targeting moiety; a localization peptide and the first targeting moiety; a localization peptide and the second targeting moiety; a localization peptide and the URD; and/or a localization peptide and an epitope tag, optionally an HA tag.

44. The dual-specific bifunctional polypeptide of any one of the preceding embodiments, comprising a first linker peptide positioned between the first targeting moiety and the URD or between the second targeting moiety and the URD, and a second linker peptide positioned between the second targeting moiety and the URD or between the first targeting moiety and the second targeting moiety, optionally wherein the first linker peptide is positioned between the first targeting moiety and the URD and the second linker peptide is positioned between the second targeting moiety and the URD.

45. The dual-specific bifunctional polypeptide of any one of embodiments 1-44, wherein the one or more linker peptides is independently 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids in length, or a range defined by any two of the preceding values, optionally 0-30, 1-30, 2-30, 0-20, 1-20, 2-20, 0-15, 0-12, 0-10, 1-15, 1-12, 1-10, 2-15, 2-12, 2-10, 3-15, 3-12, or 3-10 amino acids in length, optionally 2~30 amino acids in length, 46. The dual-specific bifunctional polypeptide of any one of embodiments 1-45, wherein the at least one or more linker peptides comprise or consist of glycine and serine.

47. The dual-specific bifunctional polypeptide of any one of embodiments 1-46, wherein the one or more linker peptides comprises or consists of one or more alpha helixes, optionally flanked by one or more glycine and/or a serine residues.

48. The dual-specific bifunctional polypeptide of any one of embodiments 1-47, wherein the one or more linker peptides independently comprise or consist of the amino acid sequence of any one of SEQ ID NO: 44-49 and 2113-2151, or 44-49.

49. The dual-specific bifunctional polypeptide of any one of the preceding embodiments, further comprising one or more localization peptide sequences.

50. The dual-specific bifunctional polypeptide of embodiment 49, wherein the one or more localization peptide sequences direct the dual-specific bifunctional polypeptide to a subcellular compartment or compartments, optionally wherein the subcellular compartment is the nucleus and/or the cytoplasm.

51. The dual-specific bifunctional polypeptide of embodiment 49 or 50, wherein the one or more localization peptide sequences direct the dual-specific bifunctional polypeptide to a desired subcellular compartment or compartments, optionally wherein the one or more localization peptide sequences are selected or designed to direct the dual-specific bifunctional polypeptide to a desired subcellular compartment or compartments.

52. The dual-specific bifunctional polypeptide of any one of embodiments 49-51, wherein the one or more localization peptide sequences comprise or consist of a nuclear localization signal (NLS) peptide and/or a nuclear export signal (NES) peptide, optionally wherein the localization peptide comprises or consists of an amino acid sequence of any one of SEQ ID NO: 51 and 1896-1909.

53. The dual-specific bifunctional polypeptide of any one of embodiments 49-52, wherein the one or more localization peptide sequences comprise or consist of an endogenous localization peptide sequence of the first and/or second targeting moiety and/or the URD.

54. The dual-specific bifunctional polypeptide of embodiment 53, wherein the one or more localization peptide sequences comprise or consist of an endogenous localization peptide sequence of the URD.

55. The dual-specific bifunctional polypeptide of embodiment 54, wherein the endogenous localization peptide sequence is from a URD selected from:
a URD derived from a protein which localizes to the cytoplasm, optionally selected from FBXW7 beta and Keap1;
a URD derived from a protein which localizes to the nucleus, optionally selected from FBXW7 alpha, RNF165, and SPOP; and
a URD derived from a protein which localizes to the cytoplasm and the nucleus, optionally selected from beta-TRCP, CHIP, RNF114, RNF125, RNF138, RNF166, NHLRC1, CBL-b (Y363E), TRIM21, E6AP, DCAF1 VIF, RNF11, RNF111, RNF115, RNF12, RNF128, RNF149, RNF152, RNF165, RNF182, RNF20, RNF25, RNF4, RNF6, TRIM32, ZNRF1, ZNRF4.

56. The dual-specific bifunctional polypeptide of any one of embodiment s 49-55, wherein the one or more localization peptide sequences comprise or consist of an endogenous localization peptide sequence of the first and/or second targeting moiety and/or the URD that is modified, wherein the modification alters the subcellular compartment or compartments to which the dual-specific bifunctional polypeptide is directed as compared to the native endogenous localization peptide sequence.

57. The dual-specific bifunctional polypeptide of embodiment 56, wherein the modification comprises or consists of a substitution, truncation or deletion of an endogenous localization peptide sequence.

58. The dual-specific bifunctional polypeptide of embodiment 56 or 57, wherein:
a. the native endogenous localization peptide sequence directs the polypeptide to the nucleus, and the modified endogenous localization peptide sequence directs the polypeptide to the cytoplasm;
b. the native endogenous localization peptide sequence directs the polypeptide to the cytoplasm, and the modified endogenous localization peptide sequence directs the polypeptide to the nucleus;
c. the native endogenous localization peptide sequence directs the polypeptide to the nucleus and the cytoplasm, and the modified endogenous localization peptide sequence directs the polypeptide to only the cytoplasm;
d. the native endogenous localization peptide sequence directs the polypeptide to the nucleus and the cytoplasm, and the modified endogenous localization peptide sequence directs the polypeptide to only the nucleus;
e. the native endogenous localization peptide sequence directs the polypeptide to the nucleus, and the modified endogenous localization peptide sequence directs the polypeptide to the nucleus and the cytoplasm; or
f. the native endogenous localization peptide sequence directs the polypeptide to the cytoplasm, and the modified endogenous localization peptide sequence directs the polypeptide to the nucleus and the cytoplasm.

59. The dual-specific bifunctional polypeptide of embodiment 56 or 57, wherein the one or more localization peptide sequences comprises or consists of an endogenous localization peptide sequence of the URD that is modified, wherein the native endogenous localization peptide sequence directs the polypeptide to the nucleus, and the modified endogenous localization peptide sequence directs the polypeptide to the cytoplasm.

60. The dual-specific bifunctional polypeptide of any one of embodiments 56-59, wherein the modified endogenous localization peptide sequence comprises or consists of a modified SPOP URD endogenous localization peptide sequence, optionally wherein the URD is SPOP.3.

61. The dual-specific bifunctional polypeptide of any one of embodiments 49-60, wherein the one or more localization peptide sequences comprise or consist of an exogenous localization peptide sequence.

62. The dual-specific bifunctional polypeptide of any one of embodiments 49-61, wherein the one or more localization peptide sequences comprise or consist of an NLS peptide that is, or is derived from the NLS of MYC, simian virus 40 (SV40), SPOP, nucleoplasmin, 53BP1, or Hrp1.

63. The dual-specific bifunctional polypeptide of any one of embodiments 49-62, wherein the one or more localization peptide sequences comprise or consist of an NLS or NES comprising or consisting of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NO: 51 or 1896-1917.

64. The dual-specific bifunctional polypeptide of any one of embodiments 49-63, wherein the dual-specific bifunctional polypeptide comprises or consists of two localization peptide sequences, optionally wherein the dual-specific bifunctional polypeptide comprises or consists of 3, 4, or more localization peptide sequences, optionally wherein the wherein the dual-specific bifunctional polypeptide comprises or consists of 2, 3, 4 or more copies of the same localization sequence in series.

65. The dual-specific bifunctional polypeptide of any one of embodiments 49-64, comprising one or more localization peptide sequences located at a location selected from the group consisting of the N-terminus of the dual-specific bifunctional polypeptide; the C-terminus of the dual-specific bifunctional polypeptide; internally within the dual-specific bifunctional polypeptide; as part of the URD, optionally an endogenous portion of the URD; as a part of the first and/or second targeting moiety, optionally an endogenous portion of the first and/or second targeting moiety; and a combination of any of the foregoing.

66. The dual-specific bifunctional polypeptide of any one of embodiments 49-65, wherein a first localization peptide sequence is located at the N-terminus of the dual-specific bifunctional polypeptide and a second localization peptide sequence is located at the C-terminus of the dual-specific bifunctional polypeptide.

67. The dual-specific bifunctional polypeptide of any one of embodiments 1-66, further comprising at least one NLS peptide, wherein the at least one NLS peptide is located at the N-terminus of the dual-specific bifunctional polypeptide, the C-terminus of the dual-specific bifunctional polypeptide, is part of the URD, optionally an endogenous NLS of the URD, or is part of the first and/or second targeting moiety, optionally an endogenous NLS of the first and/or second targeting moiety.

68. The dual-specific bifunctional polypeptide of embodiment 1-67, wherein a first NLS peptide is located at the N-terminus of the dual-specific bifunctional polypeptide and a second NLS peptide is located at the C-terminus of the dual-specific bifunctional polypeptide.

69. The dual-specific bifunctional polypeptide of any one of embodiments 1-68, wherein the orientation of the first targeting moiety relative to the URD is the same orientation as compared to the naturally occurring substrate recognition domain of the protein from which the URD is derived.

70. The dual-specific bifunctional polypeptide of any one of embodiments 1-69, wherein the orientation of the first targeting moiety relative to the URD is the opposite orientation as compared to the naturally occurring substrate recognition domain of the protein from which the URD is derived.

71. The dual-specific bifunctional polypeptide of any one of embodiments 1-70, wherein the orientation of the second targeting moiety relative to the URD is the same orientation as compared to the naturally occurring substrate recognition domain of the protein from which the URD is derived.

72. The dual-specific bifunctional polypeptide of any one of embodiments 1-71, wherein the orientation of the second targeting moiety relative to the URD is the opposite orientation as compared to the naturally occurring substrate recognition domain of the protein from which the URD is derived.

73. The dual-specific bifunctional polypeptide of any one of embodiments 1-72, wherein the first targeting moiety is N-terminal relative to the URD.

74. The dual-specific bifunctional polypeptide of any one of embodiments 1-73, wherein the second targeting moiety is C-terminal relative to the URD.

75. The dual-specific bifunctional polypeptide of any one of embodiments 1-72, wherein the first and the second targeting moieties are N-terminal relative to the URD.

76. The dual-specific bifunctional polypeptide of any one of embodiments 1-72, wherein the first and the second targeting moieties are C-terminal relative to the URD.

77. The dual-specific bifunctional polypeptide of any one of embodiments 1-76, further comprising an epitope tag, optionally a hemagglutinin (HA) tag, optionally wherein the HA tag comprises or consists of the amino acid sequence of SEQ ID NO: 50, wherein the epitope tag can be used to visualize the dual-specific bifunctional polypeptide. 78. The dual-specific bifunctional polypeptide of any one of embodiments 1-76, wherein the dual-specific bifunctional polypeptide does not comprise an epitope tag, optionally a hemagglutinin (HA) tag, optionally wherein the dual-specific bifunctional polypeptide does not comprise an HA tag comprising or consisting of the amino acid sequence of SEQ ID NO: 50.

79. The dual-specific bifunctional polypeptide of any one of the preceding embodiments, wherein the dual-specific bifunctional polypeptide is less than or equal to 60, 70, 80, 90, 100, 110, 120, 130, 140, 150, 160, 170, 180, 190, 200, 250, 300, 350, 400, 450, 500, 550, 600, 650, 700, 750, 800, 850, 900, 950. 1000, 1050, 1100, or 1150, amino acids long, or a range defined by any two of the preceding values, optionally 120-400 or 130-370 amino acids long.

80. The dual-specific bifunctional polypeptide of any one of embodiments 1-79, wherein the dual-specific bifunctional polypeptide comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to the peptide of any one of SEQ ID NO: 2172-2207 and 2209-2351, or optionally 2172-2207 and 2209-2286.

81. The dual-specific bifunctional polypeptide of any one of embodiments 1-78, wherein the at least one target protein comprises MYC.

82. The dual-specific bifunctional polypeptide of embodiment 81, wherein the dual-specific bifunctional polypeptide comprises a localization peptide sequence which directs the dual-specific bifunctional polypeptide to the nucleus of a cell, optionally wherein the localization peptide sequence comprises or consists of the NLS sequence selected from the group of the NLS sequences of MYC, SPOP, Hrp1, SV40, and nucleoplasmin, optionally wherein the localization peptide sequence comprises or consists of a NLS sequence selected from the group of the NLS sequence of SPOP, optionally SPNLS.2, MYC, and Hrp1.

83. The dual-specific bifunctional polypeptide of embodiment 82, wherein the NLS comprises or consists of a sequence selected from any one of SEQ ID NOs: 51, 1896-1909, optionally wherein the NLS comprises or consists of a sequence selected from any one of SEQ ID NOs: 1904, 1896 and 1898.

84. The dual-specific bifunctional polypeptide of any one of embodiments 81-83, wherein the URD is the URD of NHLRC1, RNF4, RNF125, RNF165, or SPOP, or a truncation or fragment thereof that retains ubiquitin-proteasome recruiting activity, optionally NHLRC1.1, RNF4.1, RNF125.2, RNF165.1, or SPOP.2, or optionally NHLRC1.1, RNF4.1, RNF125.2, or RNF165.1, or optionally RNF165.1, RNF4.1, or RNF125.2.

85. The dual-specific bifunctional polypeptide of any one of embodiments 81-84, wherein the URD comprises or consists of a sequence selected from any one of SEQ ID NOs: 29-42 and 1836-1895, optionally wherein the URD comprises or consists of a sequence selected from any one of SEQ ID NOs: 1858, 1886, 1870, 1880, and 1855, or optionally SEQ ID NOs: 1858, 1886, 1870, and 1880, or optionally SEQ ID NOs: 1886, 1870, and 1880.

86. The dual-specific bifunctional polypeptide of any one of embodiments 81-85, wherein the first and/or second targeting moiety binds, or binds at least in part, to at least a portion of:
a.) the DNA binding domain of MYC;
b.) amino acids 54-406 of MYC (SEQ ID NO: 1);
c.) amino acids 354-406 of MYC (SEQ ID NO: 1);
d.) amino acids 408-437 of MYC (SEQ ID NO: 1); and
e.) amino acids 410-419 of MYC (SEQ ID NO: 1).

87. The dual-specific bifunctional polypeptide of any one of embodiments 81-86, wherein the first and/or second targeting moiety comprises or consists of an endogenous protein binding partner of the target protein, a fragment and/or a derivative thereof.

88. The dual-specific bifunctional polypeptide of any one of embodiments 81-87, wherein the first targeting moiety and/or the second targeting moiety is Omomyc, or a fragment thereof that is capable of binding to the target protein, optionally comprising or consisting of an amino acid sequence having at least 80%, 85%, 86%, 87%, 88%, 89%, 90%, 91%, 92%, 93%, 94%, 95%, 96%, 97%, 98%, 99%, or 100% sequence identity to SEQ ID NO: 2086.

89. The dual-specific bifunctional polypeptide of any one of embodiments 81-88, wherein the dual-specific bifunctional polypeptide comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NO: 2189-2201 and 2284-2286, optionally any one of SEQ ID NO: 2189-2193, optionally wherein the sequence lacks the HA tag sequence (SEQ ID NO: 50).

90. The dual-specific bifunctional polypeptide of any one of embodiments 81-86, wherein the first and/or second targeting moiety comprises or consists of a sdAb, optionally comprising or consisting of either a V_{H} domain or a V_{L} domain of an IgG antibody, or a V_{H}H, V_{H}, or V_{NAR} domain of a heavy chain antibody (HcAb), optionally wherein the sdAb comprises or consists of a V_{H}H domain.

91. The dual-specific bifunctional polypeptide of embodiment 90, wherein the first and/or second targeting moiety comprises or consists of a sdAb, optionally a V_{H}H domain, comprising a CDR1 selected from any one of SEQ ID NOs: 1920, 1928, 1944, and 1952, a CDR2 selected from any one of SEQ ID NOs: 1922, 1930, 1938, and 1946, and a CDR3 selected from any one of SEQ ID NOs: 1924, 1932, 1940, 1948, and 1956; optionally wherein the combination of CDR1, CDR2, and CDR3 is SEQ ID NOs: 1936, 1938 and 1940;
optionally wherein each CDR sequence independently comprises 1, 2, 3, 4, 5, 6, 7, or more substitutions and/or 1, 2, 3, 4, 5, 6, 7, or more deletions or additions, and/or has at least 90%, 91%, 92%, 93%, 94%, 95%, 96%, 97%, 98%, 99%, or 100% sequence identity to a sequence selected from any one of SEQ ID NOs: 1920, 1928, 1944, 1952, 1922, 1930, 1938, 1946, 1924, 1932, 1940, 1948, and 1956.

92. The dual-specific bifunctional polypeptide of embodiment 91, wherein the sdAb, optionally a V_{H}H domain, comprises a V_{H} FR1 selected from any one of SEQ ID NOs: 1919 and 1951, a V_{H} FR2 of SEQ ID NO: 1921, a V_{H} FR3 selected from any one of SEQ ID NOs: 1923, 1939, and 1955, and a V_{H} FR4 selected from any one of SEQ ID NOs: 1925 and 1933, optionally wherein the combination of FR1, FR2, FR3 and FR4 is SEQ ID NOs: 1935, 1937, 1939, and 1941;
optionally wherein each V_{H} framework region comprises 1, 2, 3, 4, 5, 6, 7, 8, 9, 10, 11, 12, 13, 14, 15, 16, 17 or more substitutions and/or 1, 2, 3, 4, 5, 6, 7, 8, 9, 10, 11, 12, 13, 14, 15, 16, 17 or more deletions or additions, and/or has at least 90%, 91%, 92%, 93%, 94%, 95%, 96%, 97%, 98%, 99%, or 100% sequence identity to a sequence selected from any one of SEQ ID NOs: 1919, 1951, 1921, 1923, 1939, 1955, 1925, and 1933.

93. The dual-specific bifunctional polypeptide of any one of embodiments 90-92, wherein the first and/or second targeting moiety comprises or consists of a sdAb, optionally a V_{H}H domain, sequence selected from any one of SEQ ID NOs: 1918, 1926, 1934, 1942, 1950, 1958, 1966, 1974, 1982, 1990, 1998, 2006, 2014, 2022, 2030, 2038, 2046, 2054, 2062, 2070, and 2078, optionally wherein each CDR sequence independently comprises 1, 2, 3, 4, 5, 6, 7, or more substitutions and/or 1, 2, 3, 4, 5, 6, 7, or more deletions or additions, and/or has at least 90%, 91%, 92%, 93%, 94%, 95%, 96%, 97%, 98%, 99%, or 100% sequence identity to a CDR sequence of any one of SEQ ID NOs: 1918, 1926, 1934, 1942, 1950, 1958, 1966, 1974, 1982, 1990, 1998, 2006, 2014, 2022, 2030, 2038, 2046, 2054, 2062, 2070, and 2078, and/or wherein the framework region comprises 1, 2, 3, 4, 5, 6, 7, 8, 9, 10, 11, 12, 13, 14, 15, 16, 17 or more substitutions and/or 1, 2, 3, 4, 5, 6, 7, 8, 9, 10, 11, 12, 13, 14, 15, 16, 17 or more deletions or additions, and/or has at least 90%, 91%, 92%, 93%, 94%, 95%, 96%, 97%, 98%, 99%, or 100% sequence identity to the framework region of any one of SEQ ID NOs: 1918, 1926, 1934, 1942, 1950, 1958, 1966, 1974, 1982, 1990, 1998, 2006, 2014, 2022, 2030, 2038, 2046, 2054, 2062, 2070, and 2078.

94. The dual-specific bifunctional polypeptide of any one of embodiments 90-93, wherein the bifunctional polypeptide comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NOs: 2176-2188, and 2282-2283, optionally wherein the sequence lacks the HA tag sequence (SEQ ID NO: 50).

95. The dual-specific bifunctional polypeptide of any one of embodiments 90-94, wherein the bifunctional polypeptide comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of the SEQ ID NOs in Table 10, optionally excluding any one of the SEQ ID NOs listed as having a degradation Grade of D for at least one of the target proteins, optionally wherein the sequence lacks the HA tag sequence (SEQ ID NO: 50).

96. The dual-specific bifunctional polypeptide of any one of embodiments 90-95, wherein the bifunctional polypeptide comprises or consists of an amino acid sequence of any one of the SEQ ID NOs in Table 10 listed as having a MYC degradation Grade of A, and/or MYC degradation Grade of B, optionally wherein the sequence lacks the HA tag sequence (SEQ ID NO: 50).

97. The dual-specific bifunctional polypeptide of any one of embodiments 90-95, wherein the dual-specific bifunctional polypeptide comprises or consists of an amino acid sequence of any one of the SEQ ID NOs in Table 10 listed as having a degradation Combined Grade of A/A or A, optionally wherein the sequence lacks the HA tag sequence (SEQ ID NO: 50).

98. The dual-specific bifunctional polypeptide of any one of embodiments 90-95, wherein the dual-specific bifunctional polypeptide comprises or consists of an amino acid sequence of any one of the SEQ ID NOs in Table 10 listed as having a degradation Combined Grade of A/B or B/A, optionally wherein the sequence lacks the HA tag sequence (SEQ ID NO: 50).

99. The dual-specific bifunctional polypeptide of any one of embodiments 90-95, wherein the dual-specific bifunctional polypeptide comprises or consists of an amino acid sequence of any one of the SEQ ID NOs in Table 10 listed as having a degradation Combined Grade of B/B or B, optionally wherein the sequence lacks the HA tag sequence (SEQ ID NO: 50).

100. The dual-specific bifunctional polypeptide of any one of embodiments 90-96, wherein the bifunctional polypeptide does not comprise or consist of an amino acid sequence of any one of the SEQ ID NOs in Table 10 listed as having a degradation Grade of C and/or D for at least one of the target proteins, optionally wherein the sequence lacks the HA tag sequence (SEQ ID NO: 50).

101. The dual-specific bifunctional polypeptide of any one of embodiments 81-86, wherein the dual-specific bifunctional polypeptide comprises or consists of an amino acid sequence of any one of the SEQ ID NOs in Table 11, optionally wherein the western blot shows degradation of one and/or both of the target proteins. 102. The dual-specific bifunctional polypeptide of any one of embodiments 81-101, wherein the URD is not derived from CHIP, optionally wherein the URD does not comprise or consist of CHIP.1, optionally wherein the URD does not comprise or consist of SEQ ID NO: 1836.

103. A polynucleotide encoding a bifunctional polypeptide comprising or consisting of the bifunctional polypeptide of any one of embodiments 81-102.

104. The dual-specific bifunctional polypeptide of any one of embodiments 1-103, wherein the at least one target protein comprises CTNNB1.

105. The dual-specific bifunctional polypeptide of embodiment 104, wherein the CTNNB1 protein is a mutant form of CTNNB1 (SEQ ID NO: 3) comprising one or more mutations selected from: D32Y; D32N; D32V; D32G; S33Y; S33L; S33F; S33C; S33P; G34V; G34E; 135S; H36P; S37C; S37P; S37P and D207G; T41A; T41I; T42_K49del; S45F and Y670X wherein X indicates termination; S45C; S45F; S45P; S45Y; and S45del.

106. The dual-specific bifunctional polypeptide of any one of embodiments 104-105, wherein the bifunctional polypeptide comprises a localization peptide sequence which directs the bifunctional polypeptide to the nucleus of a cell, optionally wherein the localization peptide sequence comprises or consists of a NLS sequence selected from the group of the NLS sequences of MYC, SPOP, Hrp1, SV40, 53BP1, and nucleoplasmin, optionally wherein the localization peptide sequence comprises or consists of a NLS sequence selected from the group of the NLS sequence of MYC, optionally mycNLS and/or 3xmycNLS, SPOP, optionally spNLS and/or spNLS.2, SV40, optionally 3xsvNLS, 53BP1 and Hrp1.

107. The dual-specific bifunctional polypeptide of embodiment 106, wherein the NLS comprises or consists of a sequence selected from any one of SEQ ID NOs: 51, 1896-1909, optionally wherein the NLS comprises or consists of a sequence selected from any one of SEQ ID NOs: 1896-1899, 1901 and 1905.

108. The dual-specific bifunctional polypeptide of any one of embodiments 104107, wherein the URD is the URD of DCAF1, NHLRC1, RNF125, RNF165, and SPOP, optionally NHLRC1, RNF125, RNF165, and SPOP, or a truncation or fragment thereof that retains ubiquitin-proteasome recruiting activity, optionally DCAF1.1, NHLRC1.1, RNF125.2, RNF125.3, RNF125.4, RNF125.5, RNF125.6, RNF165.1, SPOP.2 or SPOP.3, optionally NHLRC1.1, RNF125.2, RNF125.3, RNF125.4, RNF125.5, RNF125.6, RNF165.1, SPOP.2 or SPOP.3.

109. The dual-specific bifunctional polypeptide of any one of embodiment 104-108, wherein the URD comprises or consists of a sequence selected from any one of SEQ ID NOs: 29-43, and 1836-1895, optionally wherein the URD comprises or consists of a sequence selected from any one of SEQ ID NOs: 1837, 1855, 1856, 1858, 1870-1874, and 1880, or 1855, 1856, 1858, 1870-1874, and 1880.

110. The dual-specific bifunctional polypeptide of any one of embodiments 104-109, wherein the first and/or second targeting moiety binds, or binds at least in part, to at least a portion of:
a) the armadillo domain of CTNNB1,
b) amino acids 150-663 of CTNNB1 (SEQ ID NO: 3),
c) amino acids 15-29 of CTNNB1 (SEQ ID NO: 3), and
d) amino acids 249-265, 292-306, 379-390, 415-429, 462-470, or 505-519, or any combination thereof, of CTNNB1 (SEQ ID NO: 3).

111. The dual-specific bifunctional polypeptide of any one of embodiments 104-110, wherein the first and/or second targeting moiety comprises or consists of an endogenous protein binding partner of the target protein, a fragment and/or a derivative thereof.

112. The dual-specific bifunctional polypeptide of any one of embodiments 104-111, wherein the first targeting moiety and/or second targeting moiety is TCF4.1, TCF4.2, TCF4.3, TCF4.4, TCF4.5, TCF4.6, TCF4.7, TCF4.8, TCF4.9, TCF4.10, TCF4.11, TCF4.12, TCF4.13, TCF4.14, TCF4.15, or TCF4.16, or a fragment thereof that is capable of binding to the target protein, optionally TCF4.1, TCF4.2, TCF4.3, TCF4.4, TCF4.5, TCF4.6, TCF4.7, TCF4.8, TCF4.12, TCF4.14, or TCF4.16.

113. The dual-specific bifunctional polypeptide of any one of embodiments 104-112, wherein the first targeting moiety and/or second targeting moiety comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NOs: 2087, 2090, 2091, and 2093-2100.

114. The dual-specific bifunctional polypeptide of any one of embodiments 104-113, wherein the dual-specific bifunctional polypeptide comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NO: 2172-2207 and 2209-2281, optionally any one of SEQ ID NO: 2189-2195, 2202-2206, 2217-2220, 2266-2269 and 2271, optionally wherein the sequence lacks the HA tag sequence (SEQ ID NO: 50).

115. The dual-specific bifunctional polypeptide of any one of embodiments 104-114, wherein the bifunctional polypeptide comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of the SEQ ID NOs in Table 7, optionally excluding any one of the SEQ ID NOs listed as having a degradation Grade of D for at least one of the target proteins, optionally wherein the sequence lacks the HA tag sequence (SEQ ID NO: 50).

116. The dual-specific bifunctional polypeptide of any one of embodiments 104-115, wherein the bifunctional polypeptide comprises or consists of an amino acid sequence of any one of the SEQ ID NOs in Table 7 listed as having a CTNNB1 degradation Grade of A, and/or a CTNNB1 degradation Grade of B, optionally wherein the sequence lacks the HA tag sequence (SEQ ID NO: 50).

117. The dual-specific bifunctional polypeptide of any one of embodiments 104-115, wherein the dual-specific bifunctional polypeptide comprises or consists of an amino acid sequence of any one of the SEQ ID NOs in Table 7 listed as having a degradation Combined Grade of A/A or A, optionally wherein the sequence lacks the HA tag sequence (SEQ ID NO: 50).

118. The dual-specific bifunctional polypeptide of any one of embodiments 104-115, wherein the dual-specific bifunctional polypeptide comprises or consists of an amino acid sequence of any one of the SEQ ID NOs in Table 7 listed as having a degradation Combined Grade of A/B or B/A, optionally wherein the sequence lacks the HA tag sequence (SEQ ID NO: 50).

119. The dual-specific bifunctional polypeptide of any one of embodiments 104-115, wherein the dual-specific bifunctional polypeptide comprises or consists of an amino acid sequence of any one of the SEQ ID NOs in Table 7 listed as having a degradation Combined Grade of B/B or B, optionally wherein the sequence lacks the HA tag sequence (SEQ ID NO: 50).

120. The dual-specific bifunctional polypeptide of any one of embodiments 104-119, wherein the bifunctional polypeptide does not comprise or consist of an amino acid sequence of any one of the SEQ ID NOs in Table 7 listed as having a degradation Grade of C and/or D for at least one of the target proteins, optionally wherein the sequence lacks the HA tag sequence (SEQ ID NO: 50).

121. The dual-specific bifunctional polypeptide of any one of embodiments 104-120, wherein the dual-specific bifunctional polypeptide comprises or consists of an amino acid sequence of any one of the SEQ ID NOs in Table 8, optionally wherein the western blot shows degradation of one and/or both of the target proteins.

122. The bifunctional polypeptide of any one of embodiments 104-121, wherein the URD is not derived from beta-TRCP, optionally wherein the URD does not comprise or consist of bTRCP.1, optionally wherein the URD does not comprise or consist of SEQ ID NOs: 1839.

123. A polynucleotide encoding a bifunctional polypeptide comprising or consisting of the bifunctional polypeptide of any one of embodiments 104-122.

124. The dual-specific bifunctional polypeptide of any one of embodiments 1-123, wherein the at least one target protein comprises PCNA.

125. The dual-specific bifunctional polypeptide of embodiment 124, wherein the bifunctional polypeptide comprises a localization peptide sequence which directs the bifunctional polypeptide to the nucleus of a cell, optionally wherein the localization peptide sequence comprises or consists of a NLS sequence selected from the group of the NLS sequences of MYC, SPOP, Hrp1, SV40, 53BP1, and nucleoplasmin, optionally wherein the localization peptide sequence comprises or consists of a NLS sequence selected from the group of the NLS sequence of MYC, optionally meyNLS and/or 3xmycNLS, SPOP, Hrp1, SV40, optionally svNLS or 3xsvNLS, and nucleoplasmin.

126. The dual-specific bifunctional polypeptide of embodiment 125, wherein the NLS comprises or consists of a sequence selected from any one of SEQ ID NOs: 51, 1896-1909, optionally wherein the NLS comprises or consists of a sequence selected from any one of SEQ ID NOs: 1897-1898, and 1900-1904.

127. The dual-specific bifunctional polypeptide of any one of embodiments 124, wherein the URD is the URD of DCAF1, NHLRC1, RNF125, or SPOP, or a truncation or fragment thereof that retains ubiquitin-proteasome recruiting activity, optionally DCAF1.1, NHLRC1.1, RNF125.2, RNF125.3, RNF125.4, RNF125.5, RNF125.6, SPOP.2 or SPOP.3, or optionally NHLRC1.1 or RNF125.2.

128. The dual-specific bifunctional polypeptide of any one of embodiments 124-127, wherein the URD comprises or consists of a sequence selected from any one of SEQ ID NOs: 29-43, and 1836-1895, optionally wherein the URD comprises or consists of a sequence selected from any one of SEQ ID NOs: 1837, 1855, 1856, 1858, and 1870-1874, optionally SEQ ID NOs: 1858 and 1870.

129. The dual-specific bifunctional polypeptide of any one of embodiments 124-128, wherein the first and/or second targeting moiety binds, or binds at least in part, to at least a portion of amino acids 38-48, 123-129, or 251-257 or any combination thereof, of PCNA (SEQ ID NO: 2152):

130. The dual-specific bifunctional polypeptide of any one of embodiments 124-129, wherein the first and/or second targeting moiety comprises or consists of an endogenous protein binding partner of the target protein, a fragment and/or a derivative thereof.

131. The dual-specific bifunctional polypeptide of any one of embodiments 124-130, wherein the first and/or second targeting moiety comprises or consists of con1.1, con1.2, p21.1, p21.2, p21.3, p21.4, p21.5, p21.6, p21.7, p21.8, p21.9, or a fragment thereof that is capable of binding to the target protein.

132. The dual-specific bifunctional polypeptide of any one of embodiment 124-131, wherein the first and/or second targeting moiety comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NOs: 2102-2112.

133. The dual-specific bifunctional polypeptide of any one of embodiments 124-132, wherein the dual-specific bifunctional polypeptide comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NO: 2202-2285, and 2284-2286, optionally any one of SEQ ID NO: 2202-2206, 2217-2220, 2266-2269, and 2271, optionally wherein the sequence lacks the HA tag sequence (SEQ ID NO: 50).

134. The dual-specific bifunctional polypeptide of any one of embodiments 124-133, wherein the bifunctional polypeptide comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of the SEQ ID NOs in Table 13, optionally excluding any one of the SEQ ID NOs listed as having a degradation Grade of D for at least one of the target proteins, optionally wherein the sequence lacks the HA tag sequence (SEQ ID NO: 50).

135. The dual-specific bifunctional polypeptide of any one of embodiments 124~134, wherein the bifunctional polypeptide comprises or consists of an amino acid sequence of any one of the SEQ ID NOs in Table 13 listed as having a PCNA degradation Grade of A, and/or a PCNA degradation Grade of B, optionally wherein the sequence lacks the HA tag sequence (SEQ ID NO: 50).

136. The dual-specific bifunctional polypeptide of any one of embodiments 124-134, wherein the dual-specific bifunctional polypeptide comprises or consists of an amino acid sequence of any one of the SEQ ID NOs in Table 13 listed as having a degradation Combined Grade of A/A or A, optionally wherein the sequence lacks the HA tag sequence (SEQ ID NO: 50).

137. The dual-specific bifunctional polypeptide of any one of embodiments 124-134, wherein the dual-specific bifunctional polypeptide comprises or consists of an amino acid sequence of any one of the SEQ ID NOs in Table 13 listed as having a degradation Combined Grade of A/B or B/A, optionally wherein the sequence lacks the HA tag sequence (SEQ ID NO: 50).

138. The dual-specific bifunctional polypeptide of any one of embodiments 124-134, wherein the dual-specific bifunctional polypeptide comprises or consists of an amino acid sequence of any one of the SEQ ID NOs in Table 13 listed as having a degradation Combined Grade of B/B or B, optionally wherein the sequence lacks the HA tag sequence (SEQ ID NO: 50).

139. The dual-specific bifunctional polypeptide of any one of embodiments 124-138, wherein the bifunctional polypeptide does not comprise or consist of an amino acid sequence of any one of the SEQ ID NOs in Table 13 listed as having a degradation Grade of C and/or D for at least one of the target proteins, optionally wherein the sequence lacks the HA tag sequence (SEQ ID NO: 50).

140. The dual-specific bifunctional polypeptide of any one of embodiments 124-139, wherein the dual-specific bifunctional polypeptide comprises or consists of an amino acid sequence of any one of the SEQ ID NOs in Table 14, optionally wherein the western blot shows degradation of one and/or both of the target proteins.

141. The dual-specific bifunctional polypeptide of any one of embodiments 124-140, wherein the URD is not derived from SPOP, optionally wherein the URD does not comprise or consist of SPOP.1, SPOP.2, or SPOP.3, optionally wherein the URD does not comprise or consist of a sequence selected from any one of SEQ ID NOs: 1854-1856.

142. A polynucleotide encoding a dual-specific bifunctional polypeptide comprising or consisting of the bifunctional polypeptide of any one of embodiments 124-141.

143. The dual-specific bifunctional polypeptide of any one of embodiments 1-103, wherein the first target protein is c-MYC and the second target protein is c-MYC.

144. The dual-specific bifunctional polypeptide of any one of embodiments 1-80 and 104-123, wherein the first target protein is CTNNB1 and the second target protein is CTNNB1.

145. The dual-specific bifunctional polypeptide of any one of embodiments 1-80 and 124-142, wherein the first target protein is PCNA the second target protein is PCNA.

146. The dual-specific bifunctional polypeptide of any one of embodiments 1-123 wherein the first target protein is c-MYC and the second target protein is CTNNB1, or the first target protein is CTNNB1 and the second target protein is c-MYC.

147. The dual-specific bifunctional polypeptide of any one of embodiments 1-103 and 124-142, wherein the first target protein is c-MYC and the second target protein is PCNA, or wherein the first target protein is PCNA and the second target protein is c-MYC.

148. The dual-specific bifunctional polypeptide of any one of embodiments 1-80, and 104-142, wherein the first target protein is CTNNB1 and the second target protein is PCNA, or wherein the first target protein is PCNA and the second target protein is CTNNB1.

149. The dual-specific bifunctional polypeptide of any one of embodiments 143-148, wherein the dual-specific bifunctional polypeptide comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NO:2172-2207 and 2209-2351, or optionally 2172-2207 and 2209-2286, optionally wherein the sequence lacks the HA tag sequence (SEQ ID NO: 50).

150. A polypeptide comprising or consisting of an amino acid sequence having at least 80%, 85%, 86%, 87%, 88%, 89%, 90%, 91%, 92%, 93%, 94%, 95%, 96%, 97%, 98%, 99%, or 100% sequence identity to 2172-2207 and 2209-2351, or optionally 2172-2207 and 2209-2286, optionally wherein the sequence lacks the HA tag sequence (SEQ ID NO: 50).

151. A polynucleotide encoding for a polypeptide having at least 80%, 85%, 86%, 87%, 88%, 89%, 90%, 91%, 92%, 93%, 94%, 95%, 96%, 97%, 98%, 99%, or 100% sequence identity to the dual-specific bifunctional polypeptide of any one of embodiments 1-150.

152. A pharmaceutical composition comprising the polypeptide, dual-specific bifunctional polypeptide, or polynucleotide of any one of embodiments 1-151 and one or more pharmaceutically acceptable excipients, carriers, or diluents.

153. The pharmaceutical composition embodiment 152, wherein the composition is formulated for intravenous, intraperitoneal, intra-arterial, subcutaneous, intramuscular, intrathecal, intratumoral, inhalation, or intracranial administration.

154. The polypeptide, dual-specific bifunctional polypeptide, polynucleotide, or pharmaceutical composition of any one of embodiments 1-153 for use in the treatment of a cancer in a patient in need thereof.

155. The polypeptide, dual-specific bifunctional polypeptide, polynucleotide, or pharmaceutical composition of any one of embodiments 1-153 for use in the manufacture of a medicament, optionally for the treatment of cancer.

156. A method of treating a subject comprising administering the polypeptide, dual-specific bifunctional polypeptide, polynucleotide, or pharmaceutical composition of any one of embodiments 1-155 to a subject in need thereof, optionally wherein the subject has a cancer.

157. A method of reducing the amount of a target protein in a cell comprising contacting the cell with the polypeptide, dual-specific bifunctional polypeptide, polynucleotide, or pharmaceutical composition of any one of embodiments 1-155, optionally wherein the target protein is selected from MYC, (optionally c-MYC), CTNNB1, and/or PCNA.

158. The method of embodiment 156 or 157, wherein said cell is in a subject, and said contacting comprises administering the polypeptide, dual-specific bifunctional polypeptide, polynucleotide, or pharmaceutical composition of any one of embodiments 1-153 to the subject, optionally wherein the subject has cancer.

159. The method of embodiment 158, wherein the administering is intravenous, intraperitoneal, intra-arterial, subcutaneous, intramuscular, intrathecal, intratumoral, inhalation, or intracranial administration.

160. The method of any one of embodiments 156 or 157, wherein the cell is ex vivo, and said contacting comprises contacting the polypeptide, dual-specific bifunctional polypeptide, polynucleotide, or pharmaceutical composition of any one of embodiments 1-153 to the cell ex vivo, optionally in an adoptive cell therapy.

161. The method of embodiment 160, further comprising administering the cell to a subject after the contacting step, optionally wherein the subject is also the source of the cell.

162. The polypeptide, dual-specific bifunctional polypeptide, polynucleotide, pharmaceutical composition, or method of any one of the preceding embodiments, wherein the polypeptide or dual-specific bifunctional polypeptide promotes proteasome-mediated degradation of the target protein when measured in one or more assays described in the Exemplary Assays for assessing proteasomal-mediated degradation, for example, by HiBiT tag assay and/or Western blot, optionally wherein the proteasome-mediated degradation is inhibited when the cells comprising the target protein and the dual-specific bifunctional polypeptide are treated with a proteasome inhibitor.

163. The dual-specific bifunctional polypeptide, polypeptide, polynucleotide, pharmaceutical composition, or method of embodiment 162, wherein treatment of the cells comprising the target protein and the polypeptide or dual-specific bifunctional polypeptide with a proteasome inhibitor reduces the amount of degradation of the target protein by at least 50%, 60%, 70%, 80%, 90%, 95% or 100% as compared to cells not treated with the proteasome inhibitor.

164. The dual-specific bifunctional polypeptide, polypeptide, polynucleotide, pharmaceutical composition, or method of any one of the preceding embodiments, wherein the polypeptide or dual-specific bifunctional polypeptide does not comprise a URD derived from CHIP, SPOP, TRIM21 or beta-TRCP.

### BRIEF DESCRIPTION OF THE DRAWINGS

In addition to the features described above, additional features and variations will be readily apparent from the following descriptions of the drawings and exemplary embodiments. It is to be understood that these drawings depict typical embodiments and are not intended to be limiting in scope.

FIGS. 1A and 1B depict embodiments of a schematic describing proteasome-mediated degradation of a target protein using a dual-specific bifunctional polypeptide, which comprises a first and second targeting domain specific for a first and second target protein and a URD comprising an E3 ligase or component, fragment, or truncation thereof that recruits the ubiquitination complex to ubiquitinate one (not shown) or both of the target protein(s) for proteasomal degradation. FIG. 1A depicts an embodiment of a “contralateral” configuration in which the first and second targeting domains are located on either side of the URD. FIG. 1B depicts and embodiment of a “tandem” configuration in which the first and second targeting domains are located on the same side (both on the N-terminal side or both on the C-terminal side of the URD).

FIGS. 2A and 2B depict embodiments of a schematic for different ordering combinations of the components that comprise the dual-specific bifunctional polypeptides of some embodiments disclosed herein. FIG. 2A depicts embodiments of a “contralateral” configuration in which the dual-specific bifunctional polypeptides comprise a first targeting moiety (target binding domain) (N-TBD or C-TBD) that is capable of binding to at least one target protein; a second targeting moiety (C-TBD or N-TBD) that is capable of binding to at least one target protein; and a ubiquitin-proteasome system recruiting domain (URD). FIG. 2B depicts embodiments of a “tandem” configuration in which the dual-specific bifunctional polypeptide is in a “tandem” configuration in which the first and second target binding domains are both on the same side of the URD, i.e., both are on the C-terminal side of the URD, or both are on the N-terminal side of the URD. In some embodiments the dual-specific bifunctional polypeptide further comprises one or more linker peptides. In some embodiments when in a “contralateral” configuration the dual-specific bifunctional polypeptide further comprises a first linker peptide (N-linker or C-linker), wherein the first linker peptide is positioned between the first targeting moiety and the URD; and/or a second linker peptide (C-linker or N-linker), wherein the second linker peptide is positioned between the second targeting moiety and the URD. In some embodiments, there is no linker between the first and/or second targeting moiety and the URD. In some embodiments when a “tandem” configuration is used (see, e.g., FIG. 2B), a linker peptide (a first or a second linker peptide) is positioned between the two target binding domains, and/or a target binding domain and the URD. In some embodiments of the tandem configuration one or both of the linkers is not present (not shown). The linker(s), if present, may provide flexibility in positioning between the URD and target binding moiety. In some embodiments, the dual-specific bifunctional polypeptides may further comprise a localization sequence, (e.g., a nuclear localization signal (NLS)), which may be at the N-terminus, C-terminus, or both the N-terminus and C-terminus, or internally (not shown) within the bifunctional polypeptide (e.g., if the URD and/or target binder comprises an NLS within its sequence), if the target protein localizes to the nucleus of a cell. In some embodiment, the localization sequence(s) (e.g., N-NLS, and/or C-NLS) may be separated from other components by a linker sequence (not shown).

FIG. 3 depicts an embodiment of subcellular localization of SPOP variant URD mono-specific bifunctional polypeptides to the nucleus and the cytoplasm.

FIG. 4 depicts an embodiment of subcellular localization of NHLRC1 containing mono-specific bifunctional polypeptides with and without an exogenous NLS sequence.

FIG. 5 depicts an embodiment of subcellular localization of V_{L}12.3 containing mono-specific bifunctional polypeptides with URDs containing endogenous NLS or NES signals.

FIG. 6 is a table disclosing a summary of an embodiment of an experiment examining expression of various exemplary dual-specific bifunctional polypeptides. Provided is a description of elements listed in a N- to C-terminal direction, a summary of the observed subcellular localization of HA staining and the respective SEQ ID NOs.

FIG. 7A-C depicts an embodiment of HA staining of the dual-specific bifunctional polypeptides disclosed in FIG. 6.

FIG. 8A-8H depicts an embodiment of various exemplary dual-specific bifunctional polypeptides listing the target proteins (“Targets”), description of elements listed in a N- to C-terminal direction, and respective SEQ ID NOs.

FIG. 9A-9C is a table disclosing a summary of an embodiment of an experiment examining degradation of HiBiT™-labeled target proteins by dual-specific bifunctional polypeptides. Provided is the target proteins, a description of the elements of the dual-specific bifunctional polypeptide listed in a N- to C-terminal direction, the Grade of degradation observed for each target, the combined Grade of degradation, and the respective SEQ ID NOs of the dual-specific bifunctional polypeptide.

FIG. 10A-10B is a table disclosing a summary of an embodiment of an experiment examining degradation of target proteins by dual-specific bifunctional polypeptides using a western blot. Provided is the target proteins, a description of the elements of the dual-specific bifunctional polypeptide listed in a N- to C-terminal direction, and the respective SEQ ID NOs of the dual-specific bifunctional polypeptide used in the experiment.

FIG. 11 is a table disclosing a summary of an embodiment of an experiment examining degradation of target proteins CTNNB1 and MYC by dual-specific bifunctional polypeptides using a western blot. Provided is the target proteins, a description of the elements of the dual-specific bifunctional polypeptide listed in a N- to C-terminal direction, and the respective SEQ ID NOs of the dual-specific bifunctional polypeptide used in the experiment.

FIG. 12 is an embodiment of western blots disclosing the results of the degradation experiment using the dual-specific bifunctional polypeptides disclosed in FIG. 11.

FIG. 13 is a table disclosing a summary of an embodiment of an experiment examining degradation of target proteins CTNNB1 and PCNA by dual-specific bifunctional polypeptides using a western blot. Provided is the target proteins, a description of the elements of the dual-specific bifunctional polypeptide listed in a N- to C-terminal direction, and the respective SEQ ID NOs of the dual-specific bifunctional polypeptide used in the experiment.

FIG. 14 and FIG. 15 are embodiments of western blots disclosing the results of the degradation experiment using the dual-specific bifunctional polypeptides disclosed in FIG. 13.

FIG. 16 is a table disclosing a summary of an embodiment of an experiment examining degradation of target proteins CTNNB1 and PCNA by dual-specific bifunctional polypeptides using a western blot. Provided is the target proteins, a description of the elements of the dual-specific bifunctional polypeptide listed in a N- to C-terminal direction, and the respective SEQ ID NOs of the dual-specific bifunctional polypeptide used in the experiment.

FIG. 17 is an embodiment of western blots disclosing the results of the degradation experiment using the dual-specific bifunctional polypeptides disclosed in FIG. 16.

FIG. 18 is a table disclosing a summary of an embodiment of an experiment examining degradation of target proteins CTNNB1 and PCNA by dual-specific bifunctional polypeptides using a western blot. Provided is the target proteins, a description of the elements of the dual-specific bifunctional polypeptide listed in a N- to C-terminal direction, and the respective SEQ ID NOs of the dual-specific bifunctional polypeptide used in the experiment.

FIG. 19 is an embodiment of western blots disclosing the results of the degradation experiment using the dual-specific bifunctional polypeptides disclosed in FIG. 18.

### DETAILED DESCRIPTION OF THE DISCLOSURE

The ubiquitin-proteasome system is the major and essential mechanism by which eukaryotic cells regulate protein abundance and clear misfolded or damaged proteins through proteolytic degradation. This regulation is vital for many cellular functions, such as regulating the cell cycle and gene expression. Proteins are marked for degradation and recognized by the proteasomal complex when they are polyubiquitinated at lysine residues. This polyubiquitination is mediated by recognition of the target proteins by a E3 ubiquitin ligase, which catalyzes the transfer of a ubiquitin subunit from an E2 ubiquitin-conjugating enzyme to the protein target. As ubiquitin also contains ubiquitination sites, the protein target can be polyubiquitinated, thereby marking it for degradation by the proteasome. It is estimated that there are over 600 unique E3 ubiquitin ligases encoded by the human genome, each having varying specificities to target proteins. Therefore, each E3 ubiquitin ligase generally comprises a substrate recognition domain specific for one or more targets and a ubiquitin-proteasome system recruiting domain (URD) that binds to an E2 ubiquitin-conjugating enzyme to enable ubiquitination of the target.

As disclosed herein, the ubiquitin-proteasome system may be exploited for the directed degradation of desired protein targets. For example, many diseases are associated with the abnormal function and/or expression of certain proteins (e.g., cancer caused by dysfunctional expression or localization of oncogenes). Degradation of said proteins can have a therapeutic effect in treating associated diseases, and the ubiquitin-proteasome system offers a naturally occurring process for effecting said degradation. Furthermore, degradation of disease-associated proteins may be more effective than inhibition of the protein, for example, using a small molecule or antibody composition, as in these cases, the protein is still present within the cell and may perform various biological functions at interfaces other than the inhibited domain, for example scaffolding functions, or contribute to pathology as a result of accumulation, aggregation or mislocalization.

Several groups have reported on the structure and preclinical activity of various macromolecule degrader formats, including fusion proteins comprising a full-length or truncated E3 ligase and macromolecular targeting constructs. However, general design principles for such macromolecule degraders are not yet well established, comparatively few of the hundreds of naturally occurring E3 ligases have been explored in this context, and degradation of intracellular targets using macromolecule degraders has been demonstrated for only a limited number of target proteins. Furthermore, there is little precedent for the optimization of drug-like properties for such molecules, and none have yet advanced to the stage of clinical testing. Examples of previous macromolecule degraders have been explored in: Wang et al., “The state of the art of PROTAC technologies for drug discovery,” Eur J. Med. Chem. (2022) 235:114290; Lim et al., “bioPROTACs as versatile modulators of intracellular therapeutic targets including proliferating cell nuclear antigen (PCNA),” PNAS (2020) 117(11): 5791-5800; Hatakeyama et al. “Targeted destruction of c-Myc by an engineered ubiquitin ligase suppresses cell transformation and tumor formation,” Cancer Res. (2005) 65(17):7874-9; Portnoff et al. “Ubiquibodies, synthetic E3 ubiquitin ligases endowed with unnatural substrate specificity for targeted protein silencing,” J. Biol. Chem. (2014) 289(11):7844-55; Liao et al. “A PROTAC peptide induces durable β-catenin degradation and suppresses Wnt-dependent intestinal cancer,” Cell Discov. (2020) 6:35; Liu et al. Targeted degradation of b-catenin by chimeric F-box fusion proteins. Biochem. and Biophys. Res. Comm. (2004) 313:1023-1029; Cong et al., “A protein knockdown strategy to study the function of β-catenin in tumorigenesis,” BMC Molecular Bio. (2003) 4:10; and Shu et al. “Eradication of pathogenic β-catenin by Skp1/Cullin/F box ubiquitination machinery,” PNAS (2003) 100(22)12729-12734; each of which is hereby expressly incorporated by reference in its entirety. Localization of degraders to the correct subcellular compartment may be important for the ability to degrade a target protein, e.g., if the target is localized to the nucleus, or if degradation of target protein only in the nucleus but not cytoplasm is desired. It is noteworthy that apparently none of these references address, or even consider, this potentially important aspect of degrader design. Further, it is noted that none of these degrader formats include dual-specific degraders as disclosed herein.

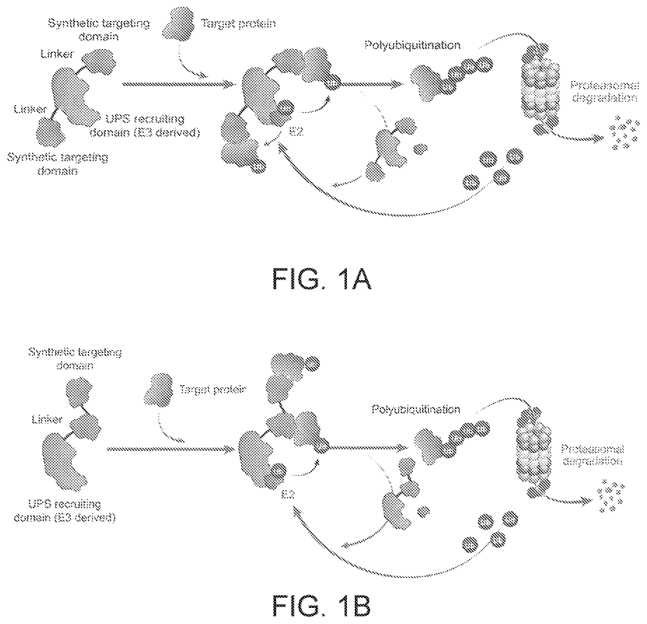

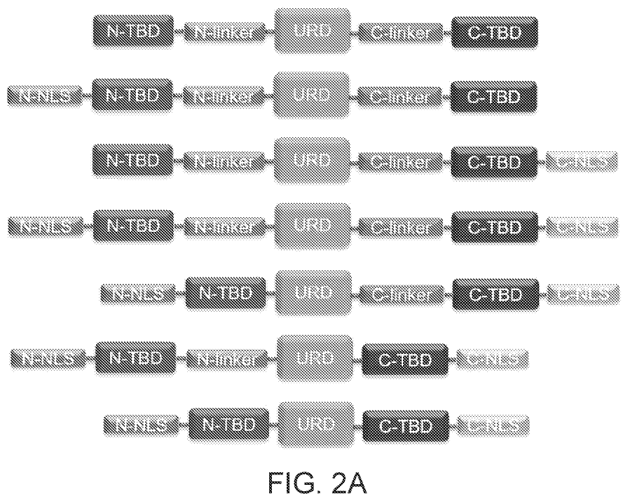

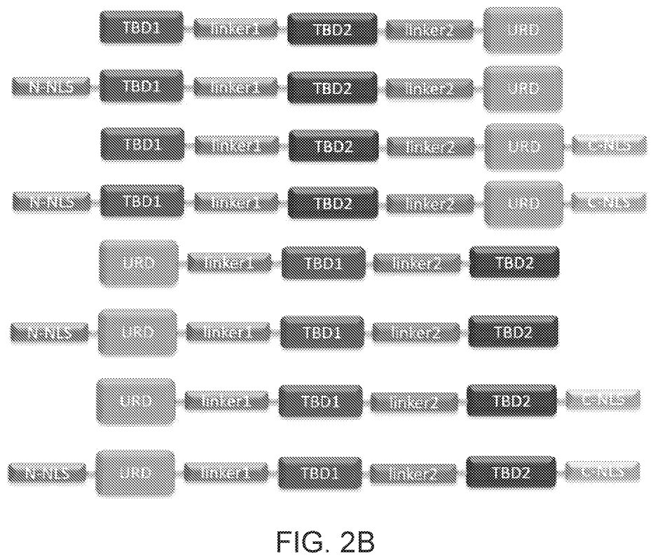

Accordingly, provided herein are dual-specific bifunctional polypeptides that promote proteasome-mediated degradation of a target protein, and uses thereof, such as for the treatment of a disease. These dual-specific bifunctional polypeptides generally comprise a first component that is able to bind to a first target protein with specificity, a second component that is able to bind to a second target protein with specificity, and a third component that is able to recruit a ubiquitination complex to mark the target protein for proteasomal degradation by ubiquitination. An exemplary schematic for the function of the dual-specific bifunctional polypeptides disclosed herein may be seen in FIGS. 1A (contralateral) and 1B (tandem). FIGS. 2A and 2B depicts some non-limiting embodiments of dual-specific bifunctional polypeptides with various combinations of targeting moieties and URD, and optionally localization sequences (e.g., NLS) and linkers (the depiction is in the conventional N-terminal to C-terminal (left to right) orientation). In some embodiments, the first and second targeting domains are on either side of the URD (see, e.g., FIG. 2A) in a “contralateral” configuration. In some embodiments the first and second target binding domains are on the same side of the URD (both on the N-terminal side, or both on the C-terminal side) (see, e.g., FIG. 2B) in a “tandem” configuration. In some embodiments shown in FIG. 2A or 2B, a localization sequence (e.g., NLS) is present at both the N- and C-terminal ends. In some embodiments not shown each of the localization signals (e.g., NLS) shown can represent one, two, three or more copies of the same or a different NLS sequence. In some embodiment, the localization sequence(s) (e.g., N-NLS, and/or C-NLS) may be separated from other components by a linker sequence (not shown).

In some embodiments provided herein are dual-specific bifunctional polypeptides which are engineered (e.g., by modification of an endogenous localization peptide sequence, by adding localization peptide sequences, and/or by selecting a component which has an endogenous localization sequence) to direct the bifunctional polypeptide to a particular subcellular compartment, e.g., the cytoplasm, the nucleus, or both. Further, in some embodiments provided herein are polypeptides comprising or consisting of a portion derived from a an E3 ubiquitin ligase or viral homolog thereof. In some embodiments, the portion has ubiquitin-proteasome recruiting activity and may be designated as a ubiquitin recruiting domain (URD). In some embodiments provided herein are dual-specific bifunctional polypeptides comprising these portions.

### Portions/URDs of E3 Ubiquitin Ligases and Viral Homologs

Disclosed herein are polypeptides comprising or consisting of a portion derived from a protein selected from the group consisting of a RING family E3 ubiquitin ligase, a cullin family E3 ubiquitin ligase, a homologous to E6AP carboxyl terminus (HECT) family E3 ubiquitin ligase, and a viral homolog of an E3 ubiquitin ligase. In some embodiments the viral homolog is VIF of HIV-1. In some embodiments, the portion comprises an amino acid substitution (optionally a conservative substitution), addition, and/or deletion to the protein. In some embodiments, the deletion is not an end terminal deletion or truncation of the protein. In some embodiments, the polypeptide and/or the portion is a non-natural polypeptide. In some embodiments, the portion has ubiquitin-proteasome recruiting activity. In some embodiments, the portion is a ubiquitin-proteasome system recruiting domain (URD) that has ubiquitin-proteasome recruiting activity. In some embodiments the portion of E3 ligase or viral homolog is fused to a polypeptide. In some embodiments, the fusion polypeptide comprises a linker, localization sequence (e.g., NLS), first and/or second targeting moiety, and/or other polypeptide disclosed herein.

In some embodiments, the portion is derived from an E3 ubiquitin ligase selected from the group consisting of CHIP, DCAF1, E6AP, FBXW7-alpha (also referred to herein as “FBXW7a”), FBXW7-beta (also referred to herein as “FBXW7b”), Keap1, NHLRC1, RNF4, RNF6, RNF11, RNF12, RNF20, RNF25, RNF111, RNF114, RNF115, RNF125, RNF128, RNF138, RNF149, RNF152, RNF165, RNF166, RNF182, SPOP, beta-TRCP, TRIM21, TRIM32, VIF, ZNRF1, ZNRF4, and CBL-b (Y363E). Table 1 below discloses representative sequences of the preceding. In some embodiments, the portion is selected from the group consisting of CBLb.1, CHIP.1, DCAF1.1, E6AP.1, FBXW7a.1, FBXW7a.2, FBXW7a.3, FBXW7a.4, FBXW7a.5, FBXW7a.6, FBXW7a.7, FBXW7a.8, FBXW7a.9, FBXW7b.1, FBXW7b.2, FBXW76.3, FBXW7b.4, Keap1.1, NHLRC1.1, NHLRC1.2, NHLRC1.3, NHLRC1.4, NHLRC1.5, RNF4.1, RNF6.1, RNF6.2, RNF11.1, RNF12.1, RNF12.2, RNF20.1, RNF25.1, RNF111.1, RNF114.1, RNF115.1, RNF125.1, RNF125.2, RNF125.3, RNF125.4, RNF125.5, RNF125.6, RNF125.7, RNF128.1, RNF138.1, RNF149.1, RNF152.1, RNF165.1, RNF166.1, RNF182.1, RNF182.2, SPOP.1, SPOP.2, SPOP.3, bTRCP.1, TRIM21.1, TRIM21.2, TRIM21.3, TRIM32.1, VIF.1, ZNRF1.1, and ZNRF4.1. Table 2 below discloses representative sequences of the preceding.

In some embodiments, the portion is derived from a monomeric RING family E3 ligase. In some embodiments, the portion is derived from a monomeric RING family E3 ligase selected from the group consisting of NHLRC1, RNF11, RNF111, RNF114, RNF115, RNF12, RNF125, RNF128, RNF138, RNF149, RNF152, RNF165, RNF166, RNF182, RNF20, RNF25, RNF4, RNF6, ZNRF1, ZNRF4, and CBL-b (Y363E). In some embodiments, the portion is derived from a monomeric RING family E3 ligase selected from the group consisting of RNF125, NHL, RC1, RNF4, RNF6, RNF12, RNF138 and ZNRF1. In some embodiments, the portion is selected from the group consisting of NHLRC1.1, NHLRC1.2, NHLRC1.3, NHLRC1.4, NHLRC1.5, RNF11.1, RNF111.1, RNF114.1, RNF115.1, RNF12.1, RNF12.2, RNF125.1, RNF125.2, RNF125.3, RNF125.4, RNF125.5, RNF125.6, RNF125.7, RNF128.1, RNF138.1, RNF149.1, RNF152.1, RNF165.1, RNF166.1, RNF182.1, RNF182.2, RNF20.1, RNF25.1, RNF4.1, RNF6.1, RNF6.2, ZNRF1.1, ZNRF4.1, and CBLb.1. In some embodiments, the portion is selected from the group consisting of NHLRC1.1, NHLRC1.2, NHLRC1.3, NHLRC1.4, NHLRC1.5, RNF125.1, RNF125.2, RNF125.3, RNF125.4, RNF125.5, RNF125.6, RNF125.7, RNF4.1, RNF6.1, RNF6.2, RNF12.1, RNF12.2, RNF138.1, and ZNRF1.1.

In some embodiments, the portion is derived from a cullin family E3 ligase. In some embodiments, the portion is derived from a cullin family E3 ligase selected from the group consisting of DCAF1, beta-TRCP, FBXW7-alpha, FBXW7-beta, Keap1, and SPOP. In some embodiments, the portion is derived from a cullin family E3 ligase selected from the group consisting of beta-TRCP, FBXW7-alpha, and FBXW7-beta. In some embodiments, the portion is selected from the group consisting of DCAF1.1, bTRCP.1, FBXW7a.1, FBXW7a.2, FBXW7a.3, FBXW7a.4, FBXW7a.5, FBXW7a.6, FBXW7a.7, FBXW7a.8, FBXW7a.9, FBXW7b.1, FBXW7b.2, FBXW7b.3, FBXW7b.4, Keap1.1, SPOP.1, SPOP.2, and SPOP.3.

In some embodiments, the number of amino acids in the portion is, is about, or is less than, 80, 75, 70, 65, 60, 65, 50, 45, 40, 35, 30, 25, 20, 15, 10, 5, 4, 3, 2, or 1%, of the number of amino acids in the wild-type full-length protein (e.g. sequences disclosed in Table 1), or a range defined by any two of the preceding values, optionally 15-80, 15-50, 25-80, 25-50, 30-60, 1-80, 2-80, 1-70, 2-70, 2-65%. In some embodiments, the number of amino acids in the portion is, or is about 15-50% of the number of amino acids in the wild-type full-length protein. In some embodiments, the number of amino acids in the portion is, or is about, 2-61% of the number of amino acids in the wild-type full-length protein. In some embodiments, the portion is, is about, is less than or equal to 370, 369, 368, 367, 366, 365, 364, 363, 362, 361, 360, 300, 250, 200, 175, 170, 165, 160, 155, 150, 145, 140, 135, 130, 129, 128, 127, 126, 125, 124, 123, 120, 115, 110, 105, 100, 95, 90, 85, 80, 75, 70, 65, 60, 55, 50, 47, 46, 45, 44, 43, 42, 41, 40, 39, 38, 37, 36, 35, 34, 33, 32, 31, 30, 25, or 20 amino acids long, or a range defined by any two of the preceding values. In some embodiments, the portion is, or is about, 20-370, 20-150, 20-100, 20-80, 30-150, 30-100, 50-150 amino acids long. In some embodiments, the portion is, or is about, 50-100 or 20-100 amino acids long. In some embodiments, the portion is, or is about, 40-130 or 44-126 amino acids long.

In some embodiments, the portion comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NOs: 29-43 and 1836-1895, optionally SEQ ID NOs: 29-43, optionally wherein the portion has ubiquitin-proteasome recruiting activity. In some embodiments, the portion comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NOs: 1858-1888 and 1893-1895, optionally wherein the portion has ubiquitin-proteasome recruiting activity. In some embodiments, the portion comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NOs: 1858-1862, 1867-1875, 1877, 1886-1888, and 1893, optionally wherein the portion has ubiquitin-proteasome recruiting activity. In some embodiments, the portion consists of an amino acid sequence selected from any one of SEQ ID NOs: 1858-1862, 1867-1875, 1877, 1886-1888, and 1893. In some embodiments where the amino acid sequence differs from a recited reference sequence, the difference is the result of the inclusion of one or more modifications relative to the reference sequence. In some embodiments, the one or more modifications comprise a substitution, insertion and/or deletion. In some embodiments, the substitution is a conservative substitution. In some embodiments, the % sequence identity is calculated over the entirety of the reference sequence.

In some embodiments, the portion comprises an amino acid substitution, addition, and/or deletion to the protein. In some embodiments, the deletion is not an end terminal deletion or truncation of the protein. In some embodiments, the polypeptide and/or the portion is a non-natural polypeptide. In some embodiments, the portion is a ubiquitin-proteasome system recruiting domain (URD) that has ubiquitin-proteasome recruiting activity.

In some embodiments, disclosed herein are dual-specific bifunctional polypeptides comprising the polypeptide comprising or consisting of the portion derived from a protein selected from the group consisting of a RING family E3 ubiquitin ligase, a cullin family E3 ubiquitin ligase, a homologous to E6AP carboxyl terminus (HECT) family E3 ubiquitin ligase, and a viral homolog of an E3 ubiquitin ligase, as described in the paragraphs above and elsewhere herein, wherein the dual-specific bifunctional polypeptide comprises: a) the portion of the protein and, b) a first and second targeting moiety (also referred to as a target “binder” or “binding moiety”) that is capable of binding to a first and a second target protein, optionally the same target protein, optionally an intracellular target protein(s). In some embodiment the first and/or second targeting moiety binds selectively or specifically to the target protein(s).

In some embodiments, disclosed herein are dual-specific bifunctional polypeptides that promote proteasome-mediated degradation of a target protein. In some embodiments, the dual-specific bifunctional polypeptide comprises: a) a first and second targeting moiety (also referred to as a target “binder” or “binding moiety”) that is capable of binding to first and a second target protein, optionally the same target protein, optionally an intracellular target protein(s), b) a ubiquitin-proteasome system recruiting domain (URD). In some embodiments, proximity of the dual-specific bifunctional polypeptide to the target protein(s) through binding of the targeting moiety induces ubiquitination of the target protein via the URD, thereby promoting proteasome-mediated degradation of the target protein(s). In some embodiments one or both of the target protein(s) is an intracellular target protein. In some embodiments, the dual-specific bifunctional polypeptide that promotes proteasome-mediated degradation of a target protein(s), comprises or consisting of the polypeptide of comprising or consisting of the portion derived from a protein selected from the group consisting of a RING family E3 ubiquitin ligase, a cullin family E3 ubiquitin ligase, a homologous to E6AP carboxyl terminus (HECT) family E3 ubiquitin ligase, and a viral homolog of an E3 ubiquitin ligase, as described in the paragraphs above and elsewhere herein. In some embodiments of the dual-specific bifunctional polypeptide that promotes proteasome-mediated degradation of a target protein(s), the URD consists of the URD derived from a protein selected from the group consisting of a RING family E3 ubiquitin ligase, a cullin family E3 ubiquitin ligase, a homologous to E6AP carboxyl terminus (HECT) family E3 ubiquitin ligase, and a viral homolog of an E3 ubiquitin ligase, as described in the paragraphs above and elsewhere herein. In some embodiment the first and/or second targeting moiety binds selectively or specifically to the target protein(s).

Any combination of the targeting moieties and the URD explored herein may be used in the construction of the dual-specific bifunctional polypeptide.

TABLE 1
Representative E3 ligases and viral
homologs for derivation of portions

| SEQ ID | | Uniprot | Orientation |
|---|---|---|---|
| NO | E3 Ligase | ID | (C-N) |
| 14 | CHIP human full length | Q9UNE7 | SRD-URD |
| 15 | RNF114 human full length | Q9Y508 | URD-SRD |
| 16 | RNF125 human full length | Q96EQ8 | URD-SRD |
| 17 | RNF138 human full length | Q8WVD3 | URD-SRD |
| 18 | RNF166 human full length | Q96A37 | URD-SRD |
| 19 | NHLRC1 human full length | Q6VVB1 | URD-SRD |
| 20 | CBL-b (Y363E) human full length | Q13191 | SRD-URD |
| 21 | TRIM21 human full length | P19474 | URD-SRD |
| 22 | E6AP human full length | Q05086 | SRD-URD |
| 23 | FBXW7 alpha human full length | Q969H0 | URD-SRD |
| 24 | FBXW7 beta human full length | Q969H0 | URD-SRD |
| 25 | beta-TRCP human full length | Q9Y297 | URD-SRD |
| 26 | Keap1 human full length | Q14145 | URD-SRD |
| 27 | SPOP human full length | O43791 | SRD-URD |
| 28 | DCAF1 human full length | Q9Y4B6 | URD-SRD |
| 2153 | RNF4 human full length | P78317 | SRD-URD |
| 2154 | RNF6 human full length | Q9Y252 | SRD-URD |
| 2155 | RNF11 human full length | Q9Y3C5 | SRD-URD |
| 2156 | RNF12 human full length | Q9NVW2 | SRD-URD |
| 2157 | RNF20 human full length | Q5VTR2 | SRD-URD |
| 2158 | RNF25 human full length | Q96BH1 | URD-SRD |
| 2159 | RNF111 human full length | Q6ZNA4 | SRD-URD |
| 2160 | RNF115 human full length | Q9Y4L5 | SRD-URD |
| 2161 | RNF128 human full length | Q8TEB7 | SRD-URD |
| 2162 | RNF149 human full length | Q8NC42 | SRD-URD |
| 2163 | RNF152 human full length | Q8N8N0 | URD-SRD |
| 2164 | RNF165 human full length | Q6ZSG1 | SRD-URD |
| 2165 | RNF182 human full length | Q8N6D2 | URD-SRD |
| 2166 | TRIM32 human full length | Q13049 | URD-SRD |
| 2167 | VIF HIV-1 full length | P12504 | SRD-URD |
| 2168 | ZNRF1 human full length | Q8ND25 | SRD-URD |
| 2169 | ZNRF4 human full length | Q8WWF5 | SRD-URD |

TABLE 2
Exemplary Portions / URDs derived
from E3 ligases and viral homologs

| | | | Uniprot ID of |
|---|---|---|---|
| SEQ ID NO | Name | E3 Family | Parental Protein |
| 1836 | CHIP.1 | U-box | Q9UNE7 |
| 1837 | DCAF1.1 | H-box | Q9Y4B6 |
| 1838 | E6AP.1 | HECT | Q05086 |
| 1839 | bTRCP.1 | F-box/Cul1 | Q9Y297 |
| 1840 | FBXW7a.1 | F-box/Cul1 | Q969H0 |
| 1841 | FBXW7a.2 | F-box/Cul1 | Q969H0 |
| 1842 | FBXW7a.3 | F-box/Cul1 | Q969H0 |
| 1843 | FBXW7a.4 | F-box/Cul1 | Q969H0 |
| 1844 | FBXW7a.5 | F-box/Cul1 | Q969H0 |
| 1845 | FBXW7a.6 | F-box/Cul1 | Q969H0 |
| 1846 | FBXW7a.7 | F-box/Cul1 | Q969H0 |
| 1847 | FBXW7a.8 | F-box/Cul1 | Q969H0 |
| 1848 | FBXW7a.9 | F-box/Cul1 | Q969H0 |
| 1849 | FBXW7b.1 | F-box/Cul1 | Q969H0 |
| 1850 | FBXW7b.2 | F-box/Cul1 | Q969H0 |
| 1851 | FBXW7b.3 | F-box/Cul1 | Q969H0 |
| 1852 | FBXW7b.4 | F-box/Cul1 | Q969H0 |
| 1853 | Keap1.1 | BTB/Cul3 | Q14145 |
| 1854 | SPOP.1 | BTB/Cul3 | O43791 |
| 1855 | SPOP.2 | BTB/Cul3 | O43791 |
| 1856 | SPOP.3 | BTB/Cul3 | O43791 |
| 1857 | VIF.1 | BC-Box/Cul5 | P12504 |
| 1858 | NHLRC1.1 | RING | Q6VVB1 |
| 1859 | NHLRC1.2 | RING | Q6VVB1 |
| 1860 | NHLRC1.3 | RING | Q6VVB1 |
| 1861 | NHLRC1.4 | RING | Q6VVB1 |
| 1862 | NHLRC1.5 | RING | Q6VVB1 |
| 1863 | RNF11.1 | RING | Q9Y3C5 |
| 1864 | RNF111.1 | RING | Q6ZNA4 |
| 1865 | RNF114.1 | RING | Q9Y508 |
| 1866 | RNF115.1 | RING | Q9Y4L5 |
| 1867 | RNF12.1 | RING | Q9NVW2 |
| 1868 | RNF12.2 | RING | Q9NVW2 |
| 1869 | RNF125.1 | RING | Q96EQ8 |
| 1870 | RNF125.2 | RING | Q96EQ8 |
| 1871 | RNF125.3 | RING | Q96EQ8 |
| 1872 | RNF125.4 | RING | Q96EQ8 |
| 1873 | RNF125.5 | RING | Q96EQ8 |
| 1874 | RNF125.6 | RING | Q96EQ8 |
| 1875 | RNF125.7 | RING | Q96EQ8 |
| 1876 | RNF128.1 | RING | Q8TEB7 |
| 1877 | RNF138.1 | RING | Q8WVD3 |
| 1878 | RNF149.1 | RING | Q8NC42 |
| 1879 | RNF152.1 | RING | Q8N8N0 |
| 1880 | RNF165.1 | RING | Q6ZSG1 |
| 1881 | RNF166.1 | RING | Q96A37 |
| 1882 | RNF182.1 | RING | Q8N6D2 |
| 1883 | RNF182.2 | RING | Q8N6D2 |
| 1884 | RNF20.1 | RING | Q5VTR2 |
| 1885 | RNF25.1 | RING | Q96BH1 |
| 1886 | RNF4.1 | RING | P78317 |
| 1887 | RNF6.1 | RING | Q9Y252 |
| 1888 | RNF6.2 | RING | Q9Y252 |
| 1889 | TRIM21.1 | RING | P19474 |
| 1890 | TRIM21.2 | RING | P19474 |
| 1891 | TRIM21.3 | RING | P19474 |
| 1892 | TRIM32.1 | RING | Q13049 |
| 1893 | ZNRF1.1 | RING | Q8ND25 |
| 1894 | ZNRF4.1 | RING | Q8WWF5 |
| 1895 | CBLb.1 | RING | Q13191 |

In some embodiments, the component of the dual-specific bifunctional polypeptide that is capable of recruiting the ubiquitination complex is generally composed of a ubiquitin-proteasome system recruiting domain (URD). In some embodiments, the URD may be derived from an E3 ubiquitin ligase, such as a human E3 ubiquitin ligase. However, in some embodiments, the URD does not strictly need to be derived from an E3 ubiquitin ligase, and may otherwise be derived from other sources, such as viral analogues of E3 ubiquitin ligases that have ubiquitin complex recruitment function (for example, the VIF protein of HIV). The URD may be derived from any of the diverse family of E3 ubiquitin ligases based on factors such as size, localization in the cell (e.g., cytoplasm and/or nucleus), and orientation of the URD domain within the E3 ubiquitin ligase (i.e., if the URD appears N-terminally or C-terminally to the substrate recognition domain). In some embodiments, exemplary URD domains may include, but are not limited to, U-box, RING, HECT, F-box (Cull-SKP1), BTB (Cul3), and H-box (Cul4-DDB1) domains. In some embodiments, exemplary URDs may be derived from E3 ubiquitin ligases including but not limited to CHIP (also termed STUB1), RNF114, RNF125, RNF138, RNF166, NHLRC1, CBL-b (Y363E), TRIM21, E6AP, FBXW7 alpha, FBXW7 beta, beta-TRCP, Keap1, SPOP, or DCAF1. It is envisioned that any truncation or fragment of these E3 ubiquitin ligases that retain ubiquitin-proteasome recruiting activity may be used as a URD in the embodiments herein. In some embodiments, the URD is derived from a RING domain (e.g., RNF114, RNF125, RNF138, RNF166, NHLRC1, CBL-b (Y363E)) or an H-box (Cul4-DDB1) domain (e.g., DCAF1), or any truncation or fragment thereof that retains ubiquitin-proteasome recruiting activity.

The ubiquitin-proteasome system recruiting domain (URD) should be understood as a protein, or fragment or truncation thereof, that can function to recruit a ubiquitination complex (e.g., can recruit an E2 ubiquitin conjugating enzyme). In some embodiments, the URD is derived from an E3 ubiquitin ligase. In some embodiments, the URD is derived from CHIP, RNF114, RNF125, RNF138, RNF166, NHLRC1, CBL-b (Y363E), TRIM21, E6AP, FBXW7 alpha, FBXW7 beta, beta-TRCP, Keap1, SPOP, or DCAF1, or a truncation or fragment thereof that retains ubiquitin-proteasome recruiting activity. In some embodiments, the URD is derived from CHIP, RNF114, RNF125 (e.g., RNF125.2), RNF138, RNF166, NHLRC1, CBL-b (Y363E), TRIM21, E6AP, FBXW7 alpha, FBXW7 beta, beta-TRCP, Keap1, SPOP (e.g., SPOP.2 or SPOP.3), or DCAF1, or a truncation or fragment thereof that retains ubiquitin-proteasome recruiting activity. In some embodiments, the URD is selected from a RING domain and an H-box (Cul4-DDB1) domain, or a truncation or fragment thereof that retains ubiquitin-proteasome recruiting activity. In some embodiments, the RING domain is RNF114, RNF125, RNF138, RNF166, NHLRC1, CBL-b (Y363E), or TRIM21. In some embodiments, the H-box domain is DCAF1. In some embodiments, the URD is selected from: the URD derived from a protein which localizes to the cytoplasm; the URD derived from a protein which localizes to the nucleus; and the URD derived from a protein which localizes to the cytoplasm and the nucleus; or a truncation or fragment of the URD that retains ubiquitin-proteasome recruiting activity. In some embodiments, the protein which localizes to the cytoplasm is FBXW7 beta. In some embodiments, the protein which localizes to the nucleus is selected from FBXW7 alpha and SPOP. In some embodiments, the protein which localizes to the cytoplasm and the nucleic is selected from CHIP, RNF114, RNF125, RNF138, RNF166, NHLRC1, CBL-b (Y363E), TRIM21, E6AP, and DCAF1. In some embodiments, the URD is derived from an E3 ligase selected from the group consisting of NHLRC1, DCAF1, RNF125, RNF165, RNF4, and SPOP. In some embodiments, the URD is derived from an E3 ligase selected from the group consisting of optionally NHLRC1, RNF125, RNF165, RNF4 and SPOP. In some embodiments, the URD is derived from an E3 ligase selected from the group consisting of optionally NHLRC1, RNF125, RNF165, and RNF4. In some embodiments, the URD is selected from the group consisting of NHLRC1.1, DCAF1.1, RNF125.2, RNF125.3, RNF125.4, RNF125.5, RNF125.6, RNF165.1, RNF4.1, SPOP.2, and SPOP.3. In some embodiments, the URD is selected from the group consisting of NHLRC1.1, RNF125.2, RNF125.3, RNF125.4, RNF125.5, RNF125.6, RNF165.1, RNF4.1, SPOP.2, and SPOP.3. In some embodiments, the URD is selected from the group consisting of NHLRC1.1, RNF125.2, RNF165.1, and RNF4.1. In some embodiments, the protein is an E3 ubiquitin ligase. In some embodiments, the URD is, is about, is at least, is at least about, is not more than, or is not more than about, 370, 369, 368, 367, 366, 365, 364, 363, 362, 361, 360, 300, 250, 200, 175, 170, 165, 160, 155, 150, 145, 140, 135, 130, 129, 128, 127, 126, 125, 124, 123, 120, 115, 110, 105, 100, 95, 90, 85, 80, 75, 70, 65, 60, 55, 50, 47, 46, 45, 44, 43, 42, 41, 40, 39, 38, 37, 36, 35, 34, 33, 32, 31, 30, 25, or 20 amino acids long, or a range defined by any two of the preceding values, for example, 370-20, 370-120, 120-20, 80-20, or 360-150 amino acids long. In some embodiments, the URD comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NO: 29-43, or a truncation or fragment thereof that retains ubiquitin-proteasome recruiting activity. In some embodiments, the URD comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NO: 1836-1895, or a truncation or fragment thereof that retains ubiquitin-proteasome recruiting activity. In some embodiments, the URD comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NO: 1855, 1856, 1858, 1870-1874, 1880, and 1886, or a truncation or fragment thereof that retains ubiquitin-proteasome recruiting activity. In some embodiments, the URD comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NO: 1858, 1870, 1880, and 1886, or a truncation or fragment thereof that retains ubiquitin-proteasome recruiting activity. In some embodiments where the amino acid sequence differs from a recited reference sequence, the difference is the result of the inclusion of one or more modifications relative to the reference sequence. In some embodiments, the one or more modifications comprise a substitution, insertion and/or deletion. In some embodiments, the substitution is a conservative substitution. In some embodiments, the % sequence identity is calculated over the entirety of the reference sequence.

In some embodiments, the URD is selected from: a URD derived from a protein which localizes to the cytoplasm, optionally selected from FBXW7 beta and Keap1; a URD derived from a protein which localizes to the nucleus, optionally selected from FBXW7 alpha, RNF165, and SPOP; and a URD derived from a protein which localizes to the cytoplasm and the nucleus, optionally selected from beta-TRCP, CHIP, RNF114, RNF125, RNF138, RNF166, NHLRC1, CBL-b (Y363E), TRIM21, E6AP, DCAF1 VIF, RNF11, RNF111, RNF115, RNF12, RNF128, RNF149, RNF152, RNF165, RNF182, RNF20, RNF25, RNF4, RNF6, TRIM32, ZNRF1, ZNRF4.

Table 2 depicts various E3 ubiquitin ligases and respective URDs embodied herein. In some embodiments of the polypeptide comprising or consisting of a portion derived from a protein, the portion comprises an exemplary URD described below and/or in Table 2. In some embodiments of the dual-specific bifunctional polypeptide, the dual-specific bifunctional polypeptide comprises an exemplary URD described below and/or in Table 2.

One exemplary URD embodied herein is the CHIP URD (CHIP.1) (SEQ ID NO: 29, 1836), which is amino acids 128-303 of the full-length CHIP protein (SEQ ID NO: 14; Uniprot #Q9UNE7; where CHIP is also referred to as STUB1). It is envisioned that alternative portions, fragments, or truncations of the CHIP protein having ubiquitin-proteasome recruiting activity may also be used.

An additional exemplary URD embodied herein is the DCAF1 URD (DCAF1.1) (SEQ ID NO: 43, 1837), which is amino acids 1045-1079 of the full length DCAF1 protein (SEQ ID NO: 28; Uniprot #Q9Y4B6). It is envisioned that alternative portions, fragments, or truncations of the DCAF1 protein having ubiquitin-proteasome recruiting activity may also be used.

An additional exemplary URD embodied herein is the E6AP URD (E6AP.1) (SEQ ID NO: 37, 1838), which is amino acids 520-875 of the full length E6AP protein (SEQ ID NO: 22; Uniprot #Q05086). It is envisioned that alternative portions, fragments, or truncations of the E6AP protein having ubiquitin-proteasome recruiting activity may also be used.

An additional exemplary URD embodied herein is the beta-TRCP URD (bTRCP.1) (SEQ ID NO: 40, 1839), which is amino acids 2-287 of the full length beta-TRCP protein (SEQ ID NO: 25; Uniprot #Q3ULA). It is envisioned that alternative portions, fragments, or truncations of the beta-TRCP protein having ubiquitin-proteasome recruiting activity may also be used.

An additional exemplary URD embodied herein is the FBXW7 alpha URD (FBXW7a.1) (SEQ ID NO: 38, 1840), which is amino acids 2-368 of the full length FBXW7 alpha protein (SEQ ID NO: 23; Uniprot #Q969H0). Additional URDs derived from FBXW7 alpha are presented in Table 2 (SEQ ID NOs: 1841-1848). It is envisioned that alternative portions, fragments, or truncations of the FBXW7 alpha protein having ubiquitin-proteasome recruiting activity may also be used.

An additional exemplary URD embodied herein is the FBXW7 beta URD (FBXW7b.1) (SEQ ID NO: 39, 1849), which is amino acids 2-228 of the full length FBXW7 beta protein (SEQ ID NO: 24; Uniprot #Q969H0). Additional URDs derived from FBXW7 beta are presented in Table 2 (SEQ ID NOs: 1850-1852). It is envisioned that alternative portions, fragments, or truncations of the FBXW7 beta protein having ubiquitin-proteasome recruiting activity may also be used.

An additional exemplary URD embodied herein is the Keap1 URD (Keap1.1) (SEQ ID NO: 41, 1853)), which is amino acids 2-322 of the full length Keap1 protein (SEQ ID NO: 26; Uniprot #Q14145). It is envisioned that alternative portions, fragments, or truncations of the Keap1 protein having ubiquitin-proteasome recruiting activity may also be used.

An additional exemplary URD embodied herein is the SPOP URD (SPOP.1) (SEQ ID NO: 42, 1854), which is amino acids 147-374 of the full length SPOP protein (SEQ ID NO: 27; Uniprot #043791). Additional URDs derived from SPOP are presented in Table 2 (SEQ ID NOs: 1855-1856). It is envisioned that alternative portions, fragments, or truncations of the SPOP protein having ubiquitin-proteasome recruiting activity may also be used.

An additional exemplary URD embodied herein is the VIF URD (VIF.1) (SEQ ID NO: 1857)), which is amino acids 116-157 of the full length VIF HV-1 protein (SEQ ID NO: 2167; Uniprot #P12504). It is envisioned that alternative portions, fragments, or truncations of the VIF protein having ubiquitin-proteasome recruiting activity may also be used.

An additional exemplary URD embodied herein is the NHLRC1 URD (NHLRC1.1) (SEQ ID NO: 34, 1858), which is amino acids 7-98 of the full length NHLRC1 protein (SEQ ID NO: 19; Uniprot #Q6VVB1). Additional URDs derived from NHLRC1 are presented in Table 2 (SEQ ID NOs: 1859-1862). It is envisioned that alternative portions, fragments, or truncations of the NHLRC1 protein having ubiquitin-proteasome recruiting activity may also be used.

An additional exemplary URD embodied herein is the RNF11 URD (RNF11.1) (SEQ ID NO: 1863), which is amino acids 97-150 of the full length RNF11 protein (SEQ ID NO: 2156; Uniprot #Q9Y3C5). It is envisioned that alternative portions, fragments, or truncations of the RNF11 protein having ubiquitin-proteasome recruiting activity may also be used.

An additional exemplary URD embodied herein is the RNF111 URD (RNF111.1) (SEQ ID NO: 1864), which is amino acids 934-994 of the full length RNF111 protein (SEQ ID NO: 2159; Uniprot #Q6ZNA4). It is envisioned that alternative portions, fragments, or truncations of the RNF111 protein having ubiquitin-proteasome recruiting activity may also be used.

An additional exemplary URD embodied herein is the RNF114 URD (RNF114.1) (SEQ ID NO: 30, 1865), which is amino acids 25-119 of the full length RNF114 protein (SEQ ID NO: 15; Uniprot #Q9Y508). It is envisioned that alternative portions, fragments, or truncations of the RNF114 protein having ubiquitin-proteasome recruiting activity may also be used.

An additional exemplary URD embodied herein is the RNF115 URD (RNF115.1) (SEQ ID NO: 1866), which is amino acids 226-270 of the full length RNF115 protein (SEQ ID NO: 2160; Uniprot #Q9Y4L5). It is envisioned that alternative portions, fragments, or truncations of the RNF115 protein having ubiquitin-proteasome recruiting activity may also be used.

Additional exemplary URDs embodied herein are the RNF12 URDs (RNF12.1-.2) (SEQ ID NOs: 1867-1868), which are amino acids 547-615 and 547-624 of the full length RNF12 protein (SEQ ID NO: 2156; Uniprot #Q9NVW2). It is envisioned that alternative portions, fragments, or truncations of the RNF12 protein having ubiquitin-proteasome recruiting activity may also be used.

An additional exemplary URD embodied herein is the RNF125 URD (RNF125.1) (SEQ ID NO: 31, 1869), which is amino acids 2-128 of the full length RNF125 protein (SEQ ID NO: 16; Uniprot #Q96EQ8). Additional URDs derived from RNF125 are presented in Table 2 (SEQ ID NOs: 1870-1875). It is envisioned that alternative portions, fragments, or truncations of the RNF125 protein having ubiquitin-proteasome recruiting activity may also be used.

An additional exemplary URD embodied herein is the RNF128 URD (RNF128.1) (SEQ ID NO: 1876), which is amino acids 274-326 of the full length RNF128 protein (SEQ ID NO: 2161; Uniprot #Q8TEB7). It is envisioned that alternative portions, fragments, or truncations of the RNF128 protein having ubiquitin-proteasome recruiting activity may also be used.

An additional exemplary URD embodied herein is the RNF138 URD (RNF138.1) (SEQ ID NO: 32, 1877)), which is amino acids 14-113 of the full length RNF138 protein (SEQ ID NO: 17; Uniprot #Q8WVD3). It is envisioned that alternative portions, fragments, or truncations of the RNF138 protein having ubiquitin-proteasome recruiting activity may also be used.

An additional exemplary URD embodied herein is the RNF149 URD (RNF149.1) (SEQ ID NO: 1878), which is amino acids 267-311 of the full length RNF149 protein (SEQ ID NO: 2162; Uniprot #Q8NC42). It is envisioned that alternative portions, fragments, or truncations of the RNF149 protein having ubiquitin-proteasome recruiting activity may also be used.

An additional exemplary URD embodied herein is the RNF152 URD (RNF152.1) (SEQ ID NO: 1879), which is amino acids 9-55 of the full length RNF152 protein (SEQ ID NO: 2163; Uniprot #Q8N8N0). It is envisioned that alternative portions, fragments, or truncations of the RNF152 protein having ubiquitin-proteasome recruiting activity may also be used.

An additional exemplary URD embodied herein is the RNF165 URD (RNF165.1) (SEQ ID NO: 1880), which is amino acids 259-339 of the full length RNF165 protein (SEQ ID NO: 2164; Uniprot #Q6ZSG1). It is envisioned that alternative portions, fragments, or truncations of the RNF165 protein having ubiquitin-proteasome recruiting activity may also be used.

An additional exemplary URD embodied herein is the RNF166 URD (RNF166.1) (SEQ ID NO: 33, 1881), which is amino acids 26-126 of the full length RNF166 protein (SEQ ID NO: 18; Uniprot #Q96A37). It is envisioned that alternative portions, fragments, or truncations of the RNF166 protein having ubiquitin-proteasome recruiting activity may also be used.

Additional exemplary URDs embodied herein are the RNF182 URDs (RNF182.1-.2) (SEQ ID NOs: 1882-1883), which are amino acids 19-74 and 17-92 of the full length RNF182 protein (SEQ ID NO: 2165; Uniprot #Q8N6D2). It is envisioned that alternative portions, fragments, or truncations of the RNF182 protein having ubiquitin-proteasome recruiting activity may also be used.

An additional exemplary URD embodied herein is the RNF20 URD (RNF20.1) (SEQ ID NO: 1884), which is amino acids 906-975 of the full length RNF20 protein (SEQ ID NO: 2157; Uniprot #Q5VTR2). It is envisioned that alternative portions, fragments, or truncations of the RNF20 protein having ubiquitin-proteasome recruiting activity may also be used.

An additional exemplary URD embodied herein is the RNF25 URD (RNF25.1) (SEQ ID NO: 1885), which is amino acids 129-254 of the full length RNF25 protein (SEQ ID NO: 2158; Uniprot #Q96BH1). It is envisioned that alternative portions, fragments, or truncations of the RNF25 protein having ubiquitin-proteasome recruiting activity may also be used.

An additional exemplary URD embodied herein is the RNF4 URD (RNF4.1) (SEQ ID NO: 1886), which is amino acids 130-190 of the full length RNF4 protein (SEQ ID NO: 2153; Uniprot #P78317). It is envisioned that alternative portions, fragments, or truncations of the RNF4 protein having ubiquitin-proteasome recruiting activity may also be used.

Additional exemplary URDs embodied herein are the RNF6 URDs (RNF6.1-2) (SEQ ID NOs: 1887-1888), which are amino acids 609-677 and 609-685 of the full length RNF6 protein (SEQ ID NO: 2154; Uniprot #Q9Y252). It is envisioned that alternative portions, fragments, or truncations of the RNF6 protein having ubiquitin-proteasome recruiting activity may also be used.

An additional exemplary URD embodied herein is the TRIM21 URD (SEQ ID NO: 36), which is amino acids 1-277 of the full length TRIM21 protein (SEQ ID NO: 21; Uniprot #P19474). Additional URDs derived from TRIM21 are presented in Table 2 (SEQ ID NOs: 1889-1891). It is envisioned that alternative portions, fragments, or truncations of the TRIM21 protein having ubiquitin-proteasome recruiting activity may also be used.

An additional exemplary URD embodied herein is the TRIM32 URD (TRIM32.1) (SEQ ID NO: 1892), which is amino acids 7-96 of the full length TRIM32 protein (SEQ ID NO: 2166; Uniprot #Q13049). It is envisioned that alternative portions, fragments, or truncations of the TRIM32 protein having ubiquitin-proteasome recruiting activity may also be used.

An additional exemplary URD embodied herein is the ZNRF1 URD (ZNRF1.1) (SEQ ID NO: 1893), which is amino acids 142-227 of the full length ZNRF1 protein (SEQ ID NO: 2168; Uniprot #Q8ND25). It is envisioned that alternative portions, fragments, or truncations of the ZNRF1 protein having ubiquitin-proteasome recruiting activity may also be used.

An additional exemplary URD embodied herein is the ZNRF4 URD (ZNRF4.1) (SEQ ID NO: 1894), which is amino acids 307-354 of the full length ZNRF4 protein (SEQ ID NO: 2169; Uniprot #Q8WWF5). It is envisioned that alternative portions, fragments, or truncations of the ZNRF4 protein having ubiquitin-proteasome recruiting activity may also be used.

An additional exemplary URD embodied herein is the CBL-b URD (CBLb.1) (SEQ ID NO: 35, 1895), which is amino acids 349-427 of the full length CBL-b protein (SEQ ID NO: 20; Uniprot #Q13191). It is noted that the CBL-b sequence provided herein harbors a Y363E mutation, which confers constitutive ubiquitin ligase activity. In some embodiments, the Y363E mutation is not present. It is envisioned that alternative portions, fragments, or truncations of the CBL-b protein having ubiquitin-proteasome recruiting activity may also be used.

Additional URDs are disclosed in PCT/US2023/066619, filed May 4, 2023, which is herein incorporated by reference in its entirety.

### Localization Sequences

Localization of degraders to the correct subcellular compartment may be important for the ability to degrade a target protein, e.g., if the target is localized to the nucleus, or if degradation of target protein only in the nucleus but not cytoplasm is desired. In some embodiments provided herein are polypeptides, or dual-specific bifunctional polypeptides, which are engineered (e.g., by modification of an endogenous localization peptide sequence, by adding localization peptide sequences, and/or by selecting a component which has an endogenous localization sequence) to direct the polypeptide or dual-specific bifunctional polypeptide to a particular subcellular compartment, e.g., the cytoplasm, the nucleus, or both.

In some embodiments, the polypeptide comprising a portion derived from an E3 ligase or viral homolog thereof comprises one or more localization peptide sequences. In some embodiments, the dual-specific bifunctional polypeptide comprises one or more localization peptide sequences. In some embodiments, the polypeptide comprising a portion, or the dual-specific bifunctional polypeptide comprises one or more localization peptide sequences that direct the polypeptide or dual-specific bifunctional polypeptide to a subcellular compartment or compartments, optionally wherein the subcellular compartment is the nucleus and/or the cytoplasm. In some embodiments, the one or more localization peptide sequences direct the polypeptide or dual-specific bifunctional polypeptide to a desired subcellular compartment or compartments, optionally wherein the one or more localization peptide sequences are selected or designed to direct the polypeptide or dual-specific bifunctional polypeptide to a desired subcellular compartment or compartments. In some embodiments, the one or more localization peptide sequences comprise or consist of a nuclear localization signal (NLS) peptide and/or a nuclear export signal (NES) peptide. In some embodiments, the dual-specific bifunctional polypeptide further comprises a nuclear localization signal (NLS) peptide. In some embodiments, the NLS peptide is located at the N-terminus of the dual-specific bifunctional polypeptide, the C-terminus of the dual-specific bifunctional polypeptide, or internally within the dual-specific bifunctional polypeptide. In some embodiments, the NLS signal is an NLS that naturally occurs in one or more of the components of the dual-specific bifunctional polypeptide, such as an NLS that is naturally found in the first and/or second targeting moiety and/or URD. For example, the first and/or second targeting moiety and/or URD of a dual-specific bifunctional polypeptide may comprise an NLS within their sequence. In some embodiments the NLS is an NLS that is engineered into the dual-specific bifunctional polypeptide, for example, added N-terminally, C-terminally, or internally within a component of the dual-specific bifunctional polypeptide, such as the first and/or second targeting moiety and/or URD. In some embodiments, the NLS peptide comprises or consists of the amino acid sequence of SEQ ID NO: 51. However, other suitable NLS peptides generally known in the art may also be used. In some embodiments the localization sequence is a sequence from Table 3 or 4.

In some embodiments, the one or more localization peptide sequences comprise or consist of an endogenous localization peptide sequence of the first and/or second targeting moiety and/or URD. In some embodiments, the one or more localization peptide sequences comprise or consist of an endogenous localization peptide sequence of the URD, for example the localization sequences disclosed in Table 4. In some embodiments, the URD is selected from: a URD derived from a protein which localizes to the cytoplasm, optionally selected from FBXW7 beta and Keap1; a URD derived from a protein which localizes to the nucleus, optionally selected from FBXW7 alpha, RNF165, and SPOP; and a URD derived from a protein which localizes to the cytoplasm and the nucleus, optionally selected from beta-TRCP, CHIP, RNF114, RNF125, RNF138, RNF166, NHLRC1, CBL-b (Y363E), TRIM21, E6AP, DCAF1 VIF, RNF11, RNF111, RNF115, RNF12, RNF128, RNF149, RNF152, RNF165, RNF182, RNF20, RNF25, RNF4, RNF6, TRIM32, ZNRF1, ZNRF4.

In some embodiments, the one or more localization peptide sequences comprise or consist of an endogenous localization peptide sequence of the URD and/or first and/or second targeting moiety that is modified, wherein the modification alters the subcellular compartment or compartments to which the dual-specific bifunctional polypeptide is directed as compared to the native endogenous localization peptide sequence. In some embodiments, the modification comprises or consists of a substitution, truncation or deletion of an endogenous localization peptide sequence. In some embodiments:
b. the native endogenous localization peptide sequence directs the polypeptide to the nucleus, and the modified endogenous localization peptide sequence directs the polypeptide to the cytoplasm;
c. the native endogenous localization peptide sequence directs the polypeptide to the cytoplasm, and the modified endogenous localization peptide sequence directs the polypeptide to the nucleus;
d. the native endogenous localization peptide sequence directs the polypeptide to the nucleus and the cytoplasm, and the modified endogenous localization peptide sequence directs the polypeptide to only the cytoplasm;
e. the native endogenous localization peptide sequence directs the polypeptide to the nucleus and the cytoplasm, and the modified endogenous localization peptide sequence directs the polypeptide to only the nucleus;
f. the native endogenous localization peptide sequence directs the polypeptide to the nucleus, and the modified endogenous localization peptide sequence directs the polypeptide to the nucleus and the cytoplasm; or
g. the native endogenous localization peptide sequence directs the polypeptide to the cytoplasm, and the modified endogenous localization peptide sequence directs the polypeptide to the nucleus and the cytoplasm.

In some embodiments, the one or more localization peptide sequences comprises or consists of an endogenous localization peptide sequence of the URD that is modified, wherein the native endogenous localization peptide sequence directs the polypeptide to the nucleus, and the modified endogenous localization peptide sequence directs the polypeptide to the cytoplasm. In some embodiments, the modified endogenous localization peptide sequence comprises or consists of a modified SPOP URD endogenous localization peptide sequence, optionally wherein the URD is SPOP.3.

In some embodiments, the one or more localization peptide sequences comprise or consist of an exogenous localization peptide sequence. In some embodiments, the one or more localization peptide sequences comprise or consist of an NLS peptide that is or is a derivative of the NLS of MYC, simian virus 40 (SV40), SPOP, nucleoplasmin, 53BP1, Hrp1. In some embodiments, the one or more localization peptide sequences comprise or consists of an NLS sequence disclosed in Table 3. In some embodiments, the one or more localization peptide sequences comprise or consist of an NLS comprising or consisting of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NOs: 1896-1909, or SEQ ID NOs: 1869-1904. In some embodiments where the amino acid sequence differs from a recited reference sequence, the difference is the result of the inclusion of one or more modifications relative to the reference sequence. In some embodiments, the one or more modifications comprise a substitution, insertion and/or deletion. In some embodiments, the substitution is a conservative substitution. In some embodiments, the % sequence identity is calculated over the entirety of the reference sequence.

In some embodiments, the polypeptide comprising a portion derived from an E3 ligase or viral homolog thereof comprises 2, 3, 4, or more localization peptide sequences, optionally wherein the polypeptide comprises 2, 3, 4 or more copies of the same localization sequence in series. In some embodiments, the dual-specific bifunctional polypeptide comprises 2, 3, 4, or more localization peptide sequences, optionally wherein the dual-specific bifunctional polypeptide comprises 2, 3, 4 or more copies of the same localization sequence in series. In some embodiments, the polypeptide or the dual-specific bifunctional polypeptide comprises one or more localization peptide sequences located at a location selected from the group consisting of: the N-terminus of the dual-specific bifunctional polypeptide; the C-terminus of the dual-specific bifunctional polypeptide; internally within the dual-specific bifunctional polypeptide; as part of the URD, optionally an endogenous portion of the URD; as a part of the first and/or second targeting moiety, optionally an endogenous portion of the URD; and a combination of any of the foregoing. In some embodiments, a first localization peptide sequence is located at the N-terminus of the polypeptide or the dual-specific bifunctional polypeptide and a second localization peptide sequence is located at the C-terminus of the polypeptide or the dual-specific bifunctional polypeptide.

TABLE 3
Exemplary NLS sequences

| | | | Uniprot ID of |
|---|---|---|---|
| SEQ ID NO | Name | Parental Protein | Parental Protein |
| 1896 | spNLS.2 | SPOP | O43791 |
| 1897 | spNLS | SPOP | O43791 |
| 1898 | brpNLS | Hrp1 | Q99383 |
| 1899 | bpNLS | 53BP1 | Q12888 |
| 1900 | nlpNLS | Nucleoplasmin | P05221 |
| 1901 | 3x(svNLS) | Large T antigen (SV40) | P03070 |
| 1902 | 3x(mycNLS) | MYC | P01106 |
| 1903 | svNLS | Large T antigen (SV40) | P03070 |
| 1904 | mycNLS | MYC | P01106 |

TABLE 4
Exemplary URD endogenous NLS/NES sequences

| SEQ | | NLS | | | Uniprot ID |
|---|---|---|---|---|---|
| ID | | or | | Parental | of Parental |
| NO | Name | NES | E3 Family | protein | Protein |
| 1905 | CHIP.NLS | NLS | U-box | CHIP | Q9UNE7 |
| 1906 | SPOP.NLS | NLS | BTB/Cul3 | SPOP | O43791 |
| 1907 | RNF165.NLS | NLS | RING | RNF165 | Q6ZSG1 |
| 1908 | FBXW7a.NLS | NLS | F-box/Cul1 | FBXW7a | Q969H0 |
| 1909 | NHLRC1.NLS | NLS | RING | NHLRC1 | Q6VVB1 |
| 1910 | NHLRC1.NES | NES | RING | NHLRC1 | Q6VVB1 |
| 1911 | E6AP.NES | NES | HECT | E6AP | Q05086 |
| 1912 | Keap1.NES | NES | BTB/Cul3 | Keap1 | Q14145 |
| 1913 | RNF12.NES | NES | RING | RNF12 | Q9NVW2 |
| 1914 | RNF138.NES | NES | RING | RNF138 | Q8WVD3 |
| 1915 | RNF166.NES | NES | RING | RNF166 | Q96A37 |
| 1916 | TRIM21.NES | NES | RING | TRIM21 | P19474 |
| 1917 | TRIM32.NES | NES | RING | TRIM32 | Q13049 |

In some embodiments, the NLS peptide comprises or consists of the amino acid sequence of SEQ ID NOs: 51 or 1897-1903. In some embodiments, the NLS peptide is derived from MYC NLS, SV40 NLS, 3XMYC NLS, 3xSV40 NLS, SPOP NLS, nucleoplasmin NLS, 53BP1 NLS, or Hrp1 NLS (SEQ ID NOs: 51, 1897-1903. In some embodiments, other suitable NLS peptides generally known in the art are contemplated. Some URDSs derived from E3 ubiquitin ligases contain an endogenous NLS, for example, SPOP and SPOP.2. In some embodiments, the dual-specific bifunctional polypeptide contains a URD with an endogenous NLS. In some embodiments, the dual-specific bifunctional polypeptide contains a URD with an endogenous NLS and an exogenous NLS. In some embodiments, the dual-specific bifunctional polypeptide contains a URD with a truncated or deleted endogenous NLS (e.g., SPOP.3) and an exogenous NLS.

Additional localization sequences are disclosed in PCT/US2023/066619, filed May 4, 2023, which is herein incorporated by reference in its entirety.

### Dual-Specific Bifunctional Polypeptides: General Characteristics

The following characteristics may apply to embodiments of the dual-specific bifunctional polypeptides disclosed herein. In some embodiments, the dual-specific bifunctional polypeptide comprises a first and second targeting moiety (also referred to as a target “binder” or “binding moiety”) that is capable of binding to the target protein(s). In some embodiments, first and second target binding moieties are capable of binding the same target protein, optionally at different portions of the target protein. In some embodiments, combination of any two of the targeting moieties and a URD disclosed herein are contemplated to be used in the construction of the dual-specific bifunctional polypeptide. In some embodiments, the first and second targeting moieties are two copies of the same targeting moiety (e.g., both are Omomyc). In some embodiments, the first and second targeting moieties target the same protein (e.g., CTNNB1), but are not the same targeting moieties (e.g., TCF4.6 and TCF4.16). In some embodiments, the first targeting moiety and the second targeting moiety target different target proteins. In some embodiments the first targeting moiety and the second targeting moiety target two proteins selected from the combinations of MYC/CTNNB1, MYC/PCNA and CTNNB1/PCNA. In some embodiments, proximity of the dual-specific bifunctional polypeptide to the first and/or the second target protein through binding of the targeting moiety induces ubiquitination of the first and/or the second target protein via the URD, thereby promoting proteasome-mediated degradation of the target protein(s). In some embodiments, the first and the second target proteins can be or are bound by the dual-specific bifunctional polypeptide simultaneously, in some embodiments only one of the first and second target proteins can be or are bound by the dual-specific bifunctional polypeptide at a time. In some embodiments, the dual-specific bifunctional polypeptide may also further comprise additional components. In some embodiment the first and/or second targeting moiety binds selectively or specifically to the target protein(s). In some embodiments, the first and/or second targeting moiety is, or is less than, 260, 259, 258, 257, 256, 255, 254, 253, 252, 251, 250, 245, 240, 235, 230, 225, 220, 215, 210, 205, 200, 195, 190, 185, 180, 175, 170, 165, 160, 155, 150, 145, 140, 135, 130, 129, 128, 127, 126, 125, 124, 123, 122, 121, 120, 119, 118, 117, 116, 115, 114, 113, 112, 110, 109, 108, 107, 106, 105, 104, 103, 102, 101, 100, 99, 98, 97, 96, 95, 94, 93, 92, 91, 90, 89, 88, 87, 86, 85, 84, 83, 82, 81, 80, 75, 70, 65, 60, 55, 54, 53, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 amino acids long, or a range defined by any two of the preceding values, optionally 10-260, 10-200, 10-130, 25-260, 25-200, 25-130, 35-260, 35-200, 35-130, 50-260, 50-200, or 50-130 amino acids long. In some embodiments, the first and/or second targeting moiety comprises or consists of an endogenous binding partner of the target protein, an antibody, Fab, F(ab′)₂, Fab′, scFv, single domain antibody (sdAb), V_{H} domain, V_{L} domain, V_{H}H, V_{NAR}, diabody, intrabody, DARPin, monobody, affibody, avimer, or any binding fragment or derivative thereof.

In some embodiments, the first and/or second targeting moiety comprises or consists of an endogenous protein binding partner of the target protein, a fragment and/or a derivative thereof. In some embodiments, the first and/or second targeting moiety is, or is less than, 100, 99, 98, 97, 96, 95, 94, 93, 92, 91, 90, 89, 88, 87, 86, 85, 84, 83, 82, 81, 80, 75, 70, 65, 60, 55, 54, 53, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 amino acids long, or a range defined by any two of the preceding values. In some embodiments, the first and/or second targeting moiety is 10-100, 10-25, 14-100, 14-25, 30-100, 30-50, 30-45, or 80-100 amino acids long.

In some embodiments, the first and/or second targeting moiety comprises or consists of a sdAb. In some embodiments, the first and/or second targeting moiety comprises or consists of either a V_{H} domain or a V_{L} domain of an IgG antibody. In some embodiments, the first and/or second targeting moiety comprises or consists of a V_{H}H, V_{H}, or V_{NAR} domain of a heavy chain antibody (HcAb). In some embodiments, the first and/or second targeting moiety comprises or consists of a V_{H}H or V_{L} domain. In some embodiments, the first and/or second targeting moiety comprises or consists of a sdAb and the first and/or second targeting moiety is, or is less than, 170, 165, 160, 155, 150, 145, 140, 135, 130, 129, 128, 127, 126, 125, 124, 123, 122, 121, 120, 119, 118, 117, 116, 115, 114, 113, 112, 110, 109, 108, 107, 106, 105, 104, 103, 102, 101, 100, 99, 98, 97, 96, 95, 94, 93, 92, 91, or 90, amino acids long, or a range defined by any two of the preceding values, optionally 90-150, 90-130, 110-150, 110-130, 110-125, or 114-124 amino acids long. In some embodiments, the first and/or second targeting moiety comprises or consists of an scFv. In some embodiments, the first and/or second targeting moiety comprises or consists of an scFv first and/or second targeting moiety is, or is less than, 260, 259, 258, 257, 256, 255, 254, 253, 252, 251, 250, 245, 240, 235, 230, 225, 220, 215, 210, 205, 200, 195, or 190 amino acids long, or a range defined by any two of the preceding values, optionally 190-260, 200-260, or 230-260 amino acids long.

In some embodiments, the first and/or second targeting moieties and/or dual-specific bifunctional polypeptide of the present disclosure have an equilibrium dissociation constant of less (that is superior binding) than about 10⁻⁷ or 10⁻⁸ M, for example, less than about 10⁻⁹ M or 10⁻¹⁰ M, in some embodiments, less than about 10⁻¹¹ M, 10⁻¹² M, or 10⁻¹³ M. In some embodiments, the first and/or second targeting moiety binds selectively to the target protein(s). In some embodiments, the first and/or second targeting moiety binds specifically to the target protein(s).

In some embodiments, the first and/or second targeting moiety is generally any protein that is able to bind to the target protein(s) with specificity. In some embodiments, the first and/or second targeting moiety is an endogenous binding partner of the target protein, antibody, Fab, F(ab′)₂, Fab′, scFv, sdAb, V_{H} domain, V_{L} domain, V_{H}H, V_{NAR}, diabody, intrabody, DARPin, monobody, affibody, avimer, or any fragment or derivative thereof, or any other suitable targeting moiety generally known in the art. In some embodiments, the first and/or second targeting moiety is, is about, is at least, is at least about, is not more than, or is not more than about, 260, 259, 258, 257, 256, 255, 254, 253, 252, 251, 250, 245, 240, 235, 230, 225, 220, 215, 210, 205, 200, 195, 190, 185, 180, 175, 170, 165, 160, 155, 150, 145, 140, 135, 130, 129, 128, 127, 126, 125, 124, 123, 122, 121, 120, 119, 118, 117, 116, 115, 114, 113, 112, 110, 109, 108, 107, 106, 105, 104, 103, 102, 101, 100, 99, 98, 97, 96, 95, 94, 93, 92, 91, 90, 89, 88, 87, 86, 85, 84, 83, 82, 81, 80, 75, 70, 65, 60, 55, 54, 53, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 amino acids long, or a range defined by any two of the preceding values, for example, 260-10, 260-120, 120-10, 60-10, or 250-50 amino acids long.

### Exemplary Targets of Dual-Specific Bifunctional Polypeptides

In some embodiments, the target protein is generally a protein that is involved in disease pathology, for example, cancer. In some embodiments, the disease or pathology is a neurodegenerative disease. In some embodiments, the dual-specific bifunctional polypeptide comprises a first and second targeting moiety that is capable of binding (optionally selectively or specifically) to target protein selected from MYC, (optionally c-MYC), beta catenin 1 (CTNNB1), and proliferating cell nuclear antigen (PCNA). In some embodiments, the dual-specific bifunctional polypeptide targets a protein selected from MYC, (optionally c-MYC), and CTNNB1. In some embodiments, the target protein is a wild-type and/or a mutant form of the target protein, optionally wherein the targeting moiety preferentially or selectively binds the mutant form of the target protein relative to the wild-type form of the target protein. In some embodiments, the mutant form of the target protein is CTNNB1 (SEQ ID NO: 3) comprising one or more mutations selected from: D32Y; D32N; D32V; D32G; S33Y; S33L; S33F; S33C; S33P; G34V; G34E; I35S; H36P; S37C; S37P; S37P and D207G; T41A; T41I; T42_K49del; S45F and Y670X wherein X indicates termination; $45C; S45F; S45P; S45Y; and S4Sdel.

In some embodiments, the first and/or second targeting moiety binds, or binds at least in part, to at least a portion of: a) the DNA binding domain of MYC; or b) the armadillo domain of CTNNB1. In some embodiments, the first and/or second targeting moiety binds, or binds at least in part, to at least a portion of the target protein selected from the portion consisting of:
a) amino acids 54-406 of MYC (SEQ ID NO: 1),
b) amino acids 354-406 of MYC (SEQ ID NO: 1),
c) amino acids 408-437 of MYC (SEQ ID NO: 1),
d) amino acids 410-419 of MYC (SEQ ID NO: 1),
c) amino acids 150-663 of CTNNB1 (SEQ ID NO: 3),
f) amino acids 15-29 of CTNNB1 (SEQ ID NO: 3),
g) amino acids 249-265, 292-306, 379-390, 415-429, 462-470, or 505-519, or any combination thereof, of CTNNB1 (SEQ ID NO: 3), and
h) amino acids 38-48, 123-129, or 251-257 or any combination thereof, of PCNA (SEQ ID NO: 2151).

In some embodiments, the first and/or second targeting moiety is selected from Omomyc, TCF4, TCF4.1, TCF4.2, TCF4.3, TCF4.4, TCF4.5, TCF4.6, TCF4.7, TCF4.8, TCF4.9, TCF4.10, TCF4.11, TCF4.12, TCF4.13, TCF4.14, TCF4.15, TCF4.16, con1.1, con1.2, p21.1, p21.2, p21.3, p21.4, p21.5, p21.6, p21.7, p21.8, p21.9, mycV.0300, mycV.1300, mycV.1500, mycV.3700, mycV.6600, mycV.1516, mycV.1515, mycV.1514, mycV.1513, mycV.1512, mycV.1511, mycV.1510, mycV.1509, mycV.1508, mycV.1507, mycV.1506, mycV.1505, mycV.1504, mycV.1503, mycV.1502, mycV.1501, or a fragment thereof that is capable of binding to the target protein. In some embodiments, the first and/or second targeting moiety does not comprise full length or unmodified con1 or p21. Exemplary sequences of first and/or second targeting moieties are found in Table 5. In some embodiments, the first and/or second targeting moiety comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NOs: 6-12, 1918, 1926, 1934, 1942, 1950, 1958, 1966, 1974, 1982, 1990, 1998, 2006, 2014, 2022, 2030, 2038, 2046, 2054, 2062, 2070, 2078, 2086, and 2087-2112. In some embodiments where the amino acid sequence differs from a recited reference sequence, the difference is the result of the inclusion of one or more modifications relative to the reference sequence. In some embodiments, the one or more modifications comprise a substitution, insertion and/or deletion. In some embodiments, the substitution is a conservative substitution. In some embodiments, the % sequence identity is calculated over the entirety of the reference sequence.

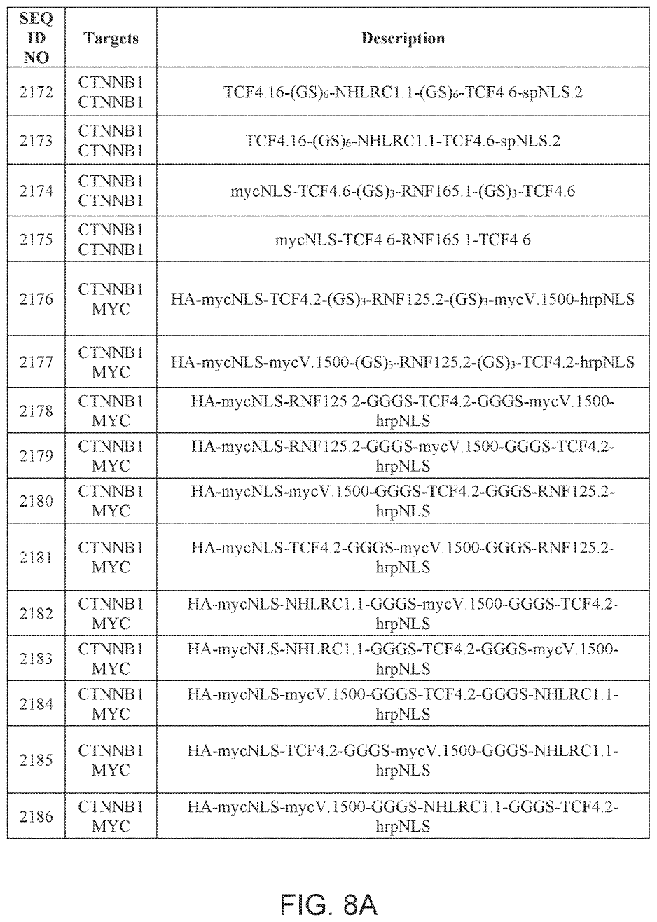

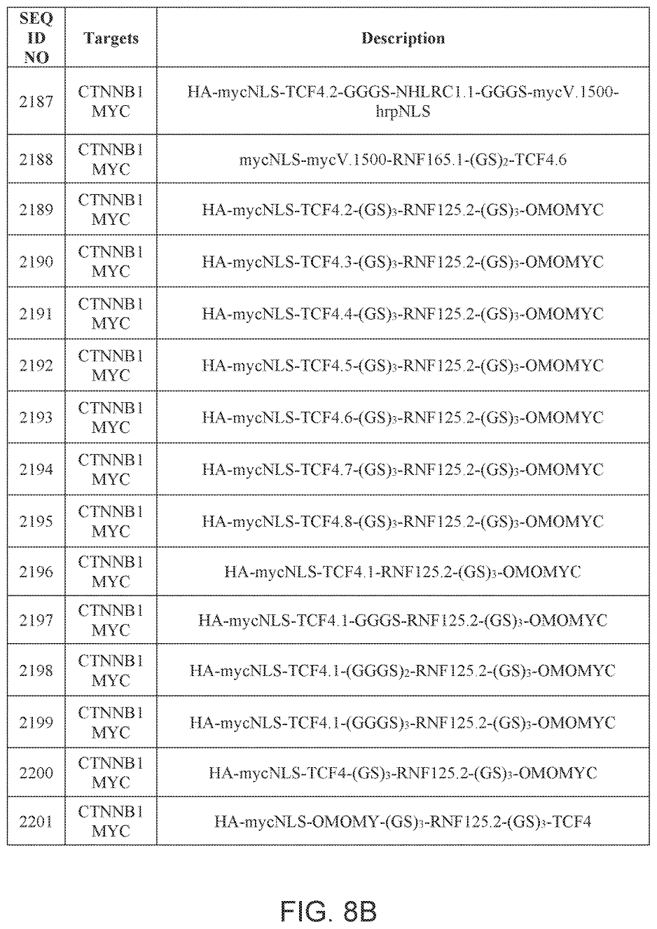

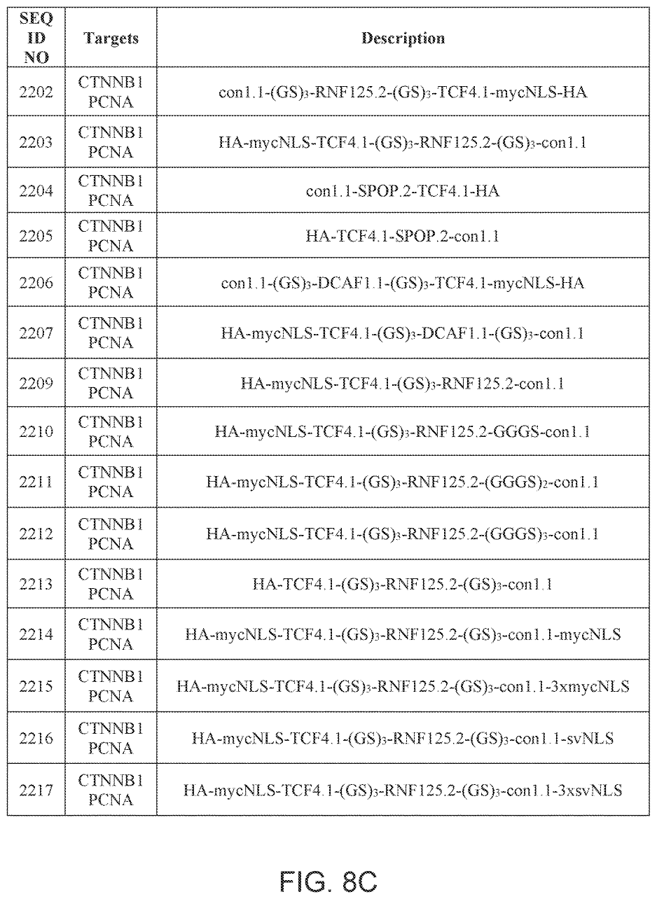

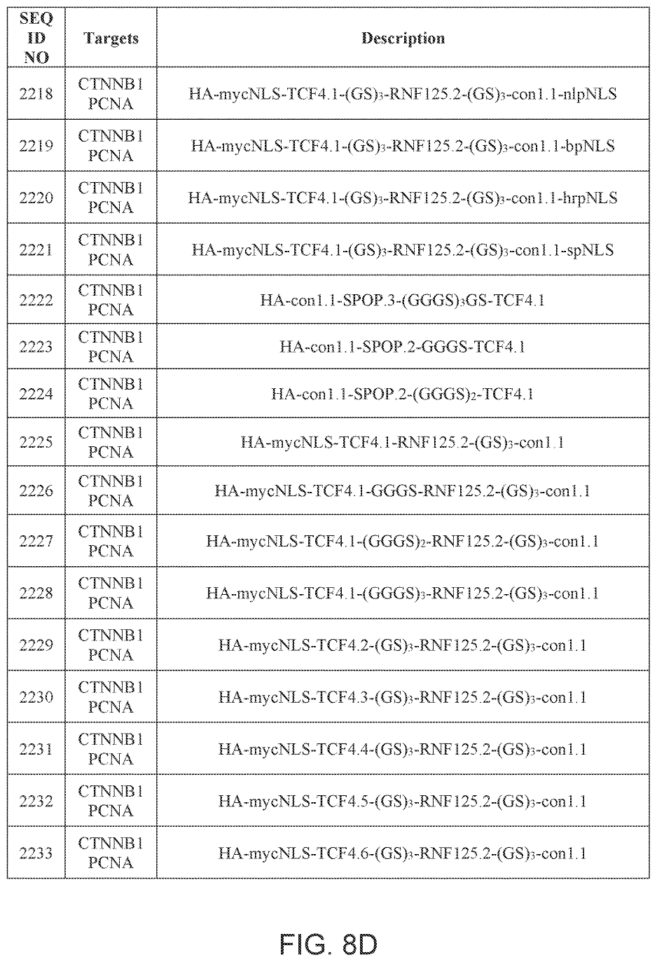

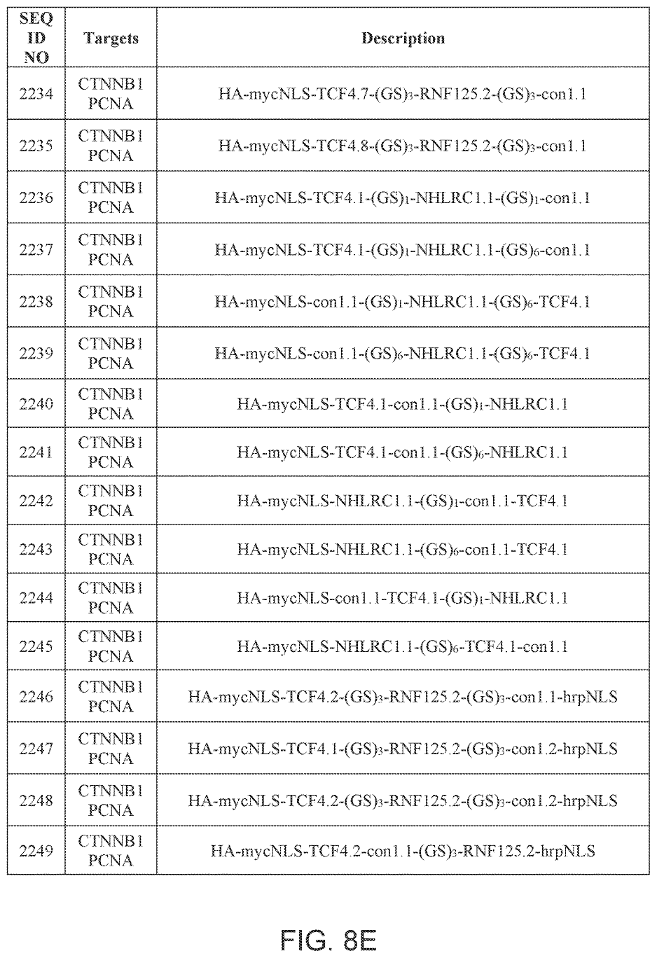

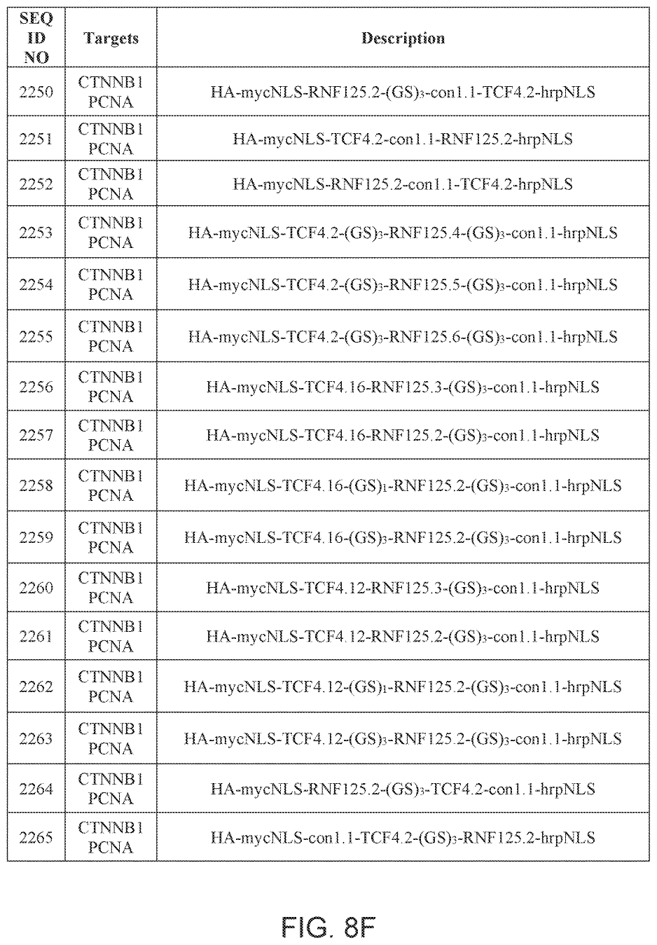

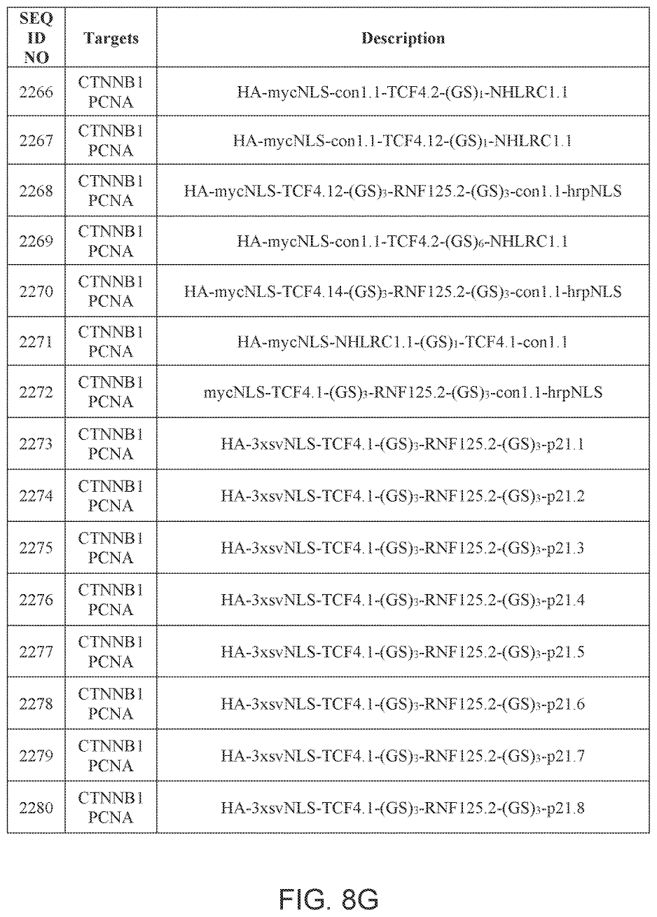

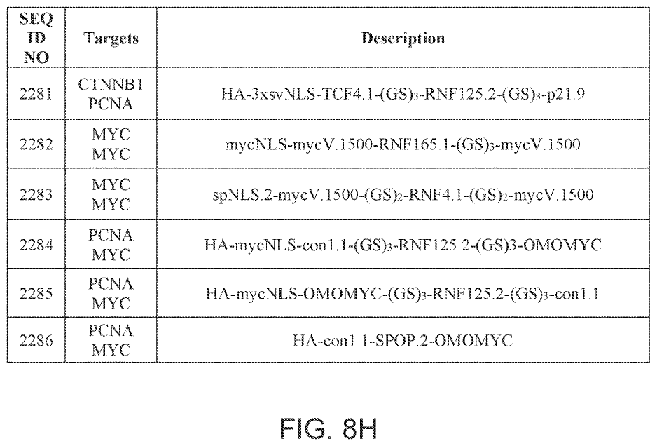

FIG. 8A-8H discloses embodiments of dual-specific bifunctional polypeptides. The description identifies the elements of the polypeptide in the N- to C-terminal direction. In some embodiments, the dual-specific bifunctional polypeptide is one of the embodiments described in FIG. 8A-8H. In some embodiments, the dual-specific bifunctional polypeptide comprises the embodiments in FIG. 8A-8H sharing one or more elements or features. In some embodiments the shared element or feature is the target. In some embodiments the shared element or feature is the URD name (e.g., those in Table 2). In some embodiments the shared element or feature is protein from which the URD is derived (e.g., those listed in Table 1). In some embodiments the shared element or feature is one or more of the following: the target; the protein from which the URD is derived; the URD name; the type of targeting moiety (e.g., sdAb, scFv, polypeptide binder); the targeting moiety name (e.g., those in Table 5; note that “full” is not included in the names in FIG. 8A-8H); the NLS name (e.g. those in Tables 3 and 4); the linker name (e.g., those in Table 15); the position of the URD relative to the targeting moiety (upstream or downstream); and both the URD name and targeting moiety name. In some embodiments, the HA tag sequence (SEQ ID NO: 50) is excluded. In some embodiments, the dual-specific bifunctional polypeptide comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NOs: 2172-2207 and 2209-2351, or optionally 2172-2207 and 2209-2286, In some embodiments, the dual-specific bifunctional polypeptide comprises or consists of an amino acid sequence of any one of SEQ ID NOs: 2172-2207 and 2209-2351, or optionally 2172-2207 and 2209-2286. In some embodiments, the dual-specific bifunctional polypeptide comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of 2172-2207 and 2209-2351, or optionally 2172-2207 and 2209-2286, optionally wherein the sequence lacks the HA tag sequence (SEQ ID NO: 50). In some embodiments where the amino acid sequence differs from a recited reference sequence, the difference is the result of the inclusion of one or more modifications relative to the reference sequence. In some embodiments, the one or more modifications comprise a substitution, insertion and/or deletion. In some embodiments, the substitution is a conservative substitution. In some embodiments, the % sequence identity is calculated over the entirety of the reference sequence.

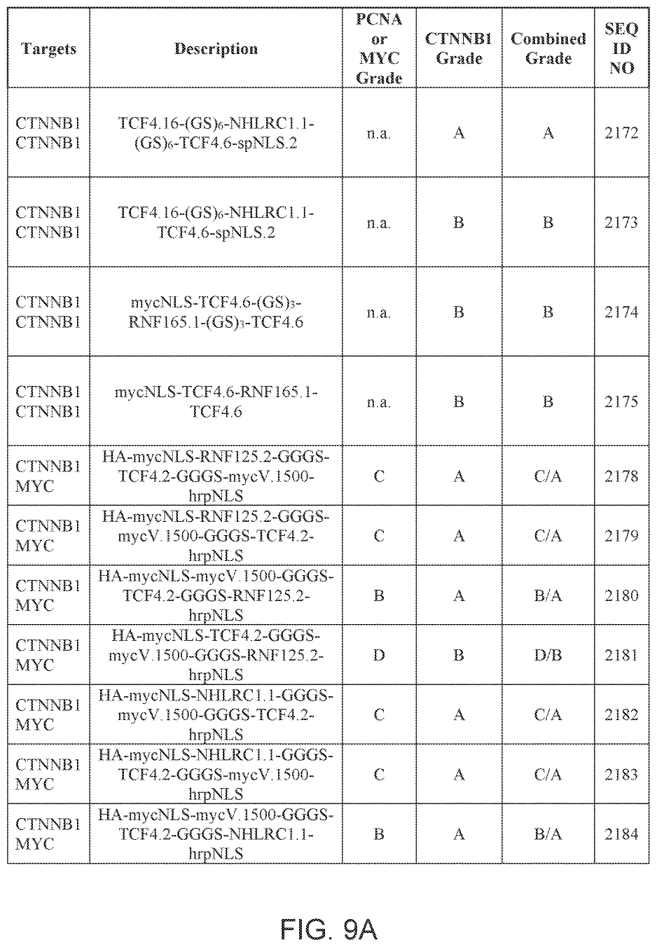

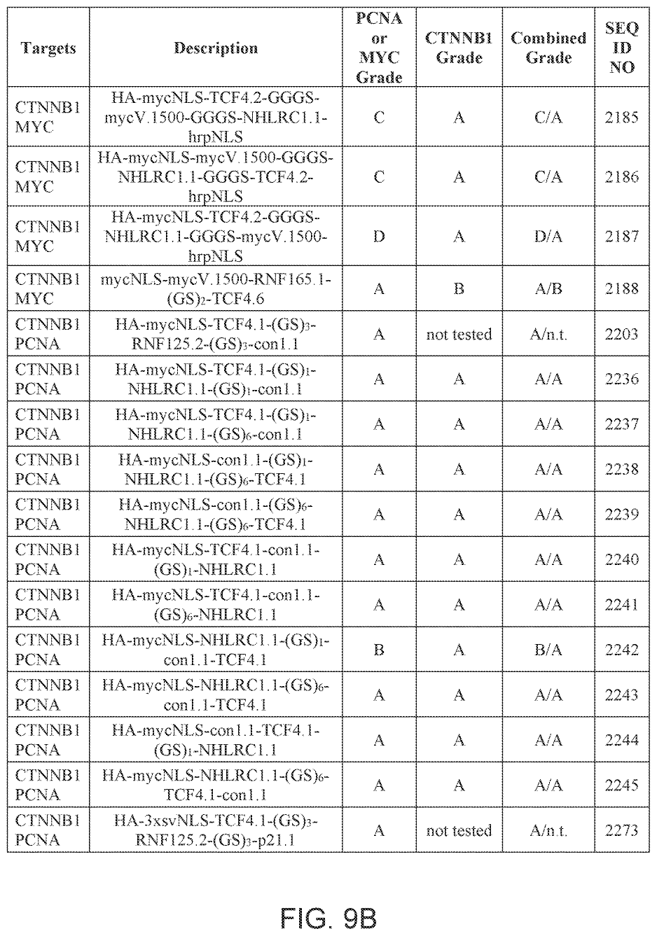

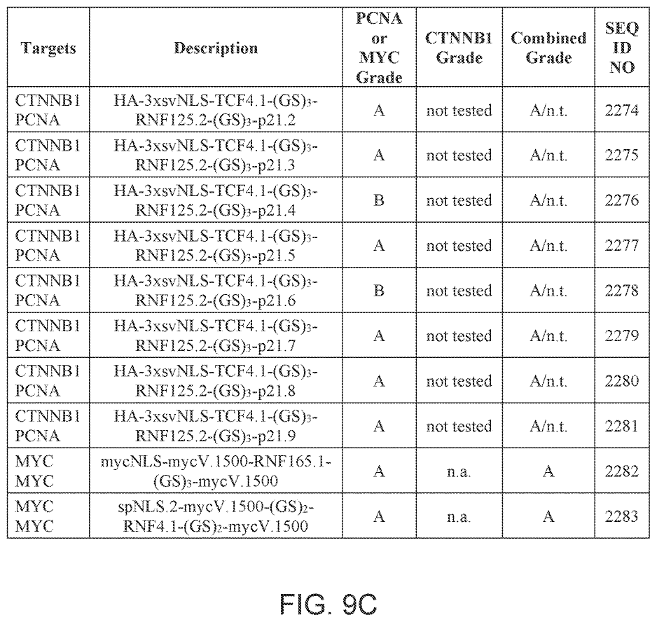

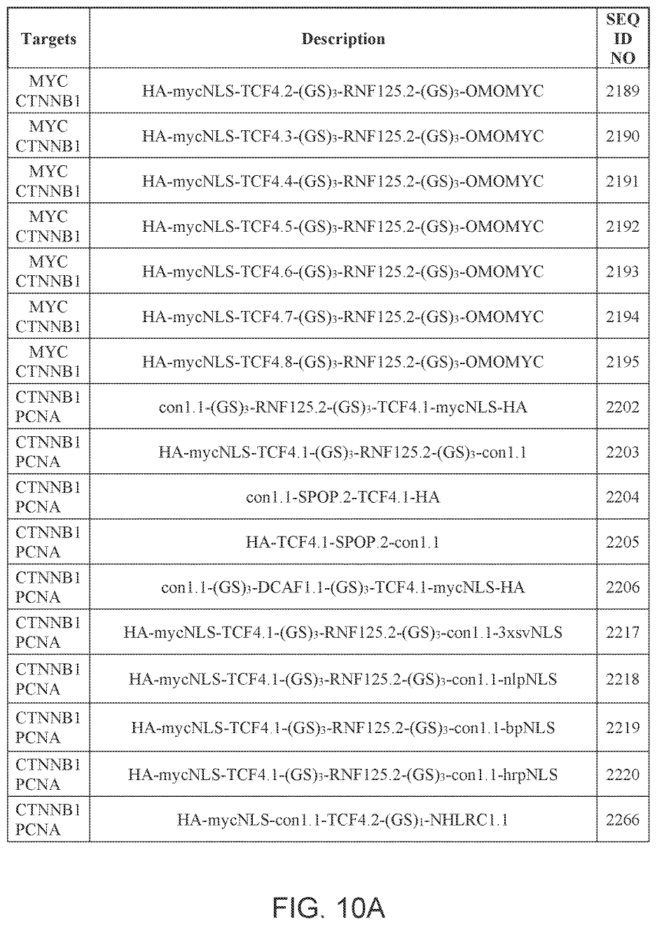

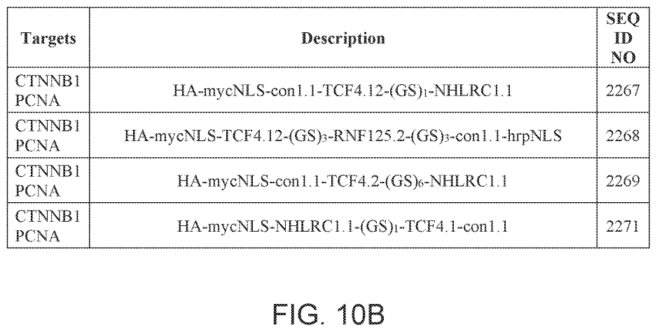

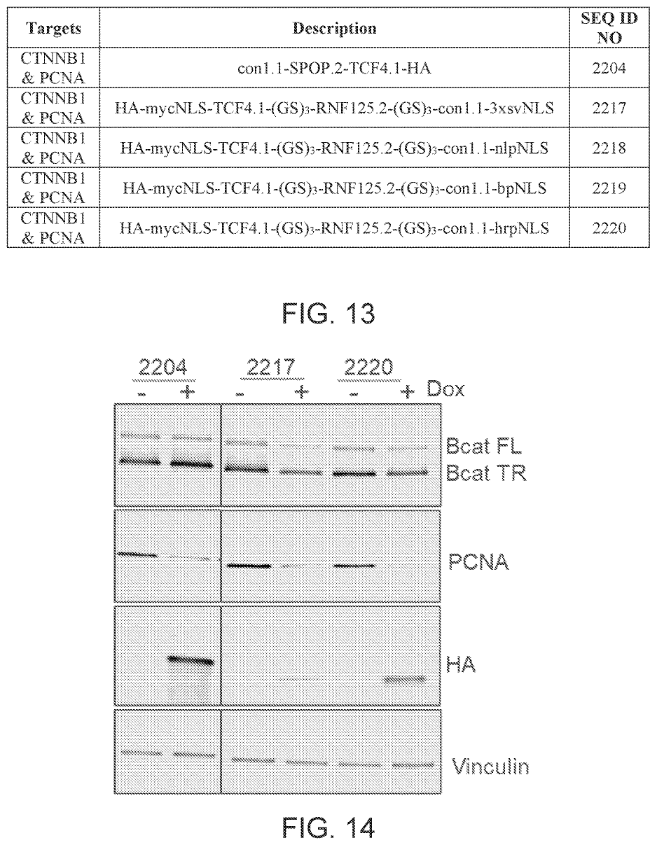

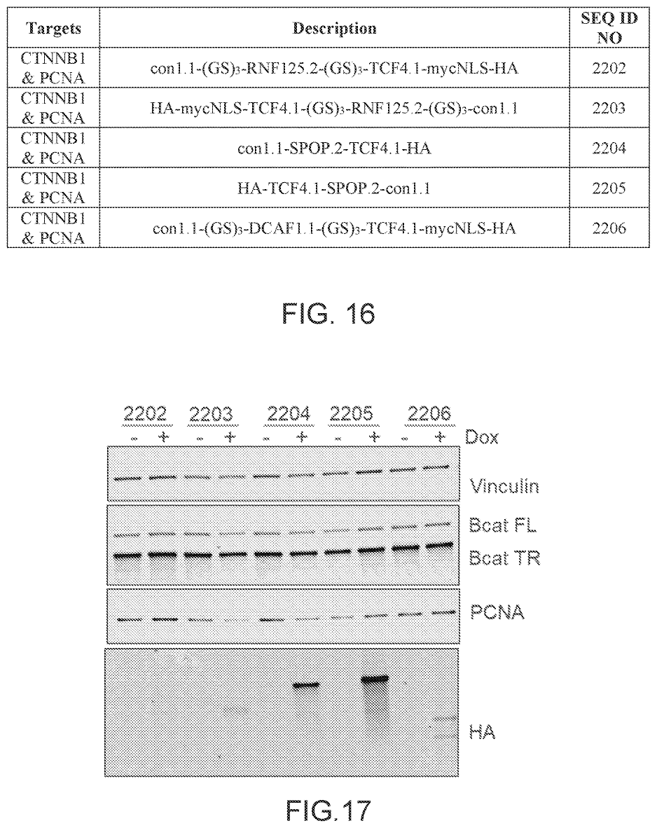

Provided below are tables listing the results of HiBiT™ degradation studies utilizing exemplary embodiments of dual-specific bifunctional polypeptides disclosed herein (Tables 7, 10, and 13, and FIG. 9A-C). The tables list a “Grade” of A, B, C or D for the level of degradation of each of the targeted proteins. For some of dual-specific bifunctional polypeptides disclosed in the tables, degradation of only one of the two target proteins was measured, and therefore only a grade is not reported for degradation of the other target protein. For dual-specific bifunctional polypeptides where the first and the second targeting moieties target the same protein, only a single grade is provided. Details regarding the experimental protocol are provided in the Examples section below. In some embodiments, the dual-specific bifunctional polypeptide comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of the SEQ ID NOs in any of the Tables disclosing Grade A degradation of at least one of the target proteins, Grade B degradation of at least one of the target proteins, Grade C degradation of at least one of the target proteins, and/or Grade D degradation of at least one of the target proteins, optionally wherein the sequence lacks the HA tag sequence (SEQ ID NO: 50). In some embodiments, the dual-specific bifunctional polypeptide comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of the SEQ ID NOs in any of the Tables disclosing Grade A degradation of at least one of the target proteins, optionally wherein the sequence lacks the HA tag sequence (SEQ ID NO: 50). In some embodiments, the dual-specific bifunctional polypeptide comprises or consists of an amino acid sequence of any one of the SEQ ID NOs in the Tables disclosing Grade A degradation of at least one of the target proteins, and/or Grade B degradation of at least one of the target proteins, optionally wherein the sequence lacks the HA tag sequence (SEQ ID NO: 50). In some embodiments, the dual-specific bifunctional polypeptide comprises or consists of an amino acid sequence of any one of the SEQ ID NOs in the Tables disclosing a Combined Grade A/A or A, optionally wherein the sequence lacks the HA tag sequence (SEQ ID NO: 50). In some embodiments, the dual-specific bifunctional polypeptide comprises or consists of an amino acid sequence of any one of the SEQ ID NOs in the Tables disclosing a Combined Grade A/B or B/A, optionally wherein the sequence lacks the HA tag sequence (SEQ ID NO: 50). In some embodiments, the dual-specific bifunctional polypeptide comprises or consists of an amino acid sequence of any one of the SEQ ID NOs in the Tables disclosing a Combined Grade B/B or B, optionally wherein the sequence lacks the HA tag sequence (SEQ ID NO: 50). In some embodiments, the dual-specific bifunctional polypeptide comprises or consists of an amino acid sequence of any one of the SEQ ID NOs in the Tables disclosing Grade A degradation of both target proteins, optionally wherein the sequence lacks the HA tag sequence (SEQ ID NO: 50). In some embodiments, the dual-specific bifunctional polypeptide does not comprise or consist of an amino acid sequence of any one of the SEQ ID NOs in Tables disclosing Grade C and/or D degradation of at least one of the target proteins, optionally wherein the sequence lacks the HA tag sequence (SEQ ID NO: 50). In some embodiments, the dual-specific bifunctional polypeptide comprises or consists of an amino acid sequence of any one of the SEQ ID NOs in Tables 8, 11, and 14, and FIGS. 10-19, optionally wherein the western blot shows degradation of one and/or both of the target proteins. In some embodiments, the dual-specific bifunctional polypeptide further shares one or more elements of features as described above and elsewhere herein. In some embodiments where the amino acid sequence differs from a recited reference sequence, the difference is the result of the inclusion of one or more modifications relative to the reference sequence. In some embodiments, the one or more modifications comprise a substitution, insertion and/or deletion. In some embodiments, the substitution is a conservative substitution. In some embodiments, the % sequence identity is calculated over the entirety of the reference sequence.

TABLE 5
Exemplary targeting moieties

| Target | Class | Name | SEQ ID NO |
|---|---|---|---|
| MYC | sdAb | mycV.0300 full | 1918 |
| MYC | sdAb | mycV.1300 full | 1926 |
| MYC | sdAb | mycV.1500 full | 1934 |
| MYC | sdAb | mycV.3700 full | 1942 |
| MYC | sdAb | mycV.6600 full | 1950 |
| MYC | sdAb | mycV.1516 full | 1958 |
| MYC | sdAb | mycV.1515 full | 1966 |
| MYC | sdAb | mycV.1514 full | 1974 |
| MYC | sdAb | mycV.1513 full | 1982 |
| MYC | sdAb | mycV.1512 full | 1990 |
| MYC | sdAb | mycV.1511 full | 1998 |
| MYC | sdAb | mycV.1510 full | 2006 |
| MYC | sdAb | mycV.1509 full | 2014 |
| MYC | sdAb | mycV.1508 full | 2022 |
| MYC | sdAb | mycV.1507 full | 2030 |
| MYC | sdAb | mycV.1506 full | 2038 |
| MYC | sdAb | mycV.1505 full | 2046 |
| MYC | sdAb | mycV.1504 full | 2054 |
| MYC | sdAb | mycV.1503 full | 2062 |
| MYC | sdAb | mycV.1502 full | 2070 |
| MYC | sdAb | mycV.1501 full | 2078 |
| MYC | peptide | OMOMYC | 2086 |
| CTNNB1 | peptide | TCF4.1 | 2087 |
| CTNNB1 | peptide | TCF4.10 | 2088 |
| CTNNB1 | peptide | TCF4.11 | 2089 |
| CTNNB1 | peptide | TCF4.12 | 2090 |
| CTNNB1 | peptide | TCF4.14 | 2091 |
| CTNNB1 | peptide | TCF4.15 | 2092 |
| CTNNB1 | peptide | TCF4.16 | 2093 |
| CTNNB1 | peptide | TCF4.2 | 2094 |
| CTNNB1 | peptide | TCF4.3 | 2095 |
| CTNNB1 | peptide | TCF4.4 | 2096 |
| CTNNB1 | peptide | TCF4.5 | 2097 |
| CTNNB1 | peptide | TCF4.6 | 2098 |
| CTNNB1 | peptide | TCF4.7 | 2099 |
| CTNNB1 | peptide | TCF4.8 | 2100 |
| CTNNB1 | peptide | TCF4.9 | 2101 |
| PCNA | peptide | con1.1 | 2102 |
| PCNA | peptide | con1.2 | 2103 |
| PCNA | peptide | p21.1 | 2104 |
| PCNA | peptide | p21.2 | 2105 |
| PCNA | peptide | p21.3 | 2106 |
| PCNA | peptide | p21.4 | 2107 |
| PCNA | peptide | p21.5 | 2108 |
| PCNA | peptide | p21.6 | 2109 |
| PCNA | peptide | p21.7 | 2110 |
| PCNA | peptide | p21.8 | 2111 |
| PCNA | peptide | p21.9 | 2112 |

### Exemplary Dual-Specific Bifunctional Degraders Targeting CTNNB1

In some embodiments, the target protein is beta catenin 1 (CTNNB1). In some embodiments, the CTNNB1 is a mutant form. An example sequence for CTNNB1 is provided as SEQ ID NO: 3 (Uniprot #P35222). CTNNB1 is involved in the pathology of various solid tumors (including but not limited to liver metastases, colorectal carcinoma, or other gastrointestinal cancers) and hematological malignancies. In some embodiments disclosed herein, the dual-specific bifunctional polypeptide comprises a first and/or second targeting moiety that is capable of binding to CTNNB1. Some examples of targeting moieties that target CTNNB1 include TCF4 (SEQ ID NO: 12; Uniprot #Q9NQB0), and amino acids 10-54 of TCF4 (SEQ ID NO: 11), although other suitable CTNNB1 targeting moieties generally known in the art may also be used. In some embodiments, the first and/or second targeting moieties that are capable of binding to CTNNB1 may be capable of binding, or binding at least in part, to at least a portion of CTNNB1, such as the armadillo domain of CTNNB1. In some embodiments, the first and/or second targeting moieties that are capable of binding to CTNNB1 are capable of binding, or binding at least in part, to at least a portion of amino acids 150-663 (the armadillo domain of CTNNB1; as seen in PDB: 2GL7), amino acids 15-29 (binding epitope of antibody BC2), or amino acids 249-265, 292-306, 379-390, 415-429, 462-470, or 505-519, (which are residues in the armadillo domain that interact with TCF4, E-cadherin, Axin, APC, TCF/LEF family transcription factors and/or SOX9) or any combination thereof, of CTNNB1 (as represented as SEQ ID NO: 3). In some embodiments only one of the two targeting moieties (the first or second; e.g., an N-TBD or a C-TBD) is capable of binding CTNNB1. In some embodiments, both of the target binding moieties (the first and second; e.g., N-TBD and C-TBD, two N-TBDs, or two C-TBDs) are capable of binding CTNNB1. The dual-specific bifunctional polypeptides disclosed herein targeting CTNNB1 may be used for the treatment of a cancer, for example, a cancer involving nuclear expression of beta-catenin and/or harboring mutations or alterations in the Wnt/beta-catenin signaling pathway, for example, solid tumors (including but not limited to liver metastases, colorectal carcinoma, or other gastrointestinal cancers) and hematological malignancies.

In some embodiments, the CTNNB1 target protein is a mutant form of CTNNB1 (SEQ ID NO: 3) comprising one or more mutations selected from: D32Y; D32N; D32V; D32G; S33Y; S33L; S33F; S33C; S33P; G34V; G34E; 135S; H36P; S37C; S37P; S37P and D207G; T41A; T41I; T42_K49del; S45F and Y670X wherein X indicates termination; S45C; S45F; S45P; S45Y; and S45del.

In some embodiments, the dual-specific bifunctional polypeptide targeting CTNNB1 comprises a localization peptide sequence which directs the dual-specific bifunctional polypeptide to the nucleus of a cell. In some embodiments, the localization peptide sequence comprises or consists of a NLS sequence selected from the group of the NLS sequences of MYC, SPOP, Hrp1, SV40, 53BP1, and nucleoplasmin. In some embodiments, the localization peptide sequence comprises or consists of a NLS sequence selected from the group of the NLS sequence of MYC, optionally mycNLS and/or 3xmycNLS, SPOP, optionally spNLS and/or spNLS.2, SV40, optionally 3xsvNLS, 53BP1 and Hrp1 In some embodiments, the NLS comprises or consists of a sequence selected from any one of SEQ ID NOs: 51, 1896-1909. In some embodiments, the NLS comprises or consists of a sequence selected from any one of SEQ ID NOs: 1896-1899, 1901 and 1905.

In some embodiments, the dual-specific bifunctional polypeptide targeting CTNNB1 comprises a URD derived from a URD selected from the group consisting of CBL-b (Y363E), CHIP, DCAF1, E6AP, FBXW7-alpha, Keap1, NHLRC1, RNF4, RNF12, RNF114, RNF125, RNF138, RNF166, SPOP, beta-TRCP, TRIM21, and VIF. In some embodiments, the URD is the URD of CHIP, DCAF1, E6AP, FBXW7a, FBXW7b, beta-TRCP, Keap1, RNF114, RNF125, RNF138, RNF165, NHLRC1, CBL-b (Y363E), TRIM21, SPOP, or VIF, or a truncation or fragment thereof that retains ubiquitin-proteasome recruiting activity, optionally RNF125.2, SPOP.2 or SPOP.3. In some embodiments, the URD is derived from NHLRC1, RNF125, or SPOP. In some embodiments, the dual-specific bifunctional polypeptide targeting CTNNB1 comprises a URD derived from a URD selected from the group consisting of DCAF1, NHLRC1, RNF125, RNF165, and SPOP. In some embodiments, the dual-specific bifunctional polypeptide targeting CTNNB1 comprises a URD derived from a URD selected from the group consisting of NHLRC1, RNF125, RNF165, and SPOP. In some embodiments, the URD comprises or consists of DCAF1.1, NHLRC1.1, RNF125.2, RNF125.3, RNF125.4, RNF125.5, RNF125.6, RNF165.1, SPOP.2 or SPOP.3. In some embodiments, the URD comprises or consists of NHLRC1.1, RNF125.2, RNF125.3, RNF125.4, RNF125.5, RNF125.6, RNF165.1, SPOP.2 or SPOP.3. In some embodiments, the URD is a truncation or fragment thereof that retains ubiquitin-proteasome recruiting activity. In some embodiments, the URD is truncation or fragment thereof that retains ubiquitin-proteasome recruiting activity. In some embodiments, the URD comprises or consists of a sequence selected from any one of SEQ ID NOs: 29-43, and 1836-1895. In some embodiments, the URD comprises or consists of a sequence selected from any one of SEQ ID NOs: 1837, 1855, 1856, 1858, 1870-1874, and 1880. In some embodiments, the URD comprises or consists of a sequence selected from any one of SEQ ID NOs: 1855, 1856, 1858, 1870-1874, and 1880

In some embodiments, the dual-specific bifunctional polypeptide targeting CTNNB1 comprises a first and/or second targeting moiety wherein the first and/or second targeting moiety comprises or consists of a targeting moiety disclosed in Table 6. In some embodiments, the first and/or second targeting moiety comprises or consists of an endogenous protein binding partner of the target protein, a fragment and/or a derivative thereof. In some embodiments, In some embodiments, the first and/or second targeting moiety comprises or consists of TCF4.1, TCF4.2, TCF4.3, TCF4.4, TCF4.5, TCF4.6, TCF4.7, TCF4.8, TCF4.9, TCF4.10, TCF4.11, TCF4.12, TCF4.13, TCF4.14, TCF4.15, or TCF4.16, or a fragment thereof that is capable of binding to the target protein. In some embodiments, the first and/or second targeting moiety comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NOs: 2087-2101, optionally any one of SEQ ID NOs: 2087, 2090, 2091, and 2093-2100. In some embodiments where the amino acid sequence differs from a recited reference sequence, the difference is the result of the inclusion of one or more modifications relative to the reference sequence. In some embodiments, the one or more modifications comprise a substitution, insertion and/or deletion. In some embodiments, the substitution is a conservative substitution. In some embodiments, the % sequence identity is calculated over the entirety of the reference sequence.

In some embodiments, the dual-specific bifunctional polypeptide targeting CTNNB1 comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NOs: 2172-2207 and 2209-2281, optionally wherein the sequence lacks the HA tag sequence (SEQ ID NO: 50). In some embodiments, the dual-specific bifunctional polypeptide targeting CTNNB1 comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NOs: 2189-2195, 2202-2206, 2217-2220, 2266-2269 and 2271 (SEQ ID NOs found in Table 8), optionally wherein the sequence lacks the HA tag sequence (SEQ ID NO: 50). In some embodiments, the dual-specific bifunctional polypeptide comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of the SEQ ID NOs in Table 7, optionally excluding any one of the SEQ ID NOs having a Grade of D degradation of CTNNB1 and/or another target protein, optionally wherein the sequence lacks the HA tag sequence (SEQ ID NO: 50). In some embodiments, the dual-specific bifunctional polypeptide comprises or consists of an amino acid sequence of any one of the SEQ ID NOs in Table 7 having a Grade A degradation of CTNNB1, and/or a Grade B degradation of CTNNB1, optionally wherein the sequence lacks the HA tag sequence (SEQ ID NO: 50). In some embodiments, the dual-specific bifunctional polypeptide comprises or consists of an amino acid sequence of any one of the SEQ ID NOs in Table 7 having a Combined Grade of A and/or A/A, optionally wherein the sequence lacks the HA tag sequence (SEQ ID NO: 50). In some embodiments, the dual-specific bifunctional polypeptide comprises or consists of an amino acid sequence of any one of the SEQ ID NOs in Table 7 having a Combined Grade of A/B and/or B/A, optionally wherein the sequence lacks the HA tag sequence (SEQ ID NO: 50). In some embodiments, the dual-specific bifunctional polypeptide comprises or consists of an amino acid sequence of any one of the SEQ ID NOs in Table 8, optionally wherein the western blot analysis depicted in any one of FIG. 12, 14, 15, 17 or 19 demonstrate degradation of one or both of the target proteins. In some embodiments, the dual-specific bifunctional polypeptide does not comprise or consist of an amino acid sequence of any one of the SEQ ID NOs in Table 7 having Grades C and/or D for either target protein, optionally wherein the sequence lacks the HA tag sequence (SEQ ID NO: 50). In some embodiments where the amino acid sequence differs from a recited reference sequence, the difference is the result of the inclusion of one or more modifications relative to the reference sequence. In some embodiments, the one or more modifications comprise a substitution, insertion and/or deletion. In some embodiments, the substitution is a conservative substitution. In some embodiments, the % sequence identity is calculated over the entirety of the reference sequence.

In some embodiments, the dual-specific bifunctional polypeptide targeting CTNNB1 comprises embodiments further sharing one or more elements or features. In some embodiments the shared element or feature is the URD name. In some embodiments the shared element or feature is protein from which the URD is derived. In some embodiments the shared element or feature is one or more of the following: the protein from which the URD is derived; the URD name; the type of targeting moiety; the targeting moiety name; the NLS name; the linker name; the position of the URD relative to the targeting moiety (upstream or downstream); and both the URD name and targeting moiety name. In some embodiments, the HA tag sequence (SEQ ID NO: 50) is excluded.

In some embodiments of the dual-specific bifunctional polypeptide targeting CTNNB1 the URD is not derived from beta-TRCP. In some embodiments, the URD does not comprise or consist of bTRCP.1. In some embodiments, the URD does not comprise or consist of SEQ ID NOs: 1839

In some embodiments, disclosed herein is a polynucleotide encoding any of the dual-specific bifunctional polypeptides targeting CTNNB1 disclosed above, or elsewhere herein.

TABLE 6
Exemplary CTNNB1 Targeting Moieties

| SEQ ID NO | Category | Name |
|---|---|---|
| 2112 | peptide | TCF4.1 |
| 2113 | peptide | TCF4.10 |
| 2114 | peptide | TCF4.11 |
| 2115 | peptide | TCF4.12 |
| 2116 | peptide | TCF4.14 |
| 2117 | peptide | TCF4.15 |
| 2118 | peptide | TCF4.16 |
| 2119 | peptide | TCF4.2 |
| 2120 | peptide | TCF4.3 |
| 2121 | peptide | TCF4.4 |
| 2122 | peptide | TCF4.5 |
| 2123 | peptide | TCF4.6 |
| 2124 | peptide | TCF4.7 |
| 2125 | peptide | TCF4.8 |
| 2126 | peptide | TCF4.9 |

TABLE 7
Exemplary dual-specific bifunctional polypeptides targeting CTNNB1 and
corresponding exemplary target protein degradation grade

| | | | PCNA | | |
|---|---|---|---|---|---|
| | | | or | | SEQ |
| | | CTNNB1 | MYC | Combined | ID |
| Targets | Description | Grade | Grade | Grade | NO |
| CTNNB1 | HA-mycNLS-TCF4.1-(GS)₁- | A | A | A/A | 2236 |
| PCNA | NHLRC1.1-(GS)₁-con1.1 | | | | |
| CTNNB1 | HA-mycNLS-TCF4.1-(GS)₁- | A | A | A/A | 2237 |
| PCNA | NHLRC1.1-(GS)₆-con1.1 | | | | |
| CTNNB1 | HA-mycNLS-con1.1-(GS)₁- | A | A | A/A | 2238 |
| PCNA | NHLRC1.1-(GS)₆-TCF4.1 | | | | |
| CTNNB1 | HA-mycNLS-con1.1-(GS)₆- | A | A | A/A | 2239 |
| PCNA | NHLRC1.1-(GS)₆-TCF4.1 | | | | |
| CTNNB1 | HA-mycNLS-TCF4.1-con1.1- | A | A | A/A | 2240 |
| PCNA | (GS)₁-NHLRC1.1 | | | | |
| CTNNB1 | HA-mycNLS-TCF4.1-con1.1- | A | A | A/A | 2241 |
| PCNA | (GS)₆-NHLRC1.1 | | | | |
| CTNNB1 | HA-mycNLS-NHLRC1.1-(GS)₆- | A | A | A/A | 2243 |
| PCNA | con1.1-TCF4.1 | | | | |
| CTNNB1 | HA-mycNLS-con1.1-TCF4.1- | A | A | A/A | 2244 |
| PCNA | (GS)₁-NHLRC1.1 | | | | |
| CTNNB1 | HA-mycNLS-NHLRC1.1-(GS)₆- | A | A | A/A | 2245 |
| PCNA | TCF4.1-con1.1 | | | | |
| CTNNB1 | HA-mycNLS-NHLRC1.1-(GS)₁- | A | B | A/B | 2242 |
| PCNA | con1.1-TCF4.1 | | | | |
| CTNNB1 | HA-mycNLS-mycV.1500- | A | B | A/B | 2180 |
| MYC | GGGS-TCF4.2-GGGS- | | | | |
| | RNF125.2-hrpNLS | | | | |
| CTNNB1 | HA-mycNLS-mycV.1500- | A | B | A/B | 2184 |
| MYC | GGGS-TCF4.2-GGGS- | | | | |
| | NHLRC1.1-hrpNLS | | | | |
| CTNNB1 | HA-mycNLS-RNF125.2-GGGS- | A | C | A/C | 2178 |
| MYC | TCF4.2-GGGS-mycV.1500- | | | | |
| | hrpNLS | | | | |
| CTNNB1 | HA-mycNLS-RNF125.2-GGGS- | A | C | A/C | 2179 |
| MYC | mycV.1500-GGGS-TCF4.2- | | | | |
| | hrpNLS | | | | |
| CTNNB1 | HA-mycNLS-NHLRC1.1-GGGS- | A | C | A/C | 2182 |
| MYC | mycV.1500-GGGS-TCF4.2- | | | | |
| | hrpNLS | | | | |
| CTNNB1 | HA-mycNLS-NHLRC1.1-GGGS- | A | C | A/C | 2183 |
| MYC | TCF4.2-GGGS-mycV.1500- | | | | |
| | hrpNLS | | | | |
| CTNNB1 | HA-mycNLS-TCF4.2-GGGS- | A | C | A/C | 2185 |
| MYC | mycV.1500-GGGS-NHLRC1.1- | | | | |
| | hrpNLS | | | | |
| CTNNB1 | HA-mycNLS-mycV.1500- | A | C | A/C | 2186 |
| MYC | GGGS-NHLRC1.1-GGGS- | | | | |
| | TCF4.2-hrpNLS | | | | |
| CTNNB1 | HA-mycNLS-TCF4.2-GGGS- | A | D | A/D | 2187 |
| MYC | NHLRC1.1-GGGS-mycV.1500- | | | | |
| | hrpNLS | | | | |
| CTNNB1 | TCF4.16-(GS)₆-NHLRC1.1- | A | n.a. | A | 2172 |
| CTNNB1 | (GS)₆-TCF4.6-spNLS.2 | | | | |
| CTNNB1 | mycNLS-mycV.1500-RNF165.1- | B | A | B/A | 2188 |
| MYC | (GS)₂-TCF4.6 | | | | |
| CTNNB1 | TCF4.16-(GS)₆-NHLRC1.1- | B | n.a. | B | 2173 |
| CTNNB1 | TCF4.6-spNLS.2 | | | | |
| CTNNB1 | mycNLS-TCF4.6-(GS)₃- | B | n.a. | B | 2174 |
| CTNNB1 | RNF165.1-(GS)₃-TCF4.6 | | | | |
| CTNNB1 | mycNLS-TCF4.6-RNF165.1- | B | n.a. | B | 2175 |
| CTNNB1 | TCF4.6 | | | | |
| CTNNB1 | HA-mycNLS-TCF4.2-GGGS- | B | D | B/D | 2181 |
| MYC | mycV.1500-GGGS-RNF125.2- | | | | |
| | hrpNLS | | | | |

TABLE 8
Exemplary dual-specific bifunctional polypeptides targeting MYC with
exemplary western blot data

| | | SEQ |
|---|---|---|
| | | ID |
| Targets | Description | NO |
| CTNNB1 | HA-mycNLS-TCF4.2-(GS)₃-RNF125.2-(GS)₃-OMOMYC | 2189 |
| MYC | | |
| CTNNB1 | HA-mycNLS-TCF4.3-(GS)₃-RNF125.2-(GS)₃-OMOMYC | 2190 |
| MYC | | |
| CTNNB1 | HA-mycNLS-TCF4.4-(GS)₃-RNF125.2-(GS)₃-OMOMYC | 2191 |
| MYC | | |
| CTNNB1 | HA-mycNLS-TCF4.5-(GS)₃-RNF125.2-(GS)₃-OMOMYC | 2192 |
| MYC | | |
| CTNNB1 | HA-mycNLS-TCF4.6-(GS)₃-RNF125.2-(GS)₃-OMOMYC | 2193 |
| MYC | | |
| CTNNB1 | HA-mycNLS-TCF4.7-(GS)₃-RNF125.2-(GS)₃-OMOMYC | 2194 |
| MYC | | |
| CTNNB1 | HA-mycNLS-TCF4.8-(GS)₃-RNF125.2-(GS)₃-OMOMYC | 2195 |
| MYC | | |
| CTNNB1 | con1.1-(GS)₃-RNF125.2-(GS)₃-TCF4.1-mycNLS-HA | 2202 |
| PCNA | | |
| CTNNB1 | HA-mycNLS-TCF4.1-(GS)₃-RNF125.2-(GS)₃-con1.1 | 2203 |
| PCNA | | |
| CTNNB1 | con1.1-SPOP.2-TCF4.1-HA | 2204 |
| PCNA | | |
| CTNNB1 | HA-TCF4.1-SPOP.2-con1.1 | 2205 |
| PCNA | | |
| CTNNB1 | con1.1-(GS)₃-DCAF1.1-(GS)₃-TCF4.1-mycNLS-HA | 2206 |
| PCNA | | |
| CTNNB1 | HA-mycNLS-TCF4.1-(GS)₃-RNF125.2-(GS)₃-con1.1-3xsvNLS | 2217 |
| PCNA | | |
| CTNNB1 | HA-mycNLS-TCF4.1-(GS)₃-RNF125.2-(GS)₃-con1.1-nlpNLS | 2218 |
| PCNA | | |
| CTNNB1 | HA-mycNLS-TCF4.1-(GS)₃-RNF125.2-(GS)₃-con1.1-bpNLS | 2219 |
| PCNA | | |
| CTNNB1 | HA-mycNLS-TCF4.1-(GS)₃-RNF125.2-(GS)₃-con1.1-hrpNLS | 2220 |
| PCNA | | |
| CTNNB1 | HA-mycNLS-con1.1-TCF4.2-(GS)₃-NHLRC1.1 | 2266 |
| PCNA | | |
| CTNNB1 | HA-mycNLS-con1.1-TCF4.12-(GS)₃-NHLRC1.1 | 2267 |
| PCNA | | |
| CTNNB1 | HA-mycNLS-TCF4.12-(GS)₃-RNF125.2-(GS)₃-con1.1-hrpNLS | 2268 |
| PCNA | | |
| CTNNB1 | HA-mycNLS-con1.1-TCF4.2-(GS)₆-NHLRC1.1 | 2269 |
| PCNA | | |
| CTNNB1 | HA-mycNLS-NHLRC1.1-(GS)₁-TCF4.1-con1.1 | 2271 |
| PCNA | | |

### Exemplary Dual-Specific Bifunctional Degraders Targeting MYC

One exemplary target protein embodied herein is MYC proto-oncogene, bHLH transcription factor (MYC; c-MYC). In some embodiments, the MYC is a mutant form. An example sequence for MYC is provided as SEQ ID NO: 1 (Uniprot #P01106). MYC is a major oncogene involved in the pathology of many types of cancer, including solid tumors (including but not limited to hepatocellular carcinoma, liver metastases, and colorectal carcinoma) and hematological malignancies. In embodiments disclosed herein, the dual-specific bifunctional polypeptide comprises a first and/or second targeting moiety (also referred to as a “binder” or “binding moiety”) that is capable of binding to MYC. Some examples of first and/or second targeting moieties that target MYC include Omomyc (SEQ ID NO: 6), although other suitable targeting moieties that bind to MYC generally known in the art may also be used. In some embodiments, the first and/or second targeting moieties that are capable of binding to MYC may be capable of binding, or binding at least in part, to at least a portion of MYC, such as the DNA binding domain of MYC. In some embodiments, the first and/or second targeting moieties that are capable of binding to MYC are capable of binding, or binding at least in part, to at least a portion of amino acids 54-406, amino acids 354-406 (the DNA binding domain of MYC that binds to Omomyc; as seen in PDB: 1NKP), amino acids 408-437, or amino acids 410-419 (binding epitope of antibody 9E10; as seen in PDB: 20R9) of MYC (as represented as SEQ ID NO: 1). In some embodiments only one of the two targeting moieties (the first or second; e.g., an N-TBD or a C-TBD) is capable of binding MYC. In some embodiments, both of the target binding moieties (the first and second; e.g., N-TBD and C-TBD, two N-TBDs, or two C-TBDs) are capable of binding MYC. The dual-specific bifunctional polypeptides disclosed herein that are specific for MYC may be used for the treatment of a cancer, for example, a cancer exhibiting elevated levels of MYC, for example solid tumors (including but not limited to hepatocellular carcinoma, liver metastases, and colorectal carcinoma) and hematological malignancies.

In some embodiments, the bifunction polypeptide targeting MYC comprises a localization peptide sequence which directs the dual-specific bifunctional polypeptide to the nucleus of a cell. In some embodiments, the localization peptide sequence comprises or consists of the NLS sequence selected from the group of the NLS sequences of MYC, SPOP, Hrp1, SV40, and nucleoplasmin. In some, the localization peptide sequence comprises or consists of a NLS sequence selected from the group of the NLS sequence of SPOP, optionally SPNLS.2, MYC, optionally mycNLS, and Hrp1. In some embodiments, the NLS comprises or consists of a sequence selected from any one of SEQ ID NOs: 51, 1896-1909. In some embodiments, the NLS comprises or consists of a sequence selected from any one of SEQ ID NOs: 1904, 1896 and 1898.

In some embodiments, the dual-specific bifunctional polypeptide targeting MYC comprises a URD derived from a URD selected from the group consisting of CBL-b (Y363E), CHIP, DCAF1, E6AP, FBXW7-alpha, Keap1, NHLRC1, RNF4, RNF6, RNF11, RNF12, RNF20, RNF25, RNF111, RNF115, RNF125, RNF128, RNF149, RNF152, RNF165, RNF182, SPOP, TRIM21, TRIM32, VIF, ZNRF1, and ZNRF4. In some embodiments, the URD is a truncation or fragment thereof that retains ubiquitin-proteasome recruiting activity. In some embodiments, the URD is derived from CHIP, DCAF1, E6AP, FBXW7a, FBXW7b, beta-TRCP, Keap1, RNF114, RNF125, RNF138, NHLRC1, CBL-b (Y363E), TRIM21, SPOP or VIF. In some embodiments, the URD is derived from NHLRC1, RNF4, RNF125, RNF165, or SPOP. In some embodiments, the URD is a truncation or fragment thereof that retains ubiquitin-proteasome recruiting activity. In some embodiments, the URD comprises or consists of NHLRC1.1, RNF4.1, RNF125.2, RNF165.1, or SPOP.2. In some embodiments, the URD comprises or consists of NHLRC1.1, RNF4.1, RNF125.2, or RNF165.1. In some embodiments, the URD comprises or consists of RNF165.1, RNF4.1, or RNF125.2 In some embodiments, the URD comprises or consists of a sequence selected from any one of SEQ ID NOs: 1836-1838, 1840-1848, 1853-18-64, 1866-1876, 1878-1880, 1882-1895. In some embodiments, the URD comprises or consists of a sequence selected from any one of SEQ ID NOs: 29-42 and 1836-1895. In some embodiments, the URD comprises or consists of a sequence selected from any one of SEQ ID NOs: 1858, 1886, 1870, 1880, and 1855. In some embodiments, the URD comprises or consists of a sequence selected from any one of SEQ ID NOs: 1858, 1886, 1870, and 1880. In some embodiments, the URD comprises or consists of a sequence selected from any one of SEQ ID NOs: 1886, 1870, and 1880.

In some embodiments, the dual-specific bifunctional polypeptide targeting MYC comprises a first and/or second targeting moiety that comprises or consists of a targeting moiety disclosed in Table 9. In some embodiments, the first and/or second targeting moiety comprises or consists of an endogenous protein binding partner of the target protein, a fragment and/or a derivative thereof. In some embodiments, the first and/or second targeting moiety comprises or consists of Omomyc, or a fragment thereof that is capable of binding to the target protein. In some embodiments, the first and/or second targeting moiety comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to SEQ ID NO: 2086. In some embodiments where the amino acid sequence differs from a recited reference sequence, the difference is the result of the inclusion of one or more modifications relative to the reference sequence. In some embodiments, the one or more modifications comprise a substitution, insertion and/or deletion. In some embodiments, the substitution is a conservative substitution. In some embodiments, the % sequence identity is calculated over the entirety of the reference sequence.

In some embodiments, the dual-specific bifunctional polypeptide targeting MYC comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NOs: 2189-2201 and 2284-2286, optionally wherein the sequence lacks the HA tag sequence (SEQ ID NO: 50). In some embodiments, the dual-specific bifunctional polypeptide targeting MYC comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NOs: 2189-2193 (SEQ ID NOs listed in Table 11), optionally wherein the sequence lacks the HA tag sequence (SEQ ID NO: 50). In some embodiments where the amino acid sequence differs from a recited reference sequence, the difference is the result of the inclusion of one or more modifications relative to the reference sequence. In some embodiments, the one or more modifications comprise a substitution, insertion and/or deletion. In some embodiments, the substitution is a conservative substitution. In some embodiments, the % sequence identity is calculated over the entirety of the reference sequence.

In some embodiments, the dual-specific bifunctional polypeptide targeting MYC comprises a first and/or second targeting moiety that comprises or consists of a targeting moiety disclosed in Table 9. In some embodiments, the first and/or second targeting moiety comprises or consists of a sdAb, optionally comprising or consisting of either a Va domain or a V_{L} domain of an IgG antibody, or a V_{H}H, V_{H}, or V_{NAR} domain of a heavy chain antibody (HcAb). In some embodiments, the sdAb comprises or consists of a V_{H}H domain. In some embodiments, the first and/or second targeting moiety comprises or consists of a sdAb, optionally a V_{H}H domain, comprising a CDR1 selected from any one of SEQ ID NOs: 1920, 1928, 1944, and 1952, a CDR2 selected from any one of SEQ ID NOs: 1922, 1930, 1938, and 1946, and a CDR3 selected from any one of SEQ ID NOs: 1924, 1932, 1940, 1948, and 1956. In some embodiments, the combination of CDR1, CDR2, and CDR3 is 1936, 1938 and 1940. In some embodiments, each CDR sequence independently comprises 1, 2, 3, 4, 5, 6, 7, or more substitutions (optionally conservative substitutions) and/or 1, 2, 3, 4, 5, 6, 7, or more deletions or additions, and/or has at least 90%, 91%, 92%, 93%, 94%, 95%, 96%, 97%, 98%, 99%, or 100% sequence identity to a sequence selected from any one of SEQ ID NOs: 1920, 1928, 1944, 1952, 1922, 1930, 1938, 1946, 1924, 1932, 1940, 1948, and 1956. In some embodiments, the sdAb, optionally a V_{H}H domain, comprises a V_{H} FR1 selected from any one of SEQ ID NOs: 1919 and 1951, a V_{H} FR2 of SEQ ID NO: 1921, a V_{H} FR3 selected from any one of SEQ ID NOs: 1923, 1939, and 1955, and a V_{H} FR4 selected from any one of SEQ ID NOs: 1925 and 1933. In some embodiments, the combination of FR1, FR2, FR3 and FR4 is 1935, 1937, 1939, and 1941. In some embodiments, each V_{H} framework region comprises 1, 2, 3, 4, 5, 6, 7, 8, 9, 10, 11, 12, 13, 14, 15, 16, 17 or more substitutions (optionally conservative substitutions) and/or 1, 2, 3, 4, 5, 6, 7, 8, 9, 10, 11, 12, 13, 14, 15, 16, 17 or more deletions or additions, and/or has at least 90%, 91%, 92%, 93%, 94%, 95%, 96%, 97%, 98%, 99%, or 100% sequence identity to a sequence selected from any one of SEQ ID NOs: 1919, 1951, 1921, 1923, 1939, 1955, 1925, and 1933. In some embodiments, the first and/or second targeting moiety comprises or consists of a sdAb, optionally a V_{H}H domain, sequence selected from any one of SEQ ID NOs: 1918, 1926, 1934, 1942, 1950, 1958, 1966, 1974, 1982, 1990, 1998, 2006, 2014, 2022, 2030, 2038, 2046, 2054, 2062, 2070, and 2078. In some embodiments, each CDR sequence independently comprises 1, 2, 3, 4, 5, 6, 7, or more substitutions (optionally conservative substitutions) and/or 1, 2, 3, 4, 5, 6, 7, or more deletions or additions, and/or has at least 90%, 91%, 92%, 93%, 94%, 95%, 96%, 97%, 98%, 99%, or 100% sequence identity to a CDR sequence of any one of SEQ ID NOs: 1918, 1926, 1934, 1942, 1950, 1958, 1966, 1974, 1982, 1990, 1998, 2006, 2014, 2022, 2030, 2038, 2046, 2054, 2062, 2070, and 2078, and/or wherein the framework region comprises 1, 2, 3, 4, 5, 6, 7, 8, 9, 10, 11, 12, 13, 14, 15, 16, 17 or more substitutions (optionally conservative substitutions) and/or 1, 2, 3, 4, 5, 6, 7, 8, 9, 10, 11, 12, 13, 14, 15, 16, 17 or more deletions or additions, and/or has at least 90%, 91%, 92%, 93%, 94%, 95%, 96%, 97%, 98%, 99%, or 100% sequence identity to the framework region of any one of SEQ ID NOs: 1918, 1926, 1934, 1942, 1950, 1958, 1966, 1974, 1982, 1990, 1998, 2006, 2014, 2022, 2030, 2038, 2046, 2054, 2062, 2070, and 2078. In some embodiments where the amino acid sequence differs from a recited reference sequence, the difference is the result of the inclusion of one or more modifications relative to the reference sequence. In some embodiments, the one or more modifications comprise a substitution, insertion and/or deletion. In some embodiments, the substitution is a conservative substitution. In some embodiments, the % sequence identity is calculated over the entirety of the reference sequence.

In some embodiments, the dual-specific bifunctional polypeptide targeting MYC comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NOs: 2176-2188, and 2282-2283, optionally wherein the sequence lacks the HA tag sequence (SEQ ID NO: 50). In some embodiments, the dual-specific bifunctional polypeptide comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of the SEQ ID NOs in Table 10, optionally excluding any one of the SEQ ID NOs having a Grade of D degradation of MYC and/or another target protein, optionally wherein the sequence lacks the HA tag sequence (SEQ ID NO: 50). In some embodiments, the dual-specific bifunctional polypeptide comprises or consists of an amino acid sequence of any one of the SEQ ID NOs in Table 10 having a Grade A degradation of MYC, and/or a Grade B degradation of MYC, optionally wherein the sequence lacks the HA tag sequence (SEQ ID NO: 50). In some embodiments, the dual-specific bifunctional polypeptide comprises or consists of an amino acid sequence of any one of the SEQ ID NOs in Table 10 having a Combined Grade of A and/or A/A, optionally wherein the sequence lacks the HA tag sequence (SEQ ID NO: 50). In some embodiments, the dual-specific bifunctional polypeptide comprises or consists of an amino acid sequence of any one of the SEQ ID NOs in Table 10 having a Combined Grade of A/B and/or B/A, optionally wherein the sequence lacks the HA tag sequence (SEQ ID NO: 50). In some embodiments, the dual-specific bifunctional polypeptide comprises or consists of an amino acid sequence of any one of the SEQ ID NOs in Table 11, optionally wherein the western blot analysis depicted in any one of FIG. 12 demonstrate degradation of one or both of the target proteins. In some embodiments, the dual-specific bifunctional polypeptide does not comprise or consist of an amino acid sequence of any one of the SEQ ID NOs in Table 10 having Grades C and/or D for cither target protein, optionally wherein the sequence lacks the HA tag sequence (SEQ ID NO: 50). In some embodiments where the amino acid sequence differs from a recited reference sequence, the difference is the result of the inclusion of one or more modifications relative to the reference sequence. In some embodiments, the one or more modifications comprise a substitution, insertion and/or deletion. In some embodiments, the substitution is a conservative substitution. In some embodiments, the % sequence identity is calculated over the entirety of the reference sequence.

In some embodiments, the dual-specific bifunctional polypeptide targeting MYC comprises embodiments further sharing one or more elements or features. In some embodiments the shared element or feature is the URD name. In some embodiments the shared element or feature is protein from which the URD is derived. In some embodiments the shared element or feature is one or more of the following: the protein from which the URD is derived; the URD name; the type of targeting moiety; the targeting moiety name; the NLS name; the linker name; the position of the URD relative to the targeting moiety (upstream or downstream); and both the URD name and targeting moiety name. In some embodiments, the HA tag sequence (SEQ ID NO: 50) is excluded.

In some embodiments of the dual-specific bifunctional polypeptide targeting MYC the URD is not derived from CHIP. In some embodiments, the URD does not comprise or consist of CHIP.1. In some embodiments, the URD does not comprise or consist of SEQ ID NO: 1836.

In some embodiments, disclosed herein is a polynucleotide encoding any of the dual-specific bifunctional polypeptides targeting MYC disclosed above, or elsewhere herein.

TABLE 9
Exemplary MYC Targeting Moieties

| SEQ ID NO | Category | Name |
|---|---|---|
| 1918 | sdAb full length | mycV.0300 full |
| 1919 | sdAb FR1 | mycV.0300 VH FR1 |
| 1920 | sdAb CDR1 | mycV.0300 VH CDR1 |
| 1921 | sdAb FR2 | mycV.0300 VH FR2 |
| 1922 | sdAb CDR2 | mycV.0300 VH CDR2 |
| 1923 | sdAb FR3 | mycV.0300 VH FR3 |
| 1924 | sdAb CDR3 | mycV.0300 VH CDR3 |
| 1925 | sdAb FR4 | mycV.0300 VH FR4 |
| 1926 | sdAb full length | mycV.1300 full |
| 1927 | sdAb FR1 | mycV.1300 VH FR1 |
| 1928 | sdAb CDR1 | mycV.1300 VH CDR1 |
| 1929 | sdAb FR2 | mycV.1300 VH FR2 |
| 1930 | sdAb CDR2 | mycV.1300 VH CDR2 |
| 1931 | sdAb FR3 | mycV.1300 VH FR3 |
| 1932 | sdAb CDR3 | mycV.1300 VH CDR3 |
| 1933 | sdAb FR4 | mycV.1300 VH FR4 |
| 1934 | sdAb full length | mycV.1500 full |
| 1935 | sdAb FR1 | mycV.1500 VH FR1 |
| 1936 | sdAb CDR1 | mycV.1500 VH CDR1 |
| 1937 | sdAb FR2 | mycV.1500 VH FR2 |
| 1938 | sdAb CDR2 | mycV.1500 VH CDR2 |
| 1939 | sdAb FR3 | mycV.1500 VH FR3 |
| 1940 | sdAb CDR3 | mycV.1500 VH CDR3 |
| 1941 | sdAb FR4 | mycV.1500 VH FR4 |
| 1942 | sdAb full length | mycV.3700 full |
| 1943 | sdAb FR1 | mycV.3700 VH FR1 |
| 1944 | sdAb CDR1 | mycV.3700 VH CDR1 |
| 1945 | sdAb FR2 | mycV.3700 VH FR2 |
| 1946 | sdAb CDR2 | mycV.3700 VH CDR2 |
| 1947 | sdAb FR3 | mycV.3700 VH FR3 |
| 1948 | sdAb CDR3 | mycV.3700 VH CDR3 |
| 1949 | sdAb FR4 | mycV.3700 VH FR4 |
| 1950 | sdAb full length | mycV.6600 full |
| 1951 | sdAb FR1 | mycV.6600 VH FR1 |
| 1952 | sdAb CDR1 | mycV.6600 VH CDR1 |
| 1953 | sdAb FR2 | mycV.6600 VH FR2 |
| 1954 | sdAb CDR2 | mycV.6600 VH CDR2 |
| 1955 | sdAb FR3 | mycV.6600 VH FR3 |
| 1956 | sdAb CDR3 | mycV.6600 VH CDR3 |
| 1957 | sdAb FR4 | mycV.6600 VH FR4 |
| 1958 | sdAb full length | mycV.1516 full |
| 1959 | sdAb FR1 | mycV.1516 VH FR1 |
| 1960 | sdAb CDR1 | mycV.1516 VH CDR1 |
| 1961 | sdAb FR2 | mycV.1516 VH FR2 |
| 1962 | sdAb CDR2 | mycV.1516 VH CDR2 |
| 1963 | sdAb FR3 | mycV.1516 VH FR3 |
| 1964 | sdAb CDR3 | mycV.1516 VH CDR3 |
| 1965 | sdAb FR4 | mycV.1516 VH FR4 |
| 1966 | sdAb full length | mycV.1515 full |
| 1967 | sdAb FR1 | mycV.1515 VH FR1 |
| 1968 | sdAb CDR1 | mycV.1515 VH CDR1 |
| 1969 | sdAb FR2 | mycV.1515 VH FR2 |
| 1970 | sdAb CDR2 | mycV.1515 VH CDR2 |
| 1971 | sdAb FR3 | mycV.1515 VH FR3 |
| 1972 | sdAb CDR3 | mycV.1515 VH CDR3 |
| 1973 | sdAb FR4 | mycV.1515 VH FR4 |
| 1974 | sdAb full length | mycV.1514 full |
| 1975 | sdAb FR1 | mycV.1514 VH FR1 |
| 1976 | sdAb CDR1 | mycV.1514 VH CDR1 |
| 1977 | sdAb FR2 | mycV.1514 VH FR2 |
| 1978 | sdAb CDR2 | mycV.1514 VH CDR2 |
| 1979 | sdAb FR3 | mycV.1514 VH FR3 |
| 1980 | sdAb CDR3 | mycV.1514 VH CDR3 |
| 1981 | sdAb FR4 | mycV.1514 VH FR4 |
| 1982 | sdAb full length | mycV.1513 full |
| 1983 | sdAb FR1 | mycV.1513 VH FR1 |
| 1984 | sdAb CDR1 | mycV.1513 VH CDR1 |
| 1985 | sdAb FR2 | mycV.1513 VH FR2 |
| 1986 | sdAb CDR2 | mycV.1513 VH CDR2 |
| 1987 | sdAb FR3 | mycV.1513 VH FR3 |
| 1988 | sdAb CDR3 | mycV.1513 VH CDR3 |
| 1989 | sdAb FR4 | mycV.1513 VH FR4 |
| 1990 | sdAb full length | mycV.1512 full |
| 1991 | sdAb FR1 | mycV.1512 VH FR1 |
| 1992 | sdAb CDR1 | mycV.1512 VH CDR1 |
| 1993 | sdAb FR2 | mycV.1512 VH FR2 |
| 1994 | sdAb CDR2 | mycV.1512 VH CDR2 |
| 1995 | sdAb FR3 | mycV.1512 VH FR3 |
| 1996 | sdAb CDR3 | mycV.1512 VH CDR3 |
| 1997 | sdAb FR4 | mycV.1512 VH FR4 |
| 1998 | sdAb full length | mycV.1511 full |
| 1999 | sdAb FR1 | mycV.1511 VH FR1 |
| 2000 | sdAb CDR1 | mycV.1511 VH CDR1 |
| 2001 | sdAb FR2 | mycV.1511 VH FR2 |
| 2002 | sdAb CDR2 | mycV.1511 VH CDR2 |
| 2003 | sdAb FR3 | mycV.1511 VH FR3 |
| 2004 | sdAb CDR3 | mycV.1511 VH CDR3 |
| 2005 | sdAb FR4 | mycV.1511 VH FR4 |
| 2006 | sdAb full length | mycV.1510 full |
| 2007 | sdAb FR1 | mycV.1510 VH FR1 |
| 2008 | sdAb CDR1 | mycV.1510 VH CDR1 |
| 2009 | sdAb FR2 | mycV.1510 VH FR2 |
| 2010 | sdAb CDR2 | mycV.1510 VH CDR2 |
| 2011 | sdAb FR3 | mycV.1510 VH FR3 |
| 2012 | sdAb CDR3 | mycV.1510 VH CDR3 |
| 2013 | sdAb FR4 | mycV.1510 VH FR4 |
| 2014 | sdAb full length | mycV.1509 full |
| 2015 | sdAb FR1 | mycV.1509 VH FR1 |
| 2016 | sdAb CDR1 | mycV.1509 VH CDR1 |
| 2017 | sdAb FR2 | mycV.1509 VH FR2 |
| 2018 | sdAb CDR2 | mycV.1509 VH CDR2 |
| 2019 | sdAb FR3 | mycV.1509 VH FR3 |
| 2020 | sdAb CDR3 | mycV.1509 VH CDR3 |
| 2021 | sdAb FR4 | mycV.1509 VH FR4 |
| 2022 | sdAb full length | mycV.1508 full |
| 2023 | sdAb FR1 | mycV.1508 VH FR1 |
| 2024 | sdAb CDR1 | mycV.1508 VH CDR1 |
| 2025 | sdAb FR2 | mycV.1508 VH FR2 |
| 2026 | sdAb CDR2 | mycV.1508 VH CDR2 |
| 2027 | sdAb FR3 | mycV.1508 VH FR3 |
| 2028 | sdAb CDR3 | mycV.1508 VH CDR3 |
| 2029 | sdAb FR4 | mycV.1508 VH FR4 |
| 2030 | sdAb full length | mycV.1507 full |
| 2031 | sdAb FR1 | mycV.1507 VH FR1 |
| 2032 | sdAb CDR1 | mycV.1507 VH CDR1 |
| 2033 | sdAb FR2 | mycV.1507 VH FR2 |
| 2034 | sdAb CDR2 | mycV.1507 VH CDR2 |
| 2035 | sdAb FR3 | mycV.1507 VH FR3 |
| 2036 | sdAb CDR3 | mycV.1507 VH CDR3 |
| 2037 | sdAb FR4 | mycV.1507 VH FR4 |
| 2038 | sdAb full length | mycV.1506 full |
| 2039 | sdAb FR1 | mycV.1506 VH FR1 |
| 2040 | sdAb CDR1 | mycV.1506 VH CDR1 |
| 2041 | sdAb FR2 | mycV.1506 VH FR2 |
| 2042 | sdAb CDR2 | mycV.1506 VH CDR2 |
| 2043 | sdAb FR3 | mycV.1506 VH FR3 |
| 2044 | sdAb CDR3 | mycV.1506 VH CDR3 |
| 2045 | sdAb FR4 | mycV.1506 VH FR4 |
| 2046 | sdAb full length | mycV.1505 full |
| 2047 | sdAb FR1 | mycV.1505 VH FR1 |
| 2048 | sdAb CDR1 | mycV.1505 VH CDR1 |
| 2049 | sdAb FR2 | mycV.1505 VH FR2 |
| 2050 | sdAb CDR2 | mycV.1505 VH CDR2 |
| 2051 | sdAb FR3 | mycV.1505 VH FR3 |
| 2052 | sdAb CDR3 | mycV.1505 VH CDR3 |
| 2053 | sdAb FR4 | mycV.1505 VH FR4 |
| 2054 | sdAb full length | mycV.1504 full |
| 2055 | sdAb FR1 | mycV.1504 VH FR1 |
| 2056 | sdAb CDR1 | mycV.1504 VH CDR1 |
| 2057 | sdAb FR2 | mycV.1504 VH FR2 |
| 2058 | sdAb CDR2 | mycV.1504 VH CDR2 |
| 2059 | sdAb FR3 | mycV.1504 VH FR3 |
| 2060 | sdAb CDR3 | mycV.1504 VH CDR3 |
| 2061 | sdAb FR4 | mycV.1504 VH FR4 |
| 2062 | sdAb full length | mycV.1503 full |
| 2063 | sdAb FR1 | mycV.1503 VH FR1 |
| 2064 | sdAb CDR1 | mycV.1503 VH CDR1 |
| 2065 | sdAb FR2 | mycV.1503 VH FR2 |
| 2066 | sdAb CDR2 | mycV.1503 VH CDR2 |
| 2067 | sdAb FR3 | mycV.1503 VH FR3 |
| 2068 | sdAb CDR3 | mycV.1503 VH CDR3 |
| 2069 | sdAb FR4 | mycV.1503 VH FR4 |
| 2070 | sdAb full length | mycV.1502 full |
| 2071 | sdAb FR1 | mycV.1502 VH FR1 |
| 2072 | sdAb CDR1 | mycV.1502 VH CDR1 |
| 2073 | sdAb FR2 | mycV.1502 VH FR2 |
| 2074 | sdAb CDR2 | mycV.1502 VH CDR2 |
| 2075 | sdAb FR3 | mycV.1502 VH FR3 |
| 2076 | sdAb CDR3 | mycV.1502 VH CDR3 |
| 2077 | sdAb FR4 | mycV.1502 VH FR4 |
| 2078 | sdAb full length | mycV.1501 full |
| 2079 | sdAb FR1 | mycV.1501 VH FR1 |
| 2080 | sdAb CDR1 | mycV.1501 VH CDR1 |
| 2081 | sdAb FR2 | mycV.1501 VH FR2 |
| 2082 | sdAb CDR2 | mycV.1501 VH CDR2 |
| 2083 | sdAb FR3 | mycV.1501 VH FR3 |
| 2084 | sdAb CDR3 | mycV.1501 VH CDR3 |
| 2085 | sdAb FR4 | mycV.1501 VH FR4 |
| 2086 | peptide | OMOMYC |

TABLE 10
Exemplary dual-specific bifunctional polypeptides targeting MYC and
corresponding exemplary target protein degradation grade

| | | | | | SEQ |
|---|---|---|---|---|---|
| | | MYC | CTNNB1 | Combined | ID |
| Targets | Description | Grade | Grade | Grade | NO |
| MYC | mycNLS-mycV.1500-RNF165.1- | A | B | A/B | 2188 |
| CTNNB1 | (GS)₂-TCF4.6 | | | | |
| MYC | mycNLS-mycV.1500-RNF165.1- | A | n.a. | A | 2282 |
| MYC | (GS)₃-mycV.1500 | | | | |
| MYC | spNLS.2-mycV.1500-(GS)₂- | A | n.a. | A | 2283 |
| MYC | RNF4.1-(GS)₂-mycV.1500 | | | | |
| MYC | HA-mycNLS-mycV.1500- | B | A | B/A | 2180 |
| CTNNB1 | GGGS-TCF4.2-GGGS- | | | | |
| | RNF125.2-hrpNLS | | | | |
| MYC | HA-mycNLS-mycV.1500- | B | A | B/A | 2184 |
| CTNNB1 | GGGS-TCF4.2-GGGS- | | | | |
| | NHLRC1.1-hrpNLS | | | | |
| MYC | HA-mycNLS-RNF125.2-GGGS- | C | A | C/A | 2178 |
| CTNNB1 | TCF4.2-GGGS-mycV.1500- | | | | |
| | hrpNLS | | | | |
| MYC | HA-mycNLS-RNF125.2-GGGS- | C | A | C/A | 2179 |
| CTNNB1 | mycV.1500-GGGS-TCF4.2- | | | | |
| | hrpNLS | | | | |
| MYC | HA-mycNLS-NHLRC1.1-GGGS- | C | A | C/A | 2182 |
| CTNNB1 | mycV.1500-GGGS-TCF4.2- | | | | |
| | hrpNLS | | | | |
| MYC | HA-mycNLS-NHLRC1.1-GGGS- | C | A | C/A | 2183 |
| CTNNB1 | TCF4.2-GGGS-mycV.1500- | | | | |
| | hrpNLS | | | | |
| MYC | HA-mycNLS-TCF4.2-GGGS- | C | A | C/A | 2185 |
| CTNNB1 | mycV.1500-GGGS-NHLRC1.1- | | | | |
| | hrpNLS | | | | |
| MYC | HA-mycNLS-mycV.1500- | C | A | C/A | 2186 |
| CTNNBI1 | GGGS-NHLRC1.1-GGGS- | | | | |
| | TCF4.2-hrpNLS | | | | |
| MYC | HA-mycNLS-TCF4.2-GGGS- | D | A | D/A | 2187 |
| CTNNB1 | NHLRC1.1-GGGS-mycV.1500- | | | | |
| | hrpNLS | | | | |
| MYC | HA-mycNLS-TCF4.2-GGGS- | D | B | D/B | 2181 |
| CTNNB1 | mycV.1500-GGGS-RNF125.2- | | | | |
| | hrpNLS | | | | |

TABLE 11
Exemplary dual-specific bifunctional polypeptides targeting MYC with
exemplary western blot data

| Targets | Description | SEQ ID NO |
|---|---|---|
| MYC | HA-mycNLS-TCF4.2-(GS)₃-RNF125.2-(GS)₃-OMOMYC | 2189 |
| CTNNB1 | | |
| MYC | HA-mycNLS-TCF4.3-(GS)₃-RNF125.2-(GS)₃-OMOMYC | 2190 |
| CTNNB1 | | |
| MYC | HA-mycNLS-TCF4.4-(GS)₃-RNF125.2-(GS)₃-OMOMYC | 2191 |
| CTNNB1 | | |
| MYC | HA-mycNLS-TCF4.5-(GS)₃-RNF125.2-(GS)₃-OMOMYC | 2192 |
| CTNNB1 | | |
| MYC | HA-mycNLS-TCF4.6-(GS)₃-RNF125.2-(GS)₃-OMOMYC | 2193 |
| CTNNB1 | | |
| MYC | HA-mycNLS-TCF4.7-(GS)₃-RNF125.2-(GS)₃-OMOMYC | 2194 |
| CTNNB1 | | |
| MYC | HA-mycNLS-TCF4.8-(GS)₃-RNF125.2-(GS)₃-OMOMYC | 2195 |
| CTNNB1 | | |

### Exemplary Dual-Specific Bifunctional Degraders Targeting PCNA

In some embodiments, the target protein is proliferating cell nuclear antigen (PCNA). In some embodiments, the PCNA is a mutant form. An example sequence for PCNA is provided as SEQ ID NO: 2152 (Uniprot #P12004). PCNA is a co-factor for DNA polymerase delta and is involved in DNA synthesis and DNA repair. PCNA is implicated in the pathology of various solid tumor cancers (including but not limited to colorectal cancer, breast cancer, lung cancer, and liver cancer) and hematological malignancies. In some embodiments disclosed herein, the dual-specific bifunctional polypeptide comprises a first and/or second targeting moiety that is capable of binding to PCNA. Some examples of targeting moieties that target PCNA include those listed in Table 12. In some embodiments, other suitable PCNA targeting moieties generally known in the art are used. In some embodiments only one of the two targeting moieties (the first or second; e.g., an N-TBD or a C-TBD) is capable of binding PCNA, In some embodiments, both of the target binding moieties (the first and second; e.g., N-TBD and C-TBD, two N-TBDs, or two C-TBDs) are capable of binding PCNA. The dual-specific bifunctional polypeptides disclosed herein targeting PCNA may be used for the treatment of a cancer, for example, solid tumor cancers (including but not limited to colorectal cancer, breast cancer, lung cancer, and liver cancer) and hematological malignancies.

In some embodiments, the dual-specific bifunctional polypeptide targeting PCNA comprises a localization peptide sequence which directs the dual-specific bifunctional polypeptide to the nucleus of a cell. In some embodiments, the localization peptide sequence comprises or consists of a NLS sequence selected from the group of the NLS sequences of MYC, SPOP, Hrp1, SV40, 53BP1, and nucleoplasmin. In some embodiments, the localization peptide sequence comprises or consists of a NLS sequence selected from the group of the NLS sequence of MYC, optionally mcyNLS and/or 3xmycNLS, SPOP, Hrp1, SV40, optionally svNLS or 3xsvNLS, and nucleoplasmin. In some embodiments, the NLS comprises or consists of a sequence selected from any one of SEQ ID NOs: 51, 1896-1909. In some embodiments, the NLS comprises or consists of a sequence selected from any one of SEQ ID NOs: 1897-1898, and 1900-1904.

In some embodiments, the dual-specific bifunctional polypeptide targeting PCNA comprises a URD derived from a URD selected from the group consisting of CBL-b (Y363E), CHIP, DCAF1, E6AP, FBXW7-alpha, KEAP1, NHLRC1, RNF4, RNF6, RNF12, RNF25, RNF114, RNF125, RNF138, RNF166, SPOP, TRIM21, and VIF. In some embodiments, the URD is a truncation or fragment thereof that retains ubiquitin-proteasome recruiting activity. In some embodiments, the URD is derived from DCAF1, NHLRC1, RNF125, or SPOP. In some embodiments, the URD comprises or consists of DCAF1.1, NHLRC1.1, RNF125.2, RNF125.3, RNF125.4, RNF125.5, RNF125.6, SPOP.2 or SPOP.3. In some embodiments, the URD comprises or consists of optionally NHLRC1.1 or RNF125.2. In some embodiments, the URD is truncation or fragment thereof that retains ubiquitin-proteasome recruiting activity. In some embodiments, the URD comprises or consists of a sequence selected from any one of SEQ ID NOs: 29-43, and 1836-1895. In some embodiments, the URD comprises or consists of a sequence selected from any one of SEQ ID NOs: 1837, 1855, 1856, 1858, and 1870-1874. In some embodiments, the URD comprises or consists of a sequence selected from any one of SEQ ID NOs: 1858 and 1870. In some embodiments, the URD is the URD of CHIP, DCAF1, E6AP, FBXW7a, FBXW7b, beta-TRCP, Keap1, RNF114, RNF125, RNF138, NHLRC1, CBL-b (Y363E), TRIM21, SPOP, or VIF, or a truncation or fragment thereof that retains ubiquitin-proteasome recruiting activity,

In some embodiments, the dual-specific bifunctional polypeptide targeting PCNA comprises a first and/or second targeting moiety that comprises or consists of a targeting moiety disclosed in Table 12. In some embodiments, the first and/or second targeting moiety binds, or binds at least in part, to at least a portion of amino acids 38-48, 123-129, or 251-257 or any combination thereof, of PCNA (SEQ ID NO: 2152). In some embodiments, the first and/or second targeting moiety comprises or consists of an endogenous protein binding partner of the target protein, a fragment and/or a derivative thereof. In some embodiments, the first and/or second targeting moiety comprises or consists of con1, con1.1, con1.2, p21, p21.1, p21.2, p21.3, p21.4, p21.5, p21.6, p21.7, p21.8, p21.9, or a fragment thereof that is capable of binding to the target protein. In some embodiments, the first and/or second targeting moiety does not comprise full length or unmodified con1 or p21. In some embodiments, the first and/or second targeting moiety comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NOs: 2102-2112. In some embodiments where the amino acid sequence differs from a recited reference sequence, the difference is the result of the inclusion of one or more modifications relative to the reference sequence. In some embodiments, the one or more modifications comprise a substitution, insertion and/or deletion. In some embodiments, the substitution is a conservative substitution. In some embodiments, the % sequence identity is calculated over the entirety of the reference sequence.

In some embodiments, the dual-specific bifunctional polypeptide targeting PCNA comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NOs: 2202-2285, and 2284-2286, optionally wherein the sequence lacks the HA tag sequence (SEQ ID NO: 50). In some embodiments, the dual-specific bifunctional polypeptide targeting PCNA comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NOs: 2202-2206, 2217-2220, 2266-2269, and 2271 (SEQ ID NOs in Table 14), optionally wherein the sequence lacks the HA tag sequence (SEQ ID NO: 50). In some embodiments, the dual-specific bifunctional polypeptide comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of the SEQ ID NOs in Table 13, optionally excluding any one of the SEQ ID NOs having a Grade of D degradation of PCNA and/or another target protein, optionally wherein the sequence lacks the HA tag sequence (SEQ ID NO: 50). In some embodiments, the dual-specific bifunctional polypeptide comprises or consists of an amino acid sequence of any one of the SEQ ID NOs in Table 13 having a Grade A degradation of PCNA, and/or a Grade B degradation of PCNA, optionally wherein the sequence lacks the HA tag sequence (SEQ ID NO: 50). In some embodiments, the dual-specific bifunctional polypeptide comprises or consists of an amino acid sequence of any one of the SEQ ID NOs in Table 13 having a Combined Grade of A and/or A/A, optionally wherein the sequence lacks the HA tag sequence (SEQ ID NO: 50). In some embodiments, the dual-specific bifunctional polypeptide comprises or consists of an amino acid sequence of any one of the SEQ ID NOs in Table 13 having a Combined Grade of A/B and/or B/A, optionally wherein the sequence lacks the HA tag sequence (SEQ ID NO: 50). In some embodiments, the dual-specific bifunctional polypeptide comprises or consists of an amino acid sequence of any one of the SEQ ID NOs in Table 14, optionally wherein the western blot analysis depicted in any one of FIG. 14, 15, 17 or 19 demonstrate degradation of one or both of the target proteins. In some embodiments, the dual-specific bifunctional polypeptide does not comprise or consist of an amino acid sequence of any one of the SEQ ID NOs in Table 13 having Grades C and/or D for either target protein, optionally wherein the sequence lacks the HA tag sequence (SEQ ID NO: 50). In some embodiments where the amino acid sequence differs from a recited reference sequence, the difference is the result of the inclusion of one or more modifications relative to the reference sequence. In some embodiments, the one or more modifications comprise a substitution, insertion and/or deletion. In some embodiments, the substitution is a conservative substitution. In some embodiments, the % sequence identity is calculated over the entirety of the reference sequence.

In some embodiments, the dual-specific bifunctional polypeptide targeting PCNA comprises embodiments further sharing one or more elements or features. In some embodiments the shared element or feature is the URD name. In some embodiments the shared element or feature is protein from which the URD is derived. In some embodiments the shared element or feature is one or more of the following: the protein from which the URD is derived; the URD name; the type of targeting moiety; the targeting moiety name; the NLS name; the linker name; the position of the URD relative to the targeting moiety (upstream or downstream); and both the URD name and targeting moiety name. In some embodiments, the HA tag sequence (SEQ ID NO: 50) is excluded.

In some embodiments of the dual-specific bifunctional polypeptide targeting PCNA the URD is not derived from SPOP. In some embodiments, the URD does not comprise or consist of SPOP.1, SPOP.2, or SPOP.3. In some embodiments, the URD does not comprise or consist of a sequence selected from any one of SEQ ID NOs: 1854-1856.

In some embodiments, disclosed herein is a polynucleotide encoding any of the dual-specific bifunctional polypeptides targeting PCNA disclosed above, or elsewhere herein.

TABLE 12
Exemplary PCNA Targeting Moieties

| SEQ ID NO | Category | Name |
|---|---|---|
| 2127 | peptide | con1.1 |
| 2128 | peptide | con1.2 |
| 2129 | peptide | p21.1 |
| 2130 | peptide | p21.2 |
| 2131 | peptide | p21.3 |
| 2132 | peptide | p21.4 |
| 2133 | peptide | p21.5 |
| 2134 | peptide | p21.6 |
| 2135 | peptide | p21.7 |
| 2136 | peptide | p21.8 |
| 2137 | peptide | p21.9 |

TABLE 13
Exemplary dual-specific bifunctional polypeptides targeting PCNA and
corresponding exemplary target protein degradation grade

| | | | | | SEQ |
|---|---|---|---|---|---|
| | | PCNA | CTNNB1 | Combined | ID |
| Targets | Description | Grade | Grade | Grade | NO |
| PCNA | HA-mycNLS-TCF4.1-(GS)₃- | A | A | A/A | 2236 |
| CTNNB1 | NHLRC1.1-(GS)₁-con1.1 | | | | |
| PCNA | HA-mycNLS-TCF4.1-(GS)₃- | A | A | A/A | 2237 |
| CTNNB1 | NHLRC1.1-(GS)₆-con1.1 | | | | |
| PCNA | HA-mycNLS-con1.1-(GS)₁- | A | A | A/A | 2238 |
| CTNNB1 | NHLRC1.1-(GS)₆-TCF4.1 | | | | |
| PCNA | HA-mycNLS-con1.1-(GS)6- | A | A | A/A | 2239 |
| CTNNB1 | NHLRC1.1-(GS)₆-TCF4.1 | | | | |
| PCNA | HA-mycNLS-TCF4.1-con1.1- | A | A | A/A | 2240 |
| CTNNB1 | (GS)₃-NHLRC1.1 | | | | |
| PCNA | HA-mycNLS-TCF4.1-con1.1- | A | A | A/A | 2241 |
| CTNNB1 | (GS)₆-NHLRC1.1 | | | | |
| PCNA | HA-mycNLS-NHLRC1.1-(GS)₆- | A | A | A/A | 2243 |
| CTNNB1 | con1.1-TCF4.1 | | | | |
| PCNA | HA-mycNLS-con1.1-TCF4.1- | A | A | A/A | 2244 |
| CTNNB1 | (GS)₃-NHLRC1.1 | | | | |
| PCNA | HA-mycNLS-NHLRC1.1-(GS)₆- | A | A | A/A | 2245 |
| CTNNB1 | TCF4.1-con1.1 | | | | |
| PCNA | HA-mycNLS-TCF4.1-(GS)₃- | A | — | — | 2203 |
| CTNNB1 | RNF125.2-(GS)₃-con1.1 | | | | |
| PCNA | HA-3xsvNLS-TCF4.1-(GS)₃- | A | — | — | 2273 |
| CTNNB1 | RNF125.2-(GS)₃-p21.1 | | | | |
| PCNA | HA-3xsvNLS-TCF4.1-(GS)₃- | A | — | — | 2274 |
| CTNNB1 | RNF125.2-(GS)₃-p21.2 | | | | |
| PCNA | HA-3xsvNLS-TCF4.1-(GS)₃- | A | — | — | 2275 |
| CTNNB1 | RNF125.2-(GS)₃-p21.3 | | | | |
| PCNA | HA-3xsvNLS-TCF4.1-(GS)₃- | A | — | — | 2277 |
| CTNNB1 | RNF125.2-(GS)₃-p21.5 | | | | |
| PCNA | HA-3xsvNLS-TCF4.1-(GS)₃- | A | — | — | 2279 |
| CTNNB1 | RNF125.2-(GS)₃-p21.7 | | | | |
| PCNA | HA-3xsvNLS-TCF4.1-(GS)₃- | A | — | — | 2280 |
| CTNNB1 | RNF125.2-(GS)₃-p21.8 | | | | |
| PCNA | HA-3xsvNLS-TCF4.1-(GS)₃- | A | — | — | 2281 |
| CTNNB1 | RNF125.2-(GS)₃-p21.9 | | | | |
| PCNA | HA-mycNLS-NHLRC1.1-(GS)₃- | B | A | B/A | 2242 |
| CTNNB1 | con1.1-TCF4.1 | | | | |
| PCNA | HA-3xsvNLS-TCF4.1-(GS)₃- | B | — | — | 2276 |
| CTNNB1 | RNF125.2-(GS)₃-p21.4 | | | | |
| PCNA | HA-3xsvNLS-TCF4.1-(GS)₃- | B | — | — | 2278 |
| CTNNB1 | RNF125.2-(GS)₃-p21.6 | | | | |

TABLE 14
Exemplary dual-specific bifunctional polypeptides targeting PCNA with exemplary
western blot data

| | | SEQ |
|---|---|---|
| | | ID |
| Targets | Description | NO |
| PCNA | con1.1-(GS)₃-RNF125.2-(GS)₃-TCF4.1-mycNLS-HA | 2202 |
| CTNNB1 | | |
| PCNA | HA-mycNLS-TCF4.1-(GS)₃-RNF125.2-(GS)₃-con1.1 | 2203 |
| CTNNB1 | | |
| PCNA | con1.1-SPOP.2-TCF4.1-HA | 2204 |
| CTNNB1 | | |
| PCNA | HA-TCF4.1-SPOP.2-con1.1 | 2205 |
| CTNNB1 | | |
| PCNA | con1.1-(GS)₃-DCAF1.1-(GS)₃-TCF4.1-mycNLS-HA | 2206 |
| CTNNB1 | | |
| PCNA | HA-mycNLS-TCF4.1-(GS)₃-RNF125.2-(GS)₃-con1.1-3xsvNLS | 2217 |
| CTNNB1 | | |
| PCNA | HA-mycNLS-TCF4.1-(GS)₃-RNF125.2-(GS)₃-con1.1-nlpNLS | 2218 |
| CTNNB1 | | |
| PCNA | HA-mycNLS-TCF4.1-(GS)₃-RNF125.2-(GS)₃-con1.1-bpNLS | 2219 |
| CTNNB1 | | |
| PCNA | HA-mycNLS-TCF4.1-(GS)₃-RNF125.2-(GS)₃-con1.1-hrpNLS | 2220 |
| CTNNB1 | | |
| PCNA | HA-mycNLS-con1.1-TCF4.2-(GS)₃-NHLRC1.1 | 2266 |
| CTNNB1 | | |
| PCNA | HA-mycNLS-con1.1-TCF4.12-(GS)₁-NHLRC1.1 | 2267 |
| CTNNB1 | | |
| PCNA | HA-mycNLS-TCF4.12-(GS)₃-RNF125.2-(GS)₃-con1.1-hrpNLS | 2268 |
| CTNNB1 | | |
| PCNA | HA-mycNLS-con1.1-TCF4.2-(GS)₆-NHLRC1.1 | 2269 |
| CTNNB1 | | |
| PCNA | HA-mycNLS-NHLRC1.1-(GS)₁-TCF4.1-con1.1 | 2271 |
| CTNNB1 | | |

### Additional Structural Features of the Polypeptides

The following features are generally applicable to the disclosed herein embodiments of: polypeptides (e.g., URDs) comprising portions derived from a protein selected from the group consisting of a RING family E3 ubiquitin ligase, a cullin family E3 ubiquitin ligase, a homologous to E6AP carboxyl terminus (HECT) family E3 ubiquitin ligase, and a viral homolog of an E3 ubiquitin ligase; dual-specific bifunctional polypeptides; and dual-specific bifunctional polypeptides that promote proteasome-mediated degradation of a target protein(s).

### Linker Peptides

In some embodiments, the polypeptide or the dual-specific bifunctional polypeptide disclosed above and herein further comprise one or more linker peptides. In some embodiments, the linker peptide is a length that allows for ubiquitination of the target protein via the URD when the target protein is bound by the first and/or second targeting moiety. Any suitable linker peptide generally known in the art may be used in embodiments herein. In some embodiments, the linker peptide is positioned between one or more of: the first and the second targeting moieties; the first and/or second targeting moiety and the URD; the NLS and the first and/or second targeting moiety; the NLS and the URD; and/or the NLS and an epitope tag, optionally an HA tag. In some embodiments, the linker peptide is positioned between the first and/or second targeting moiety and the URD. In some embodiments, the linker peptide is 0 (i.e., there is no linker), 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids in length, or a range defined by any two of the preceding values. In some embodiments, the linker peptide is 0-30, 1-30, 2-30, 0-20, 1-20, 2-20, 0-15, 0-12, 0-10, 1-15, 1-12, 1-10, 2-15, 2-12, 2-10, 3-15, 3-12, or 3-10 amino acids in length, optionally 2-30 amino acids in length. In some embodiments, the linker peptide comprises or consists of glycine and serine. In some embodiments, the linker peptide comprises or consists of one or more alpha helixes, optionally flanked by one or more glycine and/or a serine residues. In some embodiments, the linker peptide comprises or consists of the amino acid sequence of any one of SEQ ID NOs: 2113-2151.

Exemplary linkers are embodied in the amino acid sequences of SEQ ID NOs: 44-49 and nucleic acid sequences of SEQ ID NOs: 1180-1185. An exemplary NLS is embodied in the amino acid sequence of SEQ ID NO: 51 and nucleic acid sequence of SEQ ID NO: 1187.

In some embodiments, the dual-specific bifunctional polypeptide further comprises a linker peptide. In some embodiments, the linker peptide is positioned between the first and/or second targeting moiety and the URD. In some embodiments, the linker peptide is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids in length, or any range defined by any two of the aforementioned lengths, for example, 2-40 amino acids, 2-20 amino acids, 2-10 amino acids, 10-20 amino acids, or 20-40 amino acids. In some embodiments, the linker peptide is 2 to 20 amino acids in length. In some embodiments, the linker peptide comprises or consists of glycine and serine. In some embodiments, the linker peptide comprises or consists of the amino acid sequence of any one of SEQ ID NO: 44-49.

TABLE 15
Exemplary linker sequences

| SEQ | Linker | SEQ | Linker |
|---|---|---|---|
| ID NO | Name | ID NO | Name |
| 2113 | 12xGS | 2133 | L1H24L1 |
| 2114 | 9xGS | 2134 | L3H16L3 |
| 2115 | 6xGS | 2135 | L2H16L3 |
| 2116 | 5xGS | 2136 | L1H16L3 |
| 2117 | 4xGS | 2137 | L3H16L2 |
| 2118 | 3xGS | 2138 | L2H16L2 |
| 2119 | 2xGS | 2139 | L1H16L2 |
| 2120 | 1xGS | 2140 | L3H16L1 |
| 2121 | 3xGGGS + GS | 2141 | L2H16L1 |
| 2122 | 3xGGGS | 2142 | L1H16L1 |
| 2123 | 2xGGGS | 2143 | L3H8L3 |
| 2124 | GGGS | 2144 | L2H8L3 |
| 2125 | L3H24L3 | 2145 | LIH8L3 |
| 2126 | L2H24L3 | 2146 | L3H8L2 |
| 2127 | LIH24L3 | 2147 | L2H8L2 |
| 2128 | L3H24L2 | 2148 | L1H8L2 |
| 2129 | L2H24L2 | 2149 | L3H8L1 |
| 2130 | LIH24L2 | 2150 | L2H8L1 |
| 2131 | L3H24L1 | 2151 | L1H8L1 |
| 2132 | L2H24L1 | | |

### Orientation of Elements of Polypeptides

In some embodiments, the polypeptide or the dual-specific bifunctional polypeptide disclosed above and herein comprises a first targeting moiety, a second targeting moiety, and a URD. The orientation of the URD and SRD of exemplary proteins from which URDs can be derived is provided in Table 1, with “URD-SRD” indicating the URD is upstream of the SRD, and “SRD-URD” indicating the SRD is upstream of the URD, when viewed in the standard N- to C-terminal direction. In some embodiments, the orientation of the first and/or second targeting moiety relative to the URD is the same orientation as compared to the naturally occurring substrate recognition domain (SRD) of the protein from which the URD is derived. In some embodiments, the orientation of the first and/or second targeting moiety relative to the URD is the opposite orientation as compared to the naturally occurring substrate recognition domain of the protein from which the URD is derived. In some embodiments, the first and/or second targeting moiety is N-terminal relative to the URD. In some embodiments, the first and/or second targeting moiety is C-terminal relative to the URD. In some embodiments, both the first and the second targeting moiety are N-terminal relative to the URD. In some embodiments, both the first and the second targeting moiety are C-terminal relative to the URD.

### Tag Peptides

In some embodiments, the polypeptide or the dual-specific bifunctional polypeptide disclosed above and herein further comprises an epitope tag. In some embodiments, the epitope tag is a hemagglutinin (HA) tag. In some embodiments, the HA tag comprises or consists of the amino acid sequence of SEQ ID NO: 50. In some embodiments, the polypeptide or dual-specific bifunctional polypeptide does not comprise an epitope tag. In some embodiments, the polypeptide or dual-specific bifunctional polypeptide does not comprise a hemagglutinin (HA) tag. In some embodiments, the polypeptide or dual-specific bifunctional polypeptide does not comprise an HA tag comprising or consisting of the amino acid sequence of SEQ ID NO: 50. In some embodiments, the epitope tag can be used to visualize the dual-specific bifunctional polypeptide. Typically, dual-specific bifunctional polypeptides comprising an epitope tag will be used for experimentation and visualization purposes. In some embodiments, the dual-specific bifunctional polypeptides intended for treatment do not have an epitope tag. In some embodiments, the dual-specific bifunctional polypeptides intended for treatment have an epitope tag.

### Overall Size of the Dual-Specific Bifunctional Polypeptides

In some embodiments, polypeptide, or the dual-specific bifunctional polypeptide is, is about, is at least, is at least about, is not more than, or is not more than about, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950. 1000, 1050, 1100, or 1150 amino acids long, or a range defined by any two of the preceding values, for example, 60-1150 amino acids, 60-800 amino acids, 60-400 amino acids, 400-1150 amino acids, 400-800 amino acids, 600-1150 amino acids, or 100-700 amino acids long, optionally 120-400 or 130-370 amino acids long.

### Binding Characteristics

In some embodiments, the first and/or second targeting moiety and/or dual-specific bifunctional polypeptide of the present disclosure have an equilibrium dissociation constant of less (that is superior binding) than about 10⁻⁷ or 10⁻⁸ M, for example, less than about 10⁻⁹ M or 10⁻¹⁰ M, in some embodiments, less than about 10⁻¹¹ M, 10⁻¹² M, or 10⁻¹³ M. In some embodiments, the first and/or second targeting moiety binds selectively to the target protein. In some embodiments, the first and/or second targeting moiety binds specifically to the target protein.

### Exemplary Polypeptides Defined by Sequence

Disclosed herein are embodiments of polypeptides or dual-specific bifunctional polypeptides, wherein the polypeptide or dual-specific bifunctional polypeptide comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to:
i) any one of SEQ ID NOs: 2172-2207 and 2209-2351,
ii) any one of SEQ ID NOs: 2172-2207 and 2209-2286,
iii) any one of SEQ ID NOs: 2172-2207 and 2209-2351, wherein the sequence lacks the HA tag sequence (SEQ ID NO: 50) or
iv) any one of SEQ ID NOs: 2172-2207 and 2209-2286, wherein the sequence lacks the HA tag sequence (SEQ ID NO: 50).
   In some embodiments, the polypeptide or dual-specific bifunctional polypeptide is a non-natural polypeptide. In some embodiments, the % sequence identity is calculated over the entirety of the reference sequence.

### Modifications

In some embodiments of the polypeptide or dual-specific bifunctional polypeptide disclosed herein wherein the amino acid sequence differs from the recited reference sequence the difference is the result of the inclusion of one or more modifications relative to the reference sequence. In some embodiments, the one or more modifications comprise a substitution, insertion and/or deletion. In some embodiments, the substitution is a conservative substitution. In some embodiments, the % sequence identity is calculated over the entirety of the reference sequence.

### Exemplary Polynucleotides

Also disclosed herein in some embodiments, are polynucleotides encoding for a polypeptide having at least 80%, 85%, 86%, 87%, 88%, 89%, 90%, 91%, 92%, 93%, 94%, 95%, 96%, 97%, 98%, 99%, or 100% sequence identity to any of polypeptides or the dual-specific bifunctional polypeptides disclosed herein. In some embodiments, the polynucleotide is a DNA or an RNA. In some embodiments, the RNA is an mRNA or a circular RNA. In some embodiments, the polynucleotide is packaged in a lipid nanoparticle or a viral vector. In some embodiments, the viral vector is an adenovirus, adeno-associated virus, lentivirus, or retrovirus vector. In some embodiments, the % sequence identity is calculated over the entirety of the reference sequence.

In some embodiments, the DNA the RNA, mRNA or circular RNA comprises one or more modified nucleosides. In some embodiments, up to 100% of the nucleotides comprising uracil are replaced with pseudouridine and/or N1-methylpseudouridine. In some embodiments, the polynucleotide is packaged in a lipid nanoparticle, a polymeric nanoparticle, an extracellular vesicle, optionally an exosome, or a viral vector, optionally a replicating viral vector or a non-replicating viral vector, optionally an adenovirus, adeno-associated virus, lentivirus, or retrovirus vector. Methods of making lipid nanoparticles for RNA delivery are known in the art. In some embodiments, the lipid nanoparticle is made from: an ionizable cationic lipid, a PEGylated lipid, a phospholipid, and cholesterol. In some embodiments, a neutral ionizable amino lipid is used. Lipid nanoparticles for RNA delivery are disclosed in, for example: U.S. Pat. No. 11,338,044B2; US20180153822A1; US20220249699A1; WO2022251953A1; Mitchell et al., “Engineering precision nanoparticles for drug delivery,” Nature Rev. Drug Discovery (2021) 20:101-124; Kulkarni et al., “Lipid Nanoparticles Enabling Gene Therapies: From Concepts to Clinical Utility,” Nucleic Acid Therapeutics (2018) 28(3):146-157; Hou et al. “Lipid nanoparticles for mRNA delivery,” Nature Reviews Materials (2021) 6:1078-1094; each of which is incorporated herein by reference in its entirety.

### Exemplary Pharmaceutical Compositions, Uses, and Methods

Also disclosed herein in some embodiments are pharmaceutical compositions comprising any of the polypeptides, the dual-specific bifunctional polypeptides and/or the polynucleotides disclosed herein and one or more pharmaceutically acceptable excipients, carriers, or diluents. Disclosed herein are pharmaceutical compositions comprising any of the dual-specific bifunctional polypeptides and/or polynucleotides disclosed herein and one or more pharmaceutically acceptable excipients, carriers, or diluents. In some embodiments, the composition is formulated for intravenous, intraperitoneal, intra-arterial, subcutaneous, intramuscular, intrathecal, intratumoral, inhalation, or intracranial administration.

In some embodiments, the polypeptide, dual-specific bifunctional polypeptide, polynucleotide, or pharmaceutical composition disclosed herein is for use in the treatment of a cancer in a patient in need thereof. In some embodiments, the polypeptide, dual-specific bifunctional polypeptide, polynucleotide, or pharmaceutical composition disclosed herein is for use in the manufacture of a medicament. In some embodiments, the medicament is for the treatment of cancer.

Disclosed herein in some embodiments, is a method of treating a subject comprising administering the polypeptide, dual-specific bifunctional polypeptide, polynucleotide, or pharmaceutical composition disclosed herein to a subject in need thereof. In some embodiments, the subject has a cancer.

Disclosed herein in some embodiments, is a method of reducing the amount of a target protein in a cell comprising contacting the cell with the polypeptide, dual-specific bifunctional polypeptide, polynucleotide, or pharmaceutical composition disclosed herein. In some embodiments, the target protein is selected from MYC, (optionally c-MYC), CTNNB1, and/or PCNA. In some embodiments, the cell is in a subject, and the contacting comprises administering the polypeptide, dual-specific bifunctional polypeptide, polynucleotide, or pharmaceutical composition to the subject. In some embodiments, the subject has cancer. In some embodiments, the administering is intravenous, intraperitoneal, intra-arterial, subcutaneous, intramuscular, intrathecal, intra-tumoral, or intracranial administration. In some embodiments, the cell is ex vivo, and said contacting comprises contacting the polypeptide, dual-specific bifunctional polypeptide, polynucleotide, or pharmaceutical composition to the cell ex vivo, optionally in an adoptive cell therapy. In some embodiments, the method further comprises administering the cell to a subject after the contacting step, optionally wherein the subject is also the source of the cell.

In some embodiments, in the polypeptide, dual-specific bifunctional polypeptide, polynucleotide, pharmaceutical composition, or method disclosed herein the polypeptide or dual-specific bifunctional polypeptide promotes proteasome-mediated degradation of the target protein when measured in one or more assays described in and Exemplary Assay for assessing proteasomal-mediated degradation of target protein disclosed herein. In some embodiments, the assay is a HiBiT™ tag assay. In some embodiments, the assay is a Western blot, optionally wherein the proteasome-mediated degradation is inhibited when the cells comprising the target protein and the dual-specific bifunctional polypeptide are treated with a proteasome inhibitor. In some embodiments, the treatment with the proteasome inhibitor reduces the amount of degradation of the target protein by at least 50%, 60%, 70%, 80%, 90%, 95% or 100% as compared to cells not treated with the proteasome inhibitor.

### Exemplary Assays for Assessing Proteasomal-Mediated Degradation of Target Protein(s)

The following is an embodiment of exemplary assays for determining if a dual-specific bifunctional polypeptide promotes proteasomal-mediated degradation of a target protein(s) and therefore that the URD retains ubiquitin-proteasome recruiting activity.

As used herein, a URD is a domain derived from a ubiquitin-proteasome system component that has the physical property of recruiting the ubiquitin-proteasome system machinery and the functional property of enabling the polyubiquitination and degradation of the recruited target(s) of interest.

In an exemplary screening assay, the target(s) of interest (either endogenous and/or exogenous target) is engineered to harbor the HiBiT™ tag (Promega) in a relevant cell line. The cell line is transfected with a plasmid encoding the dual-specific bifunctional polypeptide designed to promote proteasome-mediated degradation of the target(s) of interest. 24 hours post transfection, doxycycline is added to induce expression of the dual-specific bifunctional polypeptide. The HiBiT™ assay is performed the next day according to manufacturer instructions. Loss of HiBiT™ signal is indicative that the dual-specific bifunctional polypeptide promotes proteasome-mediated degradation of the target(s), and that the URD retains ubiquitin-proteasome recruiting activity. A construct that leads to loss of HiBiT™ signal can be subjected to further confirmation in the assay described below. Additional information regarding the HiBiT™ system can be found in Dixon et al. NanoLuc Complementation Reporter Optimized for Accurate Measurement of Protein Interactions in Cells. ACS Chem Biol. (2016) 11(2):400-8, which is hereby expressly incorporated by reference in its entirety.

An exemplary assay for confirmation of proteasome-mediated degradation activity of the dual-specific bifunctional polypeptides is described. This assay can be used in addition to the assay described above, or independent of the above assay. After a suitable time to allow proteasome-mediated degradation of the target(s) of interest (e.g., 24 to 72 hours), lysate of cells expressing the target(s) of interest and the dual-specific bifunctional polypeptide of interest, and control cells that express the target(s) of interest but not the dual-specific bifunctional polypeptide of interest, are subjected to SDS-PAGE, transferred onto a suitable membrane, and probed with antibodies that are specific and selective for the target(s) of interest (Western Blot). Loss of target protein is indicative that the dual-specific bifunctional polypeptide promotes proteasome-mediated degradation of the target, and that the URD retains ubiquitin-proteasome recruiting activity. In some embodiments, to demonstrate that the target loss is proteasome dependent, cells expressing the target and the dual-specific bifunctional polypeptide of interest are treated with a proteasome inhibitor such as bortezomib, MG-132, or lactacystin. The cells expressing the target(s) and the dual-specific bifunctional polypeptide of interest in the presence of the proteasome inhibitor will show a partial (e.g., at least 50%, 60%, 70%, 80%, 90%, or 95%) or total (100%) reduction in amount of degradation as compared to cells not treated with the proteasome inhibitor, which indicates that the loss of the target is proteasome-dependent, which is indicative that the dual-specific bifunctional polypeptide promotes proteasome-mediated degradation of the target(s), and that the URD retains ubiquitin-proteasome recruiting activity. For example, if the cells expressing the target(s) and the dual-specific bifunctional polypeptide of interest in the absence of a proteasome inhibitor reduce the target protein by 90% relative to control cells that express the target(s) of interest but not the dual-specific bifunctional polypeptide of interest, but only by 9% in the presence of a proteasome inhibitor, the proteasome inhibitor reduced the amount of degradation by 90% ((90%−9%)/90%=90%)

Additional target proteins, target binding domains, linker sequences, and other disclosures related to components, features, uses, etc. of bispecific polypeptides are disclosed in PCT/US2023/066619, filed May 4, 2023, which is herein incorporated by reference in its entirety.

### Terms

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, all technical and scientific terms used herein have the same meaning as is commonly understood by one of skill in the art to which the claimed subject matter belongs when read in light of the current disclosure. It is to be understood that the foregoing general description and the following detailed description are exemplary and explanatory only and are not restrictive of any subject matter claimed.

The section headings used herein are for organizational purposes only and are not to be construed as limiting the subject matter described.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that is approximately the recited value, which can vary by as much as 30, 25, 20, 15, 10, 9, 8, 7, 6, 5, 4, 3, 2 or 1% of the recited value. Where it is not clear from the context what is encompassed by “about,” it will mean the value recited +/−10%.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “individual”, “subject”, or “patient” as used herein have their plain and ordinary meaning as understood in light of the specification, and mean a human or a non-human mammal, e.g., a dog, a cat, a mouse, a rat, a cow, a sheep, a pig, a goat, a non-human primate, or a bird, e.g., a chicken, as well as any other vertebrate or invertebrate. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, “in vivo” is given its plain and ordinary meaning as understood in light of the specification and refers to the performance of a method inside living organisms, usually animals, mammals, including humans, and plants, as opposed to a tissue extract or dead organism.

As used herein, “ex vivo” is given its plain and ordinary meaning as understood in light of the specification and refers to the performance of a method outside a living organism with little alteration of natural conditions.

As used herein, “in vitro” is given its plain and ordinary meaning as understood in light of the specification and refers to the performance of a method outside of biological conditions, e.g., in a petri dish or test tube.

The terms “nucleic acid” or “nucleic acid molecule” as used herein have their plain and ordinary meaning as understood in light of the specification, and refer to polynucleotides, such as deoxyribonucleic acid (DNA) or ribonucleic acid (RNA), oligonucleotides, those that appear in a cell naturally, fragments generated by the polymerase chain reaction (PCR), and fragments generated by any of ligation, scission, endonuclease action, and exonuclease action. Nucleic acid molecules can be composed of monomers that are naturally occurring nucleotides (such as DNA and RNA), or analogs of naturally occurring nucleotides (e.g., enantiomeric forms of naturally occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally occurring or modified nucleic acid bases attached to a polyamide backbone. Nucleic acids can be either single stranded or double stranded. “Oligonucleotide” can be used interchangeable with nucleic acid and can refer to either double stranded or single stranded DNA or RNA. A nucleic acid or nucleic acids can be contained in a nucleic acid vector or nucleic acid construct (e.g., plasmid, virus, retrovirus, lentivirus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g. linkers, repeats or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a nucleic acid as used herein has its plain and ordinary meaning as understood in light of the specification and refers to a sequence being after the 3′-end of a previous sequence, on the strand containing the encoding sequence (sense strand) if the nucleic acid is double stranded. The term “upstream” on a nucleic acid as used herein has its plain and ordinary meaning as understood in light of the specification and refers to a sequence being before the 5′-end of a subsequent sequence, on the strand containing the encoding sequence (sense strand) if the nucleic acid is double stranded. The term “grouped” on a nucleic acid as used herein has its plain and ordinary meaning as understood in light of the specification and refers to two or more sequences that occur in proximity either directly or with extra nucleic acids in between, e.g. linkers, repeats, or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths, but generally not with a sequence in between that encodes for a functioning or catalytic polypeptide, protein, or protein domain.

The nucleic acids described herein comprise nucleobases. Primary, canonical, natural, or unmodified bases are adenine, cytosine, guanine, thymine, and uracil. Other nucleobases include but are not limited to purines, pyrimidines, modified nucleobases, 5-methylcytosine, pseudouridine, dihydrouridine, inosine, 7-methylguanosine, hypoxanthine, xanthine, 5,6-dihydrouracil, 5-hydroxymethylcytosine, 5-bromouracil, isoguanine, isocytosine, aminoallyl bases, dye-labeled bases, fluorescent bases, or biotin-labeled bases. In some embodiments, the uridine of the RNA, mRNA or circular RNA is pseudouridine. In some embodiments, the uridine of the RNA, mRNA or circular RNA is N1-methylpseudouridine. In some embodiments, the uracil of the RNA, mRNA or circular RNA is a mixture of pseudouridine and N1-methylpseudouridine. In some embodiments, up to 100% of the nucleotides comprising uracil are replaced with pseudouridine and/or N1-methylpseudouridine.

The terms “peptide”, “polypeptide”, and “protein” as used herein have their plain and ordinary meaning as understood in light of the specification and refer to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein has its plain and ordinary meaning as understood in light of the specification and refers to a sequence being after the C-terminus of a previous sequence. The term “upstream” on a polypeptide as used herein has its plain and ordinary meaning as understood in light of the specification and refers to a sequence being before the N-terminus of a subsequent sequence.

A polypeptide or amino acid sequence “derived from” a designated protein refers to the origin of the polypeptide. Preferably, the polypeptide has an amino acid sequence that is essentially identical to that of a polypeptide encoded in the sequence, or a portion thereof, or which is immunologically identifiable with a polypeptide encoded in the sequence. This terminology also includes a polypeptide expressed from a designated nucleic acid sequence. Peptide sequences having at least 80%, 81%, 82%, 83%, 84%, 85%, 86%, 87%, 88%, 89%, 90%, 91%, 92%, 93%, 94%, 95%, 96%, 97%, 98%, 99%, or 100% homology to any one of the peptide sequences disclosed herein and having the same or similar functional properties are envisioned. The percent homology may be determined according to amino acid substitutions, deletions, or additions between two peptide sequences. Peptide sequences having some percent homology to any one of the peptide sequences disclosed herein may be produced and tested by one skilled in the art through conventional methods.

The term “artificial” or “non-natural” denotes that the polypeptide or portion of a polypeptide in question has a sequence that is not present, in the noted state, in nature. In the present context the polypeptides have been altered from their native state, so that their sequences are no longer those found in wild-type proteins. Where a portion derived from a naturally occurring protein (e.g., a URD or NLS) is a fragment of the naturally occurring protein which does not contain an altered sequence (other than being less than the complete sequence of the naturally occurring protein), a “non-natural” polypeptide comprising the derived portion does not encompass the naturally occurring protein from which the portion is derived. That is to say that a “non-natural” polypeptide comprising the portion must contain additional amino acids which are not found in the natural protein.

As disclosed herein, sequences having a % identity to any of the sequences disclosed herein are envisioned and may be used. The terms “% identity” refer to the percentage of units (i.e., amino acids or nucleotides) that are the same between two or more sequences relative to the length of the sequence. When the two or more sequences being compared are the same length, the % identity will be respective to that length. When two or more sequences being compared are different lengths, deletions and/or insertions may be introduced to obtain the best alignment. In some embodiments, these sequences may include peptide sequences, nucleic acid sequences, CDR sequences, variable region sequences, or heavy or light chain sequences. In some embodiments, any sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any of the sequences disclosed herein may be used. In some embodiments, any sequence having at least 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 substitutions, deletions, or additions relative to any of the sequences disclosed herein may be used. The changes in sequences may apply to, for example, single amino acids, single nucleic acid bases, or nucleic acid codons; however, differences in longer stretches of sequences are also envisioned. As applied to sequences associated with antibodies or binding regions thereof, these differences in sequences may apply to antigen-binding regions (e.g., CDRs) or regions that do not bind to antigens or are only secondary to antigen binding (e.g., framework regions).

As disclosed herein, sequences having a % homology to any of the sequences disclosed herein are envisioned and may be used. The term “% homology” refers to the degree of conservation between two sequences when considering their three-dimensional structure. For example, homology between two protein sequences may be dependent on structural motifs, such as beta strands, alpha helices, and other folds, as well as their distribution throughout the sequence. Homology may be determined through structural determination, either empirically or in silico. In some embodiments, any sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence homology to any of the sequences disclosed herein may be used. In some embodiments, any sequence having at least 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 substitutions, deletions, or additions relative to any of the sequences disclosed herein, which may or may not affect the overall % homology, may be used.

As applied herein, sequences having a certain % similarity to any of the sequence disclosed herein are envisioned and may be used. In some embodiments, these sequences may include peptide sequences, nucleic acid sequences, CDR sequences, variable region sequences, or heavy or light chain sequences. As understood in the art with respect to peptide sequences, “similarity” refers to the comparison of amino acids based on their properties, including but not limited to size, polarity, charge, pK, aromaticity, hydrogen bonding properties, or presence of functional groups (e.g., hydroxyl, thiol, amine, carboxyl, and the like). The term “% similarity” refers to the percentage of units (e.g., amino acids) that are the same between two or more sequences relative to the length of the sequence. When the two or more sequences being compared are the same length, the % similarity will be respective that length. When two or more sequences being compared are different lengths, deletions and/or insertions may be introduced to obtain the best alignment. The similarity of two amino acids may dictate whether a certain substitution is conservative or non-conservative. Methods of determining the conservativeness of an amino acid substitution are generally known in the art and may involve substitution matrices. Commonly used substitution matrices include BLOSUM45, BLOSUM62, BLOSUM80, PAM100, PAM120, PAM160, PAM200, PAM250, but other substitution matrices or approaches may be used as considered appropriate by the skilled person. A certain substitution matrix may be preferential over the others when considering aspects such as stringency, conservation and/or divergence of related sequences (e.g., within the same species or broader), and length of the sequences in question. As used herein, a peptide sequence having a certain % similarity to another sequence will have up to that % of amino acids that are either identical or an acceptable substitution as governed by the method of similarity determination used. In some embodiments, a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to any of the sequences disclosed herein may be used. In some embodiments, any sequence having at least 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 similar substitutions relative to any of the sequences disclosed herein may be used. As applied to sequences associated with antibodies or binding regions thereof, these similar substitutions may apply to antigen-binding regions (i.e., CDRs) or regions that do not bind to antigens or are only secondary to antigen binding (i.e., framework regions). In some embodiments, the percent identity is over the CDR and/or FR regions noted herein. In such situations, the percent identity of the CDR or FR can be identified separately from the rest of the protein or nucleic acid sequence. Thus, two CDRs or FRs can have a specified percentage of amino acid residues or nucleotides that are the same (for example, 70%, 75%, 80%, 85%, 90%, 95%, 96%, 97%, 98% or 99% sequence identity over a specified region, or, when not specified, over the entire sequence of a reference sequence), while allowing for the remainder of the protein to either stay 100% identical to the comparison protein, our while also allowing the remainder of the protein to also have variation by a specified percent identity.

The term “consensus sequence” as used herein with regard to sequences has its plain and ordinary meaning as understood in light of the specification, and refers to the generalized sequence representing all of the different combinations of permissible amino acids at each location of a group of sequences. A consensus sequence may provide insight into the conserved regions of related sequences where the unit (e.g., amino acid or nucleotide) is the same in most or all of the sequences, and regions that exhibit divergence between sequences. In the case of sequences associated with antibodies or binding regions thereof, the consensus sequence of a CDR may indicate amino acids that are important or dispensable for antigen binding. It is envisioned that consensus sequences may be prepared with any of the sequences provided herein, and the resultant various sequences derived from the consensus sequence can be validated to have similar effects as the template sequences.

The term “purity” of any given substance, compound, or material as used herein has its plain and ordinary meaning as understood in light of the specification and refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to nucleic acids, DNA, RNA, nucleotides, proteins, polypeptides, peptides, amino acids, lipids, cell membrane, cell debris, small molecules, degradation products, solvent, carrier, vehicle, or contaminants, or any combination thereof. In some embodiments, the substance, compound, or material is substantially free of host cell proteins, host cell nucleic acids, plasmid DNA, contaminating viruses, proteasomes, host cell culture components, process related components, mycoplasma, pyrogens, bacterial endotoxins, and adventitious agents. Purity can be measured using technologies including but not limited to electrophoresis, SDS-PAGE, capillary electrophoresis, PCR, ItPCR, qPCR, chromatography, liquid chromatography, gas chromatography, thin layer chromatography, enzyme-linked immunosorbent assay (ELISA), spectroscopy, UV-visible spectrometry, infrared spectrometry, mass spectrometry, nuclear magnetic resonance, gravimetry, or titration, or any combination thereof.

The term “yield” of any given substance, compound, or material as used herein has its plain and ordinary meaning as understood in light of the specification and refers to the actual overall amount of the substance, compound, or material relative to the expected overall amount. For example, the yield of the substance, compound, or material is, is about, is at least, is at least about, is not more than, or is not more than about, 80, 85, 90, 91, 92, 93, 94, 95, 96, 97, 98, 99, or 100% of the expected overall amount, including all decimals in between. Yield may be affected by the efficiency of a reaction or process, unwanted side reactions, degradation, quality of the input substances, compounds, or materials, or loss of the desired substance, compound, or material during any step of the production.

The terms “effective amount” or “effective dose” as used herein have their plain and ordinary meaning as understood in light of the specification, and refer to that amount of a recited composition or compound that results in an observable effect. Actual dosage levels of active ingredients in an active composition of the presently disclosed subject matter can be varied so as to administer an amount of the active composition or compound that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

The terms “function” and “functional” as used herein have their plain and ordinary meaning as understood in light of the specification, and refer to a biological, enzymatic, or therapeutic function.

The term “inhibit” as used herein has its plain and ordinary meaning as understood in light of the specification, and may refer to the reduction or prevention of a biological activity. The reduction can be by a percentage that is, is about, is at least, is at least about, is not more than, or is not more than about, 10%, 20%, 30%, 40%, 50%, 60%, 70%, 80%, 90%, or 100%, or an amount that is within a range defined by any two of the aforementioned values. As used herein, the term “delay” has its plain and ordinary meaning as understood in light of the specification, and refers to a slowing, postponement, or deferment of a biological event, to a time which is later than would otherwise be expected. The delay can be a delay of a percentage that is, is about, is at least, is at least about, is not more than, or is not more than about, 0%, 10%, 20%, 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

As used herein, the terms “treating” or “treatment” have their plain and ordinary meaning as understood in light of the specification, and refer to an approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delaying or slowing of disease progression, amelioration or palliation of the disease state, diminishment of the recurrence of disease, and remission, whether partial or total and whether detectable or undetectable. “Treating” and “treatment” as used herein also include prophylactic treatment. Treatment methods comprise administering to a subject a therapeutically effective amount of an active agent. The administering step may consist of a single administration or may comprise a series of administrations. The compositions are administered to the subject in an amount and for a duration sufficient to treat the subject. The length of the treatment period depends on a variety of factors, such as the severity of the condition, the age and genetic profile of the subject, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required.

“Tumor,” as used herein, plain and ordinary meaning as understood in light of the specification, and refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein. The term “neoplasia” encompasses the term tumor,

The terms “cancer” and “cancerous” plain and ordinary meaning as understood in light of the specification, and refer to or describe the physiological condition in mammals that is typically characterized by unregulated cell growth. Examples of cancer include, but are not limited to, carcinoma, lymphoma, blastoma, sarcoma, and leukemia or lymphoid malignancies. More particular examples of such cancers include lung cancer including small-cell lung cancer, non-small cell lung cancer and lung adenocarcinomas with neuroendocrine features; neuroendocrine prostate cancer, melanoma, gliomas, low-grade gliomas and glioblastoma, medullary thyroid cancer, carcinoid tumors, neuroendocrine tumors in the pancreas, bladder cancer, testicular cancer squamous cell cancer (e.g. epithelial squamous cell cancer), neuroendocrine neoplasms, such as neuroendocrine tumors of unknown primary, neuroendocrine neoplasms of the small bowel, carotid body, adrenal gland, colorectal gynecological organ, abdomen, esophagus, GI tract, bile duct, nervous system, appendix, liver, anal, thymus, ileocecal junction, head and neck, breast, peritoneum and retroperitoneum, kidney, thyroid, stomach, bone, adenocarcinomas, such as adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, bone cancer, hepatic carcinoma, anal carcinoma, penile carcinoma, melanoma, multiple myeloma and B-cell lymphoma, brain, as well as head and neck cancer, and associated metastases. The term cancer includes adult and pediatric solid cancers. In some embodiments, the cancer can be a solid tumor. In some embodiments, the cancer is a highly fibrotic tumor or cancer. In some embodiments, the cancer is a desmoplasia.

As used herein, the term “therapeutic target” refers to a gene or gene product that, upon modulation of its activity (e.g., by modulation of expression, biological activity, and the like), can provide for modulation of the disease phenotype. As used throughout, “modulation” is meant to refer to an increase or a decrease in the indicated phenomenon (e.g., modulation of a biological activity refers to an increase in a biological activity or a decrease in a biological activity).

The term “administering” includes oral administration, topical contact, administration as a suppository, intravenous, intraperitoneal, intramuscular, intralesional, intra-tumoral, intrathecal, intranasal, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intra-tumoral, intraventricular, and intracranial. Other modes of delivery include, but are not limited to, the use of liposomal formulations, intravenous infusion, transdermal patches, etc. By “co-administer” it is meant that a first compound described herein is administered at the same time, just prior to, or just after the administration of a second compound described herein.

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or carrier can refer to a diluent, adjuvant, excipient, or vehicle with which the pharmaceutical formulation is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include sugars, starch, glucose, fructose, lactose, sucrose, maltose, gelatin, malt, rice, flour, chalk, silica gel, sodium stearate, glycerol monostearate, talc, salts, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as glycerol, erythritol, threitol, arabitol, xylitol, ribitol, mannitol, sorbitol, galactitol, fucitol, iditol, inositol, isomalt, maltitol, or lactitol, salt-forming counterions such as sodium, and nonionic surfactants such as TWEEN®, polyethylene glycol (PEG), and PLURONICS®. The formulation, if desired, can also contain minor amounts of wetting, bulking, emulsifying agents, or pH buffering agents. These formulations can take the form of solutions, suspensions, emulsion, sustained release formulations and the like. The formulation should suit the mode of administration.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, mannitol, cellulose, serum, amino acids, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, polysorbate 20, polysorbate 40, polysorbate, 60, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, β-propiolactone, gelatin, cell debris, nucleic acids, peptides, amino acids, or growth medium components or any combination thereof. The amount of the excipient may be found in the formulation at a percentage that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphencthylamine; trihydroxymethyl aminocthane.

The term “ubiquitin-proteasome recruiting activity” as used herein has its plain and ordinary meaning as understood in light of the specification and refers to the biological function of binding with proteins involved in the ubiquitination pathway to affect the ubiquitination of a target protein. More specifically in the embodiments disclosed herein, this generally applies to the ability of a peptide to recruit a ubiquitinated E2 ubiquitin-conjugating enzyme and catalyze the transfer of the ubiquitin from the E2 ubiquitin-conjugate enzyme to the target protein. The peptide having this ubiquitin-proteasome recruiting activity will therefore generally be a “ubiquitin-proteasome system recruiting domain (URD)”, where the URD has the ability to recruit a ubiquitin E2 ubiquitin-conjugating enzyme and catalyze the transfer of the ubiquitin from the E2 ubiquitin-conjugate enzyme to the target protein. In some embodiments provided herein, the URD is a portion, truncation, or fragment of a naturally occurring E3 ubiquitin ligase, where the portion, truncation, or fragment of the E3 ubiquitin ligase exhibits the ubiquitin-proteasome recruiting activity. The E3 ubiquitin ligase can be obtained from any source (e.g., from any eukaryote or archaea) but, in some embodiments, is preferably a human E3 ubiquitin ligase. Nevertheless, the URD may also be derived from a protein that is not a E3 ubiquitin ligase but has a similar function, such as a viral analogue of an E3 ubiquitin ligase (e.g., VIF from HIV). Different portions, truncations, or fragments of an E3 ubiquitin ligase or analogue may exhibit a ubiquitin-proteasome recruiting activity, and it would be within the capabilities of a skilled person to determine a certain portion, truncation, or fragment of an E3 ubiquitin ligase or analogue that is preferable for their uses (e.g., exhibiting better E2 ubiquitin-conjugating enzyme binding activity, reducing off-target or off-mechanism protein-protein interactions, and/or more efficiently catalyzing ubiquitination of a target protein). It is envisioned that the URDs or the portions, truncations, or fragments of the URDs and/or E3 ubiquitin ligases disclosed herein can be reasonably modified while still retaining their ubiquitin-proteasome recruiting activity, for example, by including or excluding amino acids from the N-terminus or C-terminus of the URD or the portion, truncation, or fragment of the URD and/or E3 ubiquitin ligase or performing amino acid substitutions to enhance various aspects of the URD and/or the portion, truncation, or fragment of the URD and/or E3 ubiquitin ligase such as protein folding, expression, or catalytic activity, optionally within the context of the dual-specific bifunctional polypeptide.

The ubiquitin-proteasome recruiting activity of a certain URD and/or portion, truncation, or fragment of a URD and/or E3 ubiquitin ligase may be quantified or verified through methods generally known in the art. For example, activity may be observed by observing (a) loss of bioluminescent signal from a HiBiT™-tagged intracellular target protein, (b) loss of target protein signature and/or presence of polyubiquitin chain in target protein by mass spectrometry, (c) ubiquitination of the target protein in cell or in recombinant in vitro assays, or (d) detecting ubiquitination of target protein by immunoprecipitation, upon introduction of a URD-containing polypeptide specific for a known target protein. These exemplary approaches are explored in Franklin et al. A High-Throughput Assay for Monitoring Ubiquitination in Real Time. Front. Chem. (2019) 7:816, Choo & Zhang. Detection of protein ubiquitination. J Vis. Exp. (2009) 30:1293, Udeshi et al. Large-scale identification of ubiquitination sites by mass spectrometry. Nat Protoc. (2013) 8(10):1950-60, and Riching et al. Quantitative Live-Cell Kinetic Degradation and Mechanistic Profiling of PROTAC Mode of Action. ACS Chem Biol. (2018) 13(9):2758-2770, each of which are expressly incorporated by reference in its entirety. In some embodiments, a URD that retains ubiquitin-proteasome recruiting activity has polyubiquitination activity against a target bound by its targeting moiety when measured in an assay disclosed in one of the preceding references. In some embodiments, a URD that retains ubiquitin-proteasome recruiting activity has polyubiquitination activity against a target bound by its targeting moiety when measured in an assay described in Exemplary Assays for assessing proteasomal-mediated degradation and/or Example 1. In some embodiments, a URD that retains ubiquitin-proteasome recruiting activity has polyubiquitination activity against a target bound by its targeting moiety when measured by Western blot (e.g., measuring the abundance of the target in cells). In some embodiments, a URD that retains ubiquitin-proteasome recruiting activity has polyubiquitination activity against a target bound by its targeting moiety when measured by Western blot, but where the target is partially or totally preserved when the cells comprising the URD and target are treated with a proteasome inhibitor.

The term “antibody” includes, but is not limited to, genetically engineered or otherwise modified forms of immunoglobulins, such as intrabodies, chimeric antibodies, fully human antibodies, humanized antibodies, antibody fragments, scFv, and heteroconjugate antibodies (for example, bispecific antibodies, diabodies, triabodies, tetrabodies, and nanobodies, etc.). The term “antibody” includes minibodies and diabodies. The term “antibody” includes a polypeptide of the immunoglobulin family or a polypeptide comprising fragments of an immunoglobulin that is capable of noncovalently, reversibly, and in a specific manner binding a corresponding antigen. An exemplary antibody structural unit comprises a tetramer. In some embodiments, a full length antibody can be composed of two identical pairs of polypeptide chains, each pair having one “light” and one “heavy” chain (connected through a disulfide bond). The recognized immunoglobulin genes include the kappa, lambda, alpha, gamma, delta, epsilon, hinge, and mu constant region genes, as well as the myriad immunoglobulin variable region genes. For full length chains, the light chains are classified as either kappa or lambda. For full length chains, the heavy chains are classified as gamma, mu, alpha, delta, or epsilon, which in turn define the immunoglobulin classes, IgG, IgM, IgA, IgD, and IgE, respectively. The N-terminus of each chain defines a variable region of about 100 to 110 or more amino acids primarily responsible for antigen recognition. The terms variable light chain (V_{L}) and variable heavy chain (V_{H}) refer to these regions of light and heavy chains respectively. As used in this application, an “antibody” encompasses all variations of antibody and fragments thereof. Thus, within the scope of this concept are full length antibodies, chimeric antibodies, humanized antibodies, single chain antibodies (scFv), Fab, Fab′, and multimeric versions of these fragments (for example, F(ab′)₂) with the same binding specificity. In some embodiments, the antibody binds specifically to a desired target.

The term “complementarity-determining domains” or “complementarity-determining regions (“CDRs”) interchangeably refer to the hypervariable regions of V_{L} and V_{H}. The CDRs are the target molecule-binding site of the antibody chains that harbors specificity for such target molecule. In some embodiments, there are three CDRs (CDR1-3, numbered sequentially from the N-terminus) in each V_{L} and/or V_{H}, constituting about 15-20% of the variable domains. The CDRs are structurally complementary to the epitope of the target molecule and are thus directly responsible for the binding specificity. The remaining stretches of the V_{L} or V_{H}, the so-called FRs, exhibit less variation in amino acid sequence (Kuby, Immunology, 4th ed., Chapter 4. W.H. Freeman & Co., New York, 2000).

The positions of the CDRs and framework regions can be determined using various well known definitions in the art. In some embodiments, the polypeptide is numbered from the beginning of the polypeptide signal sequence. In some embodiments, the polypeptide is numbered according from the beginning of the polypeptide and not including the signal sequence.

An “antibody variable light chain” or an “antibody variable heavy chain” as used herein have their plain and ordinary meaning as understood in light of the specification, and refers to a polypeptide comprising the V_{L} or V_{H}, respectively. The endogenous V_{L} is encoded by the gene segments V (variable) and J (junctional), and the endogenous V_{H} by V, D (diversity), and J. Each of V_{L} or V_{H} includes the CDRs as well as the framework regions. In this application, antibody variable light chains and/or antibody variable heavy chains may, from time to time, be collectively referred to as “antibody chains.” These terms encompass antibody chains containing mutations that do not disrupt the basic structure of V_{L} or V_{H}, as one skilled in the art will readily recognize. In some embodiments, full length heavy and/or light chains are contemplated. In some embodiments, only the variable region of the heavy and/or light chains are contemplated as being present.

Antibodies can exist as intact immunoglobulins or as a number of fragments produced by digestion with various peptidases as is known in the art. While various antibody fragments are defined in terms of the digestion of an intact antibody, one of skill will appreciate that such fragments may be synthesized de novo cither chemically or by using recombinant DNA methodology. Thus, the term “antibody,” as used herein, has its plain and ordinary meaning as understood in light of the specification, and also includes antibody fragments either produced by the modification of whole antibodies, or those synthesized de novo using recombinant DNA methodologies (for example, single chain Fv) or those identified using phage display libraries (see, for example, McCafferty, J. et al., “Phage antibodies: filamentous phage displaying antibody variable domains,” Nature, Vol. 348, No. 66301, pp. 552-554, 1990). For preparation of monoclonal or polyclonal antibodies, any technique known in the art can be used. Techniques for the production of single chain antibodies (U.S. Pat. No. 4,946,778) can be adapted to produce antibodies to polypeptides of the present disclosure. Also, transgenic mice, or other organisms such as other mammals, may be used to express fully human monoclonal antibodies. Furthermore, E. coli or yeast may be used to express and manufacture recombinant antibodies and antibody fragments. Alternatively, phage display and yeast display technologies can be used to identify high affinity binders to selected antigens. Alternatively, antibodies can be produced through B-cell screening technologies from human hosts (Pedrioli A., Oxenius A., Single B cell technologies for monoclonal antibody discovery, Trends in Immunology, (2021) volume 42, issue 12, p1143-1158). Furthermore, antibodies can be derived from immunization of camelid animals or screening of camelid phage libraries.

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. In some embodiments, the terms “donor” and “acceptor” sequences can be employed. Humanization can be essentially performed following the method of Winter and co-workers by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some complementarity determining region (“CDR”) residues and possibly some framework (“FR”) residues are substituted by residues from analogous sites in rodent antibodies.

The term “antibody fragment” includes but is not limited to one or more antigen binding fragments of antibodies alone or in combination with other molecules, including, but not limited to Fab′, F(ab′)₂, Fab, Fv, rIgG (reduced IgG), scFv fragments, single domain fragments (nanobodies), peptibodies, nanobodies), minibodies, and diabodies. The term “scFv” refers to a single chain Fv (“fragment variable”) antibody in which the variable domains of the heavy chain and of the light chain of a traditional two chain antibody have been joined to form one chain.

A “diabody” comprises a first polypeptide chain which comprises a heavy (VH) chain variable domain connected to a light chain variable domain (VL) on the first polypeptide chain (VH-VL) connected by a peptide linker that is too short to allow pairing between the two domains on the first polypeptide chain and a second polypeptide chain comprising a light chain variable domain (VL) linked to a heavy chain variable domain VH on the second polypeptide chain (VL-VH) connected by a peptide linker that is too short to allow pairing between the two domains on the second polypeptide chain. The short linkages force chain pairing between the complementary domains of the first and the second polypeptide chains and promotes the assembly of a dimeric molecule with two functional antigen binding sites. Therefore, a peptide linker may be any suitable length that promotes such assembly, for example, between 5 and 20 amino acids in length. A “cys-diabody” is a diabody with one or more than one N-terminal cysteines.

In some embodiments, the targeting moiety includes all varieties of antibodies, including binding fragments thereof. Further included are constructs that include 1, 2, 3, 4, 5, and/or 6 CDRs. In some embodiments, these CDRs can be distributed between their appropriate framework regions in a traditional antibody. In some embodiments, the CDRs can be contained within a heavy and/or light chain variable region. In some embodiments, the CDRs can be within a heavy chain and/or a light chain. In some embodiments, the CDRs can be within a single peptide chain.

The terms “specific” “specifically” “selective” “selectively” and the like when used in the context of describing the interaction between a target protein and a targeting moiety, refers to a binding reaction that is determinative of the presence of the target protein in a heterogeneous population of proteins and other biologics, for example, in a biological sample, for example, a blood, serum, plasma or tissue sample. Thus, under designated immunoassay conditions, in some embodiments, the targeting moieties (e.g., protein binding partners, antibodies) with a particular binding specificity bind to a particular target protein at least two times the background and do not substantially bind in a significant amount to other non-target molecules present in the sample. Specific binding to a targeting moiety under such conditions may require the targeting moiety to have been selected for its specificity for a particular target protein. A variety of assay formats may be used to select targeting moieties specifically reactive with a particular target protein. For example, solid-phase ELISA immunoassays are routinely used to select antibodies specifically immunoreactive with a protein (see, for example, Harlow, E. & Lane D., “Using Antibodies, A Laboratory Manual,” Cold Spring Harbor Laboratory Press, 1998, for a description of immunoassay formats and conditions that can be used to determine specific immunoreactivity). Typically, a specific or selective binding reaction will produce a signal at least twice over the background signal and more typically at least than 10 to 100 times over the background.

The term “equilibrium dissociation constant (K_{D}, M)” refers to the dissociation rate constant (k_{d}, time⁻¹) divided by the association rate constant (kₐ, time⁻¹ M⁻¹). Equilibrium dissociation constants can be measured using any known method in the art. In some embodiments, the targeting moieties and/or bifunctional polypeptide of the present disclosure generally will have an equilibrium dissociation constant of less (that is superior binding) than about 10⁻⁷ or 10⁻⁸ M, for example, less than about 10⁻⁹ M or 10⁻¹⁰ M, in some embodiments, less than about 10⁻¹¹ M, 10⁻¹² M, or 10⁻¹³ M.

The term “conservative” modification, substitution, or variant applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GOG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence. As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservative” modification, substitution or variant where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the present disclosure.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, for example, Creighton, T. E., “Proteins-Structures and Molecular Properties,” W. H. Freeman & Co. Ltd., 1984).

The term “% w/w” or “% wt/wt” as used herein has its plain and ordinary meaning as understood in light of the specification and refers to a percentage expressed in terms of the weight of the ingredient or agent over the total weight of the composition multiplied by 100. The term “% v/v” or “% vol/vol” as used herein has its plain and ordinary meaning as understood in the light of the specification and refers to a percentage expressed in terms of the liquid volume of the compound, substance, ingredient, or agent over the total liquid volume of the composition multiplied by 100.

### Numbered Embodiments

Some embodiments provided herein are described by way of the following provided numbered embodiments:

1. A dual-specific bifunctional polypeptide that promotes proteasome-mediated degradation of at least one target protein, comprising:
a) a first targeting moiety that is capable of binding to at least one target protein,
b) a second targeting moiety that is capable of binding to at least one target protein,
c) a ubiquitin-proteasome system recruiting domain (URD),
d) optionally a first linker peptide, wherein the first linker peptide is positioned between the first targeting moiety and the URD, and
e) optionally a second linker peptide, wherein the second linker peptide is positioned between the second targeting moiety and the URD,
wherein proximity of the dual-specific bifunctional polypeptide to at least one target protein through binding of the first targeting moiety and/or the second targeting moiety induces ubiquitination of the at least one target protein via the URD, thereby promoting proteasome-mediated degradation of the at least one target protein.

2. The dual-specific bifunctional polypeptide of embodiment 1, wherein the first targeting moiety is designed to bind a first target protein and the second targeting moiety is designed to bind a second target protein.

3. The dual-specific bifunctional polypeptide of embodiment 1, wherein the first targeting moiety and the second targeting moiety are designed to bind to the same target protein.

4 The dual-specific bifunctional polypeptide of embodiment 3, wherein the first targeting moiety is capable of binding to a first binding site of the target protein and the second targeting moiety is capable of binding a second binding site of the target protein.

5. The dual-specific bifunctional polypeptide of embodiment 3, wherein the first targeting moiety and second targeting moiety are capable of binding the same binding site of the target protein, wherein the dual-specific bifunctional polypeptide is capable of engaging at least two molecules of the at least one target protein.

6 The dual-specific bifunctional polypeptide of any of embodiments 1-5, wherein the at least one target protein is selected from MYC, optionally c-MYC, beta catenin 1 (CTNNB1), and proliferating cell nuclear antigen (PCNA).

7. The dual-specific bifunctional polypeptide of any one of embodiments 1-6, wherein the first targeting moiety and/or the second targeting moiety bind, or bind at least in part, to at least a portion of:
a) the DNA binding domain of MYC; or
b) the armadillo domain of CTNNB1.

8. The dual-specific bifunctional polypeptide of any one of embodiments 1-7, wherein the first targeting moiety and/or second targeting moiety binds, or binds at least in part, to at least a portion of the target protein selected from the portion consisting of:
a) amino acids 54-406 of MYC (SEQ ID NO: 1),
b) amino acids 354-406 of MYC (SEQ ID NO: 1),
c) amino acids 408-437 of MYC (SEQ ID NO: 1),
d) amino acids 410-419 of MYC (SEQ ID NO: 1),
e) amino acids 150-663 of CTNNB1 (SEQ ID NO: 3),
f) amino acids 15-29 of CTNNB1 (SEQ ID NO: 3),
g) amino acids 249-265, 292-306, 379-390, 415-429, 462-470, or 505-519, or any combination thereof, of CTNNB1 (SEQ ID NO: 3), and
h) amino acids 38-48, 123-129, or 251-257 or any combination thereof, of PCNA (SEQ ID NO: 1836).

9. The dual-specific bifunctional polypeptide of any one of embodiments 1-8, wherein the first targeting moiety and/or the second targeting moiety is selected from an endogenous binding partner of the at least one target protein, antibody, Fab, F(ab′)₂, Fab′, scFv, sdAb, V_{H} domain, V_{L} domain, V_{H}H, V_{NAR}, diabody, intrabody, DARPin, monobody, affibody, avimer, or any fragment or derivative thereof.

10. The dual-specific bifunctional polypeptide of embodiment 9, wherein the first targeting moiety and/or the second targeting moiety is an sdAb, optionally comprising either a Vx domain or a V_{L} domain of an IgG antibody, or a V_{H}H, or V_{NAR} domain of a heavy chain antibody (HcAb)

11. The dual-specific bifunctional polypeptide of any one of embodiments 1-10, wherein the first targeting moiety and/or the second targeting moiety are less than or equal to 260, 259, 258, 257, 256, 255, 254, 253, 252, 251, 250, 245, 240, 235, 230, 225, 220, 215, 210, 205, 200, 195, 190, 185, 180, 175, 170, 165, 160, 155, 150, 145, 140, 135, 130, 129, 128, 127, 126, 125, 124, 123, 122, 121, 120, 119, 118, 117, 116, 115, 114, 113, 112, 110, 109, 108, 107, 106, 105, 104, 103, 102, 101, 100, 99, 98, 97, 96, 95, 94, 93, 92, 91, 90, 89, 88, 87, 86, 85, 84, 83, 82, 81, 80, 75, 70, 65, 60, 55, 54, 53, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 amino acids long, or a range defined by any two of the preceding values.

12. The dual-specific bifunctional polypeptide of any one of embodiments 1-11, wherein the first targeting moiety and/or the second targeting moiety are selected from 3DX, 3DX.1, 3DX.2, Omomyc, TCF4, TCF4.1, TCF4.2, TCF4.3, TCF4.4, TCF4.5, TCF4.6, TCF4.7, TCF4.8, TCF4.9, TCF4.10, TCF4.11, TCF4.12, TCF4.13, TCF4d1 peptide, TCF4K22R, TCF4K30R, TCF_reverse, TCF4_N, TCF4_C, BC2, con1, p21, p21.1, p21.2, p21.3, p21.4, p21.5, p21.6, p21.7, p21.8, and p21.9 or a fragment thereof that is capable of binding to the target protein.

13. The dual-specific bifunctional polypeptide of any one of embodiments 1-12, wherein the targeting moiety comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NO: 4-7, 9-11, 13, 2087-2090, 2094-2100, and 2104-2112.

14. The dual-specific bifunctional polypeptide of any one of embodiments 1-13, wherein the URD is derived from at least one E3 ubiquitin ligase.

15. The dual-specific bifunctional polypeptide of any one of embodiments 1-14, wherein the URD is derived from a RING family E3 ubiquitin ligase, an H-box family E3 ubiquitin ligase or a viral analogue of an E3 ubiquitin ligase.

16. The dual-specific bifunctional polypeptide of any one of embodiments 1-15, wherein the URD is derived from CHIP, RNF114, RNF125, RNF138, RNF166, NHLRC1, CBL-b (Y363E), TRIM21, E6AP, FBXW7 alpha, FBXW7 beta, beta-TRCP, Keap1, SPOP, DCAF1, or VIF, or a truncation or fragment thereof that retains ubiquitin-proteasome recruiting activity, optionally RNF125.2, SPOP.2 or SPOP.3.

17. The dual-specific bifunctional polypeptide of any one of embodiments 1-11, wherein the URD is selected from a RING domain, optionally RNF114, RNF125, RNF138, RNF166, NHLRC1, CBL-b (Y363E), or TRIM21, and an H-box (Cul4-DDB1) domain, optionally DCAF1, or VIF, or a truncation or fragment thereof that retains ubiquitin-proteasome recruiting activity, optionally RNF125.2.

18. The dual-specific bifunctional polypeptide of any one of embodiments 1-17, wherein the URD is selected from:
a) the URD derived from a protein which localizes to the cytoplasm, optionally selected from FBXW7 beta and beta-TRCP;
b) the URD derived from a protein which localizes to the nucleus, optionally selected from FBXW7 alpha and SPOP, optionally SPOP.2 or SPOP.3; and
c) the URD derived from a protein which localizes to the cytoplasm and the nucleus, optionally selected from CHIP, RNF114, RNF125, RNF138, RNF166, NHLRC1, CBL-b (Y363E), TRIM21, E6AP, Keap1, and DCAF1; or
d) or a truncation or fragment of the URD that retains ubiquitin-proteasome recruiting activity, optionally RNF125.2, SPOP.2 or SPOP.3;
optionally wherein the protein is an E3 ubiquitin ligase.

19. The dual-specific bifunctional polypeptide of any one of embodiments 1-18, wherein the URD is less than or equal to 370, 369, 368, 367, 366, 365, 364, 363, 362, 361, 360, 300, 250, 200, 175, 170, 165, 160, 155, 150, 145, 140, 135, 130, 129, 128, 127, 126, 125, 124, 123, 120, 115, 110, 105, 100, 95, 90, 85, 80, 75, 70, 65, 60, 55, 50, 47, 46, 45, 44, 43, 42, 41, 40, 39, 38, 37, 36, 35, 34, 33, 32, 31, 30, 25, or 20 amino acids long, or a range defined by any two of the preceding values.

20. The dual-specific bifunctional polypeptide of any one of embodiments 1-19, wherein the URD comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NO: 29-43, 1855-1856, and 1870, or a truncation or fragment thereof that retains ubiquitin-proteasome recruiting activity.

21. The dual-specific bifunctional polypeptide of any one of embodiments 1-20, wherein the first linker peptide and/or the second linker peptide are 2 to 20 amino acids in length.

22. The dual-specific bifunctional polypeptide of any one of embodiments 1-21, wherein the first linker peptide and/or the second linker peptide comprise or consist of glycine and serine.

23. The dual-specific bifunctional polypeptide of any one of embodiments 21-22, wherein the first linker peptide and/or second linker peptide comprises or consists of the amino acid sequence of any one of SEQ ID NO: 44-49.

24. The dual-specific bifunctional polypeptide of any one of embodiments 1-23, further comprising at least one nuclear localization signal (NLS) peptide.

25. The dual-specific bifunctional polypeptide of embodiment 24, wherein at least one NLS peptide is derived from MYC NLS, SV40 NLS, 3XMYC NLS, 3xSV40 NLS, SPOP NLS, nucleoplasmin NLS, 53BP1 NLS, or Hrp1 NLS.

26. The dual-specific bifunctional polypeptide of embodiment 25, wherein at least one NLS comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NO: 51, and 1897-1902.

27. The dual-specific bifunctional polypeptide of any one of embodiments 1-26, wherein the at least one NLS peptide is located at the N-terminus of the dual-specific bifunctional polypeptide, the C-terminus of the dual-specific bifunctional polypeptide, is part of the URD, optionally an endogenous NLS of the URD, or is part of the first and/or second targeting moiety, optionally an endogenous NLS of the first and/or second targeting moiety.

28. The dual-specific bifunctional polypeptide of embodiment 1-27, wherein a first NLS peptide is located at the N-terminus of the dual-specific bifunctional polypeptide and a second NLS peptide is located at the C-terminus of the dual-specific bifunctional polypeptide.

29. The dual-specific bifunctional polypeptide of any one of embodiments 1-28, wherein the orientation of the first targeting moiety relative to the URD is the same orientation as compared to the naturally occurring substrate recognition domain of the protein from which the URD is derived.

30. The dual-specific bifunctional polypeptide of any one of embodiments 1-29, wherein the orientation of the first targeting moiety relative to the URD is the opposite orientation as compared to the naturally occurring substrate recognition domain of the protein from which the URD is derived.

31. The dual-specific bifunctional polypeptide of any one of embodiments 1-30, wherein the orientation of the second targeting moiety relative to the URD is the same orientation as compared to the naturally occurring substrate recognition domain of the protein from which the URD is derived.

32. The dual-specific bifunctional polypeptide of any one of embodiments 1-31, wherein the orientation of the second targeting moiety relative to the URD is the opposite orientation as compared to the naturally occurring substrate recognition domain of the protein from which the URD is derived.

33. The dual-specific bifunctional polypeptide of any one of embodiments 1-32, wherein the first targeting moiety is N-terminal relative to the URD.

34. The dual-specific bifunctional polypeptide of any one of embodiments 1-33, wherein the second targeting moiety is C-terminal relative to the URD.

35. The dual-specific bifunctional polypeptide of any one of embodiments 1-34, further comprising an epitope tag, optionally a hemagglutinin (HA) tag, optionally wherein the HA tag comprises or consists of the amino acid sequence of SEQ ID NO: 50, wherein the epitope tag can be used to visualize the dual-specific bifunctional polypeptide.

36. The dual-specific bifunctional polypeptide of any one of embodiments 1-35, wherein the polypeptide is less than or equal to 60, 70, 80, 90, 100, 110, 120, 130, 140, 150, 160, 170, 180, 190, 200, 250, 300, 350, 400, 450, 500, 550, 600, 650, 700, 750, 800, 850, 900, 950. 1000, 1050, 1100, or 1150, amino acids long, or a range defined by any two of the preceding values.

37. The dual-specific bifunctional polypeptide of any one of embodiments 1-29, wherein the dual-specific bifunctional polypeptide:
a) comprises an amino acid sequence having at least 80%, 85%, 86%, 87%, 88%, 89%, 90%, 91%, 92%, 93%, 94%, 95%, 96%, 97%, 98%, 99%, or 100% sequence identity to:
   i) the peptide of any one of SEQ ID NO: 72-1165, or the TBD, URD, NLS, and/or linker portion thereof, optionally without the HA tag sequence, or
   ii) the peptide, or the TBD, URD, NLS, and/or linker portion thereof, optionally without the HA tag sequence, encoded by the polynucleotide of any one of SEQ ID NO: 1188-1835; or
b) comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to the peptide of any one of SEQ ID NO:2287-2351.

38. The dual-specific bifunctional polypeptide of any one of embodiments 1-36, wherein the at least one target protein comprises MYC.

39. The dual-specific bifunctional polypeptide of embodiment 38, wherein the URD is the URD of CHIP, DCAF1, E6AP, FBXW7a, FBXW7b, beta-TRCP, Keap1, RNF114, RNF125, RNF138, NHLRC1, CBL-b (Y363E), TRIM21, SPOP or VIF, or a truncation or fragment thereof that retains ubiquitin-proteasome recruiting activity, optionally RNF125.2, SPOP.2 or SPOP.3.

40. The dual-specific bifunctional polypeptide of embodiment 38 or 39, wherein the first targeting moiety and/or second targeting moiety is 3DX.1, or optionally an amino acid sequence having at least 80%, 85%, 86%, 87%, 88%, 89%, 90%, 91%, 92%, 93%, 94%, 95%, 96%, 97%, 98%, 99%, or 100% sequence identity to SEQ ID NO: 4.

41. The dual-specific bifunctional polypeptide of embodiment 40, wherein the dual-specific bifunctional polypeptide comprises an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NO: 72-155, or the TBD, URD, NLS, and/or linker portion thereof, optionally without the HA tag sequence.

42. The dual-specific bifunctional polypeptide of embodiment 38 or 39, wherein the first targeting moiety and/or second targeting moiety is 3DX.2, or optionally an amino acid sequence having at least 80%, 85%, 86%, 87%, 88%, 89%, 90%, 91%, 92%, 93%, 94%, 95%, 96%, 97%, 98%, 99%, or 100% sequence identity to SEQ ID NO: 5.

43. The dual-specific bifunctional polypeptide of embodiment 42, wherein the dual-specific bifunctional polypeptide comprises an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NO: 156-239, or the TBD, URD, NLS, and/or linker portion thereof, optionally without the HA tag sequence.

44. The dual-specific bifunctional polypeptide of embodiment 38 or 39, wherein the first targeting moiety and/or the second targeting moiety is Omomyc, or optionally an amino acid sequence having at least 80%, 85%, 86%, 87%, 88%, 89%, 90%, 91%, 92%, 93%, 94%, 95%, 96%, 97%, 98%, 99%, or 100% sequence identity to SEQ ID NO: 6.

45. The dual-specific bifunctional polypeptide of embodiment 44, wherein the dual-specific bifunctional polypeptide:
a) comprises an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NO:240-323, or the TBD, URD, NLS, and/or linker portion thereof, optionally without the HA tag sequence; or
b) comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NO:2287-2288, 2295, 2301, 2318, 2321, and 2333-2335.

46. The dual-specific bifunctional polypeptide of any one of embodiments 1-36, wherein the at least one target protein comprises CTNNB1.

47. The dual-specific bifunctional polypeptide of embodiment 46, wherein the URD is the URD of CHIP, DCAF1, E6AP, FBXW7a, FBXW7b, beta-TRCP, Keap1, RNF114, RNF125, RNF138, NHLRC1, CBL-b (Y363E), TRIM21, SPOP, or VIF, or a truncation or fragment thereof that retains ubiquitin-proteasome recruiting activity, optionally RNF125.2, SPOP.2 or SPOP.3.

48. The dual-specific bifunctional polypeptide of embodiment 46 or 47, wherein the first targeting moiety and/or second targeting moiety is TCF4.1, TCF4.2, TCF4.3, TCF4.4, TCF4.5, TCF4.6, TCF4.7, TCF4.8, TCF4.9, TCF4.10, TCF4.11, TCF4.12, TCF4.13, TCF4d1 peptide, TCF4K22R, TCF4K30R, TCF_reverse, TCF4_N, or TCF4_C, or optionally an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NOs: 11, 1841-1847, 1931-1936, or 1939-1943.

49. The dual-specific bifunctional polypeptide of embodiment 48, wherein the dual-specific bifunctional polypeptide:
a) comprises an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NO: 720-1009, or the TBD, URD, NLS, and/or linker portion thereof, optionally without the HA tag sequence, or the TBD, URD, NLS, and/or linker portion thereof, optionally without the HA tag sequence;
b) comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NO: 1849-1894, 1898-1911, or 1944-1945.

50. The dual-specific bifunctional polypeptide of embodiment 46 or 47, wherein the first targeting moiety and/or second targeting moiety is BC2, or optionally an amino acid sequence having at least 80%, 85%, 86%, 87%, 88%, 89%, 90%, 91%, 92%, 93%, 94%, 95%, 96%, 97%, 98%, 99%, or 100% sequence identity to SEQ ID NO: 13.

51. The dual-specific bifunctional polypeptide of embodiment 50, wherein the dual-specific bifunctional polypeptide comprises an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NO: 1010-1165, or the TBD, URD, NLS, and/or linker portion thereof, optionally without the HA tag sequence.

52. The dual-specific bifunctional polypeptide of any one of embodiments 1-36, wherein the at least one target protein comprises PCNA.

53. The dual-specific bifunctional polypeptide of embodiment 52, wherein the URD is the URD of CHIP, DCAF1, E6AP, FBXW7a, FBXW7b, beta-TRCP, Keap1, RNF114, RNF125, RNF138, NHLRC1, CBL-b (Y363E), TRIM21, SPOP, or VIF, or a truncation or fragment thereof that retains ubiquitin-proteasome recruiting activity, optionally RNF125.2, SPOP.2 or SPOP.3.

54. The dual-specific bifunctional polypeptide of embodiment 52 or 53, wherein the first targeting moiety and/or second targeting moiety is con1, or optionally an amino acid sequence having at least 80%, 85%, 86%, 87%, 88%, 89%, 90%, 91%, 92%, 93%, 94%, 95%, 96%, 97%, 98%, 99%, or 100% sequence identity to SEQ ID NO: 1921.

55. The dual-specific bifunctional polypeptide of embodiment 54, the dual-specific bifunctional polypeptide comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NO: 1851-1856, 1864-1879, 1884-1897, 1907-1911, or 1944-1945.

56. The dual-specific bifunctional polypeptide of embodiment 52 or 53, wherein the first targeting moiety and/or second targeting moiety is p21.1, p21.2, p21.3, p21.4, p21.5, p21.6, p21.7, p21.8, or p21.9, or optionally an amino acid sequence having at least 80%, 85%, 86%, 87%, 88%, 89%, 90%, 91%, 92%, 93%, 94%, 95%, 96%, 97%, 98%, 99%, or 100% sequence identity to SEQ ID NO: 1912-1920.

57. The dual-specific bifunctional polypeptide of embodiment 56, the dual-specific bifunctional polypeptide comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NO: 1898-1906.

58. The dual-specific bifunctional polypeptide of any one of embodiments 1-36, 38-45 and 46-51 wherein the first target protein is c-MYC and the second target protein is CTNNB1, or the first target protein is CTNNB1 and the second target protein is c-MYC.

59. The dual-specific bifunctional polypeptide of any one of embodiments 1-36, 38-45 and 52-55, wherein the first target protein is c-MYC and the second target protein is PCNA, or wherein the first target protein is PCNA and the second target protein is c-MYC.

60. The dual-specific bifunctional polypeptide of any one of embodiments 1-36, 46-51 and 52-55, wherein the first target protein is CTNNB1 and the second target protein is PCNA, or wherein the first target protein is PCNA and the second target protein is CTNNB1.

61. The dual-specific bifunctional polypeptide of any one of embodiments 58-60, wherein the dual-specific bifunctional polypeptide comprises or consists of an amino acid sequence having at least 80%, 85%, 86%, 87%, 88%, 89%, 90%, 91%, 92%, 93%, 94%, 95%, 96%, 97%, 98%, 99%, or 100% sequence identity to any one of SEQ ID NO: 1849-1911 or 1944-1945.

62. A polynucleotide encoding for a polypeptide having at least 80%, 85%, 86%, 87%, 88%, 89%, 90%, 91%, 92%, 93%, 94%, 95%, 96%, 97%, 98%, 99%, or 100% sequence identity to the dual-specific bifunctional polypeptide of any one of embodiments 1-61.

63. The polynucleotide of embodiment 62, wherein the polynucleotide is a DNA or an RNA, optionally an mRNA or a circular RNA.

64. The polynucleotide of embodiment 62 or 63, wherein the polynucleotide is packaged in a lipid nanoparticle or a viral vector, optionally an adenovirus, adeno-associated virus, lentivirus, or retrovirus vector.

65. A pharmaceutical composition comprising the dual-specific bifunctional polypeptide or polynucleotide of any one of embodiments 1-64 and one or more pharmaceutically acceptable excipients, carriers, or diluents.

66. The dual-specific bifunctional polypeptide, polynucleotide, or pharmaceutical composition of any one of embodiments 1-65 for use in the treatment of a cancer in a patient in need thereof.

67. The dual specific bifunctional polypeptide, polynucleotide, or pharmaceutical composition of any one of embodiments 1-66 for use in the manufacture of a medicament, optionally for the treatment of cancer.

68. A method of treating a subject comprising administering the dual-specific bifunctional polypeptide, polynucleotide, or pharmaceutical composition of any one of embodiments 1-67 to a subject in need thereof, optionally wherein the subject has a cancer.

69. A method of reducing the amount of a target protein in a cell comprising contacting the cell with the dual-specific bifunctional polypeptide, polynucleotide, or pharmaceutical composition of any one of embodiments 1-68, optionally wherein the first and/or second target protein is selected from MYC, optionally c-MYC, beta catenin 1 (CTNNB1), and proliferating cell nuclear antigen (PCNA).

70. The method of embodiment 69, wherein said cell is in a subject, and said contacting comprises administering the dual-specific bifunctional polypeptide, polynucleotide, or pharmaceutical composition of any one of embodiments 1-67 to the subject, optionally wherein the subject has cancer.

71. The method of embodiment 69, wherein the cell is ex vivo, and said contacting comprises contacting the dual-specific bifunctional polypeptide, polynucleotide, or pharmaceutical composition of any one of embodiments 1-67 to the cell ex vivo, optionally in an adoptive cell therapy.

72. The method of embodiment 71, further comprising administering the cell to a subject after the contacting step, optionally wherein the subject is also the source of the cell.

73. The dual-specific bifunctional polypeptide, polynucleotide, pharmaceutical composition, or method of any one of the preceding embodiments, wherein the dual-specific bifunctional polypeptide promotes proteasome-mediated degradation of the first and/or second target protein when measured in one or more assays described in Example 1, for example, by Western blot, optionally wherein the proteasome-mediated degradation is inhibited when the cells comprising the target protein and the dual-specific bifunctional polypeptide are treated with a proteasome inhibitor.

74. The dual-specific bifunctional polypeptide, polynucleotide, pharmaceutical composition, or method of embodiment 73, wherein treatment of the cells comprising the first and/or second target protein and the dual-specific bifunctional polypeptide with a proteasome inhibitor reduces the amount of degradation of the target protein by at least 50%, 60%, 70%, 80%, 90%, 95% or 100% as compared to cells not treated with the proteasome inhibitor.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

### Example 1: HiBiT™ Assay for Proteasomal-Mediated Degradation of Target Protein(s)

To obtain a preliminary assessment of the effectiveness of dual-specific bifunctional polypeptide degraders, HepG2 cells were transfected with plasmids for transiently expressing HiBiT™-tagged target proteins and for expressing the corresponding dual-specific bifunctional polypeptide degrader at the time of plating. Doxycycline was added 24 hours post-transfection to induce expression of the dual-specific bifunctional polypeptide degrader from the doxycycline-inducible system. HiBiT™ signal was measured the following day using Nano-Glo HiBiT™ lytic detection system according to manufacturer's protocol. Table 16 summarizes the assay conditions.

TABLE 16
Assay Conditions

| Assay # | Targets | Cell line | Target Expression | SEQ ID NO |
|---|---|---|---|---|
| 1 | CTNNB1/CTNNB1 | HepG2 | Exogenous plasmid | All those listed in |
| | CTNNB1/PCNA | | | FIG. 9A-9C |
| | CTNNB1/MYC | | | |
| | MYC/MYC | | | |

The results of the HiBiT™ assays are summarized in Tables 7, 9, and 11 above, and FIG. 9A-9C. Average values of the percentage of target protein remaining relative to control was determined. A grade for the percent of degradation is provided based on the following cutoffs: A>50% degradation (<50% remaining); B≥20%-≤50% degradation (≥50%-≤80% remaining); C≥2%-<20% degradation (>80%-≤98 remaining); and D<2% degradation (>98% remaining) relative to control. “n.a.” means not applicable where both targeting moieties target the same target protein, and “not tested” or “ ” indicates that the degradation of the target protein was not tested. For dual-specific degraders where both targeting moieties are directed to the same target protein, the “Combined Grade” is a single letter.

### Example 2: Western Blot Assay for Proteasomal-Mediated Degradation of Target Protein

To obtain a preliminary assessment of the effectiveness of dual-specific bifunctional polypeptide degraders, a western blot assay was performed for select dual-specific bifunctional polypeptide degraders. Doxycycline-inducible expression of dual-specific bifunctional polypeptide degrader was introduced by transient expression via plasmid or stable expression by viral transduction in HepG2. The following table summarizes the conditions for the several assay conditions utilized.

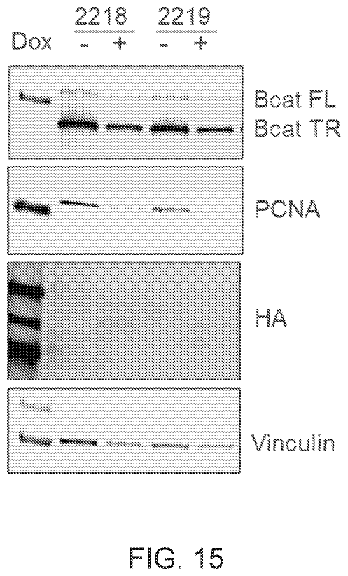

TABLE 17
Assay Conditions

| Assay # | Targets | Cell line | Target Expression | SEQ ID NO |
|---|---|---|---|---|
| 2 | CTTNB1/PCNA | HepG2 | Exogenous plasmid | FIGS. 15, 17 |
| 3 | CTTNB1/PCNA | HepG2 | Stable cell line | FIGS. 12, 14, 19 |
| | CTTNB1/MYC | | | |

For assay #2, Cells were seeded in 6 wells plate and transfected at the time of plating with plasmid that expresses doxycycline-inducible dual-specific bifunctional polypeptide Doxycycline was added 24 hours post-transfection, and cells were harvested the following day by scraping or trypsinization. Pelleted cells were lysed in RIPA buffer supplemented with protease and proteasome inhibitors for 30 minutes on ice and lysates were cleared by centrifugation. Protein levels were measured by BCA assay and equal amounts of lysates were loaded to 4-15% pre-cast gel. Gels were transferred to nitrocellulose membrane using a semi-dry method and incubated overnight in primary antibodies. Following secondary antibody incubation, membranes were developed using the LiCor imaging system.

For assay #3, stable cell line was established by transduction of viral supernatant containing dual-specific bifunctional polypeptide in HepG2 cells. Upon selection in puromycin for 72 hours, stable lines were established. For western blot, stable cell lines were seeded in 6 wells plate and doxycycline was added 24 hours post-transfection, and cells were harvested the following day by scraping or trypsinization. Pelleted cells were lysed in RIPA buffer supplemented with protease and proteasome inhibitors for 30 minutes on ice and lysates were cleared by centrifugation. Protein levels were measured by BCA assay and equal amounts of lysates were loaded to 4-15% pre-cast gel. Gels were transferred to nitrocellulose membrane using a semi-dry method and incubated overnight in primary antibodies. Following secondary antibody incubation, membranes were developed using the LiCor imaging system.

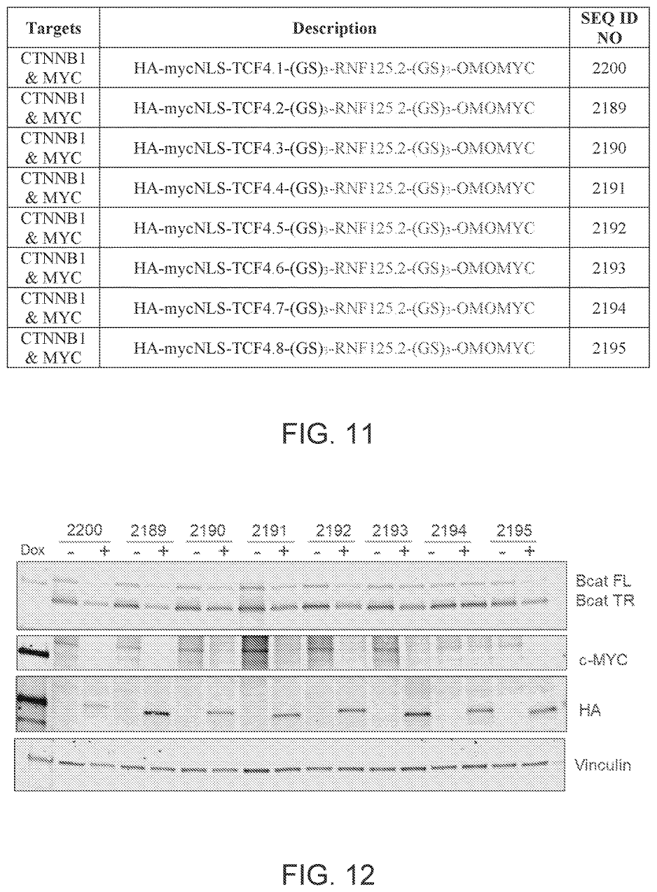

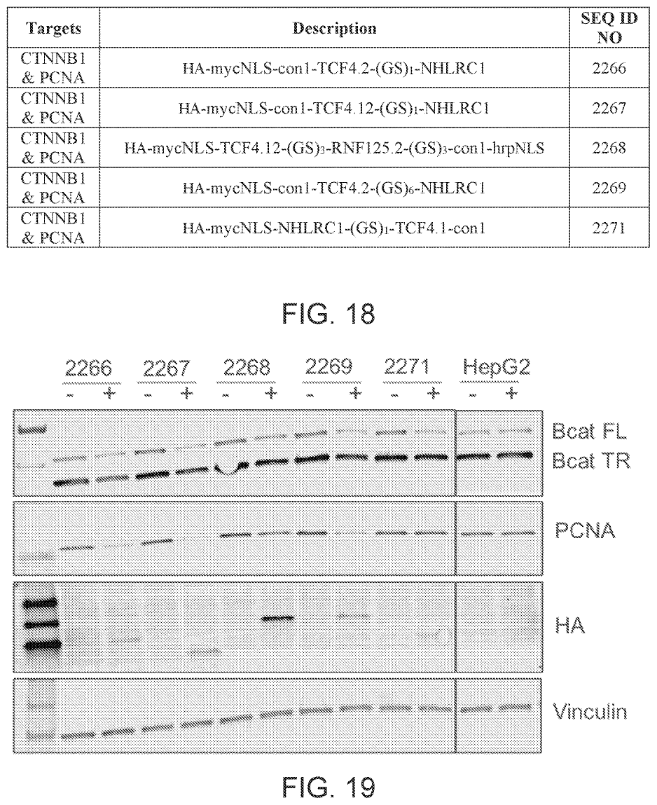

FIG. 11-19 are examples of western blot experiments. Western blot analysis demonstrated degradation of the target proteins by the dual-specific bifunctional polypeptide degraders at varying levels. Note that FIGS. 18 and 19 demonstrate that “tandem” dual-specific bifunctional polypeptide degraders having a CTNNB1 targeting moiety and a PCNA targeting moiety both located on the N-terminal side of the URD were effective at degrading CTNNB1 and PCNA. In FIG. 11-19, “Dox” “+” or “−” signifies the presence or absence respectively of doxoxycycline to induce expression of the dual-specific bifunctional polypeptide degrader. Expression of the dual-specific bifunctional polypeptide degrader was confirmed by labeling the HA tag of the degrader with an HA antibody (HA lane), and vinculin was used as a control to normalize protein loading (Vinculin lane). The target protein lanes are labeled accordingly, with “Bcat FL” signifying CTNNB1 full-length, and “Bcat TR” signifying CTNNB1 truncated form.

### Example 3: Subcellular Localization of Mono-Specific Bifunctional Polypeptide Degraders

Cells were seeded and transfected as described in Example 1. 48 hours post-plating, cells were fixed in 4% paraformaldehyde solution for 10 minutes, rinsed once in PBS pH 7.4, and permeabilized in 0.1% triton x-100 for 15 minutes at room temperature. To stain for mono-specific bifunctional polypeptide degraders, cells were incubated overnight with HA antibody in PBS containing 1% BSA and 0.1% triton x-100 to label the HA tag of the mono-specific bifunctional polypeptide degraders. The next day, cells were rinsed thrice with PBS and stained with fluorophore-tagged secondary antibody. Nucleus was counterstained with Hoescht33342. Cells were imaged on an inverted microscope with an LED fluorescence source.

Several NLS sequences were tested in various configurations. The following table provides a description of the NLS sequences tested.

TABLE 18
Exemplary NLS sequences used in Example 3

| | | Uniprot ID of | |
|---|---|---|---|
| Name | Parental Protein | Parental Protein | SEQ ID NO |
| spNLS.2 | SPOP | O43791 | 1896 |
| spNLS | SPOP | O43791 | 1897 |
| hrpNLS | Hrp1 | Q99383 | 1898 |
| bpNLS | 53BP1 | Q12888 | 1899 |
| nlpNLS | Nucleoplasmin | P05221 | 1900 |
| 3x(svNLS) | SV40 | P03070 | 1901 |
| 3x(mycNLS) | MYC | P01106 | 1902 |
| svNLS | SV40 | P03070 | 1903 |
| mycNLS | MYC | P01106 | 1904 |

The results are summarized in the following Table, where “Nuc” is predominantly nucleus, “Cyt” is predominantly cytoplasm, and “Mix” is both nucleus and cytoplasm. HA is the hemagglutinin epitope tag. The URDs and linkers are described elsewhere herein.

TABLE 19
Subcellular localization of exemplary polypeptides

| | SEQ | | |
|---|---|---|---|
| URD | ID | | Loca- |
| Class | NO | Description | tion |
| SPOP | 2170 | HA-con1.1-SPOP.2 | Nuc |
| | 2381 | HA-con1.1-SPOP.3 | Cyt |
| | 2382 | HA-con1.1-SPOP.3-mycNLS | Nuc |
| | 2383 | HA-con1.1-SPOP.3-3xmycNLS | Nuc |
| | 2384 | HA-con1.1-SPOP.3-svNLS | Nuc |
| | 2385 | HA-con1.1-SPOP.3-3xsvNLS | Nuc |
| | 2386 | HA-con1.1-SPOP.3-nlpNLS | Nuc |
| | 2387 | HA-con1.1-SPOP.3-bpNLS | Nuc |
| | 2388 | HA-con1.1-SPOP.3-hrpNLS | Nuc |
| NHLRC1 | 2353 | HA-mycNLS-TCF4.2-(GS)₆-NHLRC1.1 | Nuc |
| | 2352 | HA-TCF4.2-(GS)₆-NHLRC1.1 | Mix |
| | 2354 | HA-TCF4.2-(GS)₆-NHLRC1.1-spNLS | Nuc |
| | 2372 | HA-nlpNLS-mycV.1500-d-NHLRC1.1 | Nuc |
| | 2367 | HA-mycNLS-mycV.1500-GGGS-NHLRC1.1 | Mix |
| RNF125 | 2362 | HA-3xsvNLS-RNF125.2-(GGGS)₂-mycV.1500 | Mix |
| | 2363 | HA-nlpNLS-RNF125.2-(GGGS)₁-mycV.1500 | Nuc |
| | 2364 | HA-mycNLS-RNF125.2-GGGS-mycV.1500 | Nuc |
| | 2365 | HA-nlpNLS-RNF125.2-GGGS-mycV.1500 | Mix |
| | 2366 | HA-hrpNLS-RNF125.2-GGGS-mycV.1500 | Mix |
| | 2374 | HA-spNLS.2-RNF125.2-(GGGS)₁-mycV.1500 | Mix |
| | 2375 | HA-RNF125.2-(GGGS)₁-mycV.1500-mycNLS | Mix |
| | 2376 | HA-RNF125.2-(GGGS)₁-mycV.1500-nlpNLS | Mix |
| | 2377 | HA-RNF125.2-(GGGS)₁-mycV.1500-hrpNLS | Nuc |
| | 2378 | HA-RNF125.2-(GGGS)₁-mycV.1500-spNLS.2 | Mix |
| RNF4 | 2368 | HA-nlpNLS-mycV.1500-GGGS-RNF4.1 | Nuc |
| | 2370 | HA-nlpNLS-mycV.1500-d-RNF4.1 | Nuc |
| | 2373 | HA-spNLS.2-mycV.1500-GGGS1-RNF4.1 | Nuc |
| RNF12.2 | 2371 | HA-nlpNLS-mycV.1500-d-RNF12.2 | Nuc |
| | 2369 | HA-nlpNLS-mycV.1500-GGGS-RNF12.2 | Nuc |
| | 2379 | HA-mycNLS-mycV.1500-GGGS-ZNRF1.1 | Nuc |
| | 2380 | HA-nlpNLS-mycV.1500-GGGS-ZNRF1.1 | Nuc |
| CHIP | 2361 | VL12.3-(GS)₃-CHIP.1-mycNLS-HA | Nuc |
| FBXW7a | 2356 | HA-FBXW7a.1-(GS)₃-VL12.3 | Nuc |
| SPOP | 2359 | VL12.3-(GS)₃-SPOP.1-mycNLS-HA | Nuc |
| VIF | 2360 | VL12.3-(GS)₃-VIF.1-mycNLS-HA | Mix |
| FBXW7b | 2357 | HA-FBXW7b.1-(GS)₃-VL12.3 | Cyt |
| ctrl | 2358 | HA-mycNLS-VL12.3 | Mix |
| ctrl | 2355 | HA-VL12.3 | Mix |

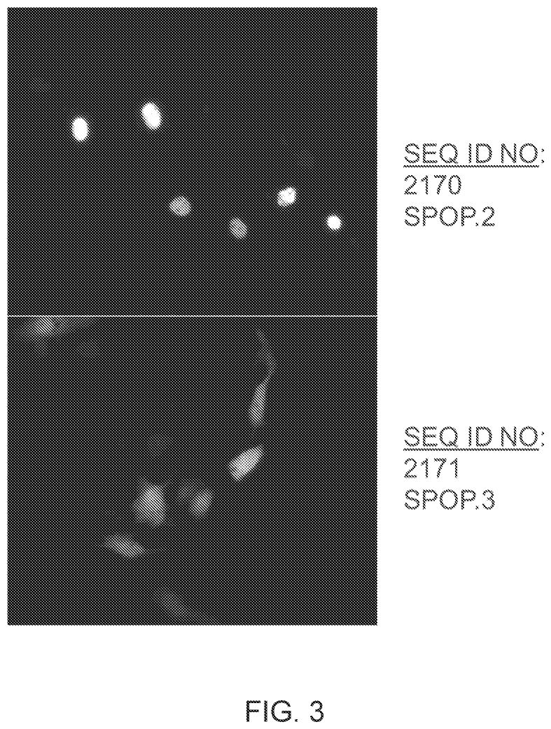

The SPOP URD is derived from an E3 ubiquitin ligase. SPOP.2 and SPOP.3 variant URDs are truncated regions of the full-length SPOP URD. Compared to SPOP.2, SPOP.3 has a truncation of 14 residues at the C-terminus. The additional 14 C-terminal residues of SPOP.2 function as an endogenous nuclear localization signal (NLS). As summarized in Table 19 above, subcellular localization was observed for mono-specific bifunctional polypeptides with a con1 targeting moiety and either a SPOP.2 URD (HA-con1.1-SPOP.2, SEQ ID NO: 3236) or SPOP.3 URD (HA-con1.1-SPOP.3, SEQ ID NO: 3237). Truncation of the 14 residues changes the subcellular localization of SPOP from the nucleus (SPOP.2) to the cytoplasm (SPOP.3). Microscopy data confirms that the SPOP.2 mono-specific bifunctional polypeptide is localized in the nucleus whereas the SPOP.3 mono-specific bifunctional polypeptide is localized in the cytoplasm (FIG. 3). This demonstrates the ability to direct polypeptides to subcellular compartments by modifying/deleting endogenous localization sequences.

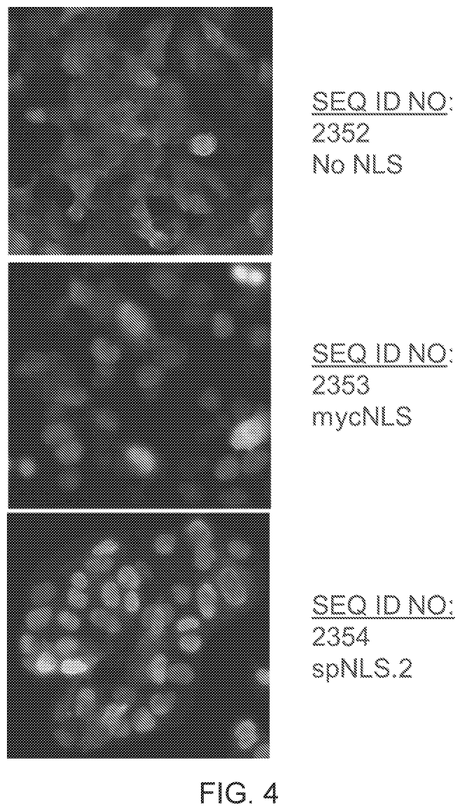

NHLRC1 is a RING E3 ubiquitin ligase with a bioinformatically-predicted nuclear localization and export signal (NLS and NES, Table 4). Mono-specific bifunctional polypeptides with a TCF4 variant targeting moiety showed expression throughout the cells that is not exclusive to the nucleus (SEQ ID NO: 2417). Incorporation of mycNLS or spNLS.2 in the sequence of the mono-specific bifunctional polypeptides (SEQ ID NO: 2401 and 2426, respectively) resulted in nuclear localization (FIG. 4). This demonstrates the ability to alter localization of polypeptides to subcellular compartments of interest by incorporating a localization sequence specific to such subcellular compartment of interest.

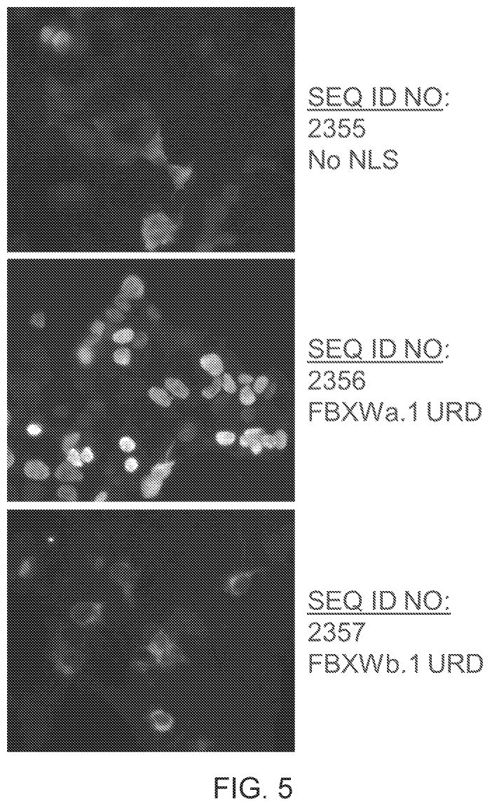

VL12.3 targeting moiety (SEQ ID NO: 57) showed localization throughout the cells when expressed as a standalone intrabody (FIG. 5). Mono-specific bifunctional polypeptides comprising VL12.3 targeting moiety and URDs derived from either FBXW7a or FBW7b, E3 ligases with known nuclear and cytoplasmic localization (Yeh et al. “FBXW7: a critical tumor suppressor of human cancers,” Molecular Cancer (2018) 17:115), respectively directed the mono-specific bifunctional polypeptide exclusively the nucleus (SEQ ID NO: 2544) or to the cytoplasm (SEQ ID NO: 2592). This highlights the ability to design mono-specific bifunctional polypeptides to desired subcellular localization through URD selection.

### Example 4: Subcellular Localization of Dual-Specific Bifunctional Polypeptide Degraders

Cell assays were prepared as described in Example 3.

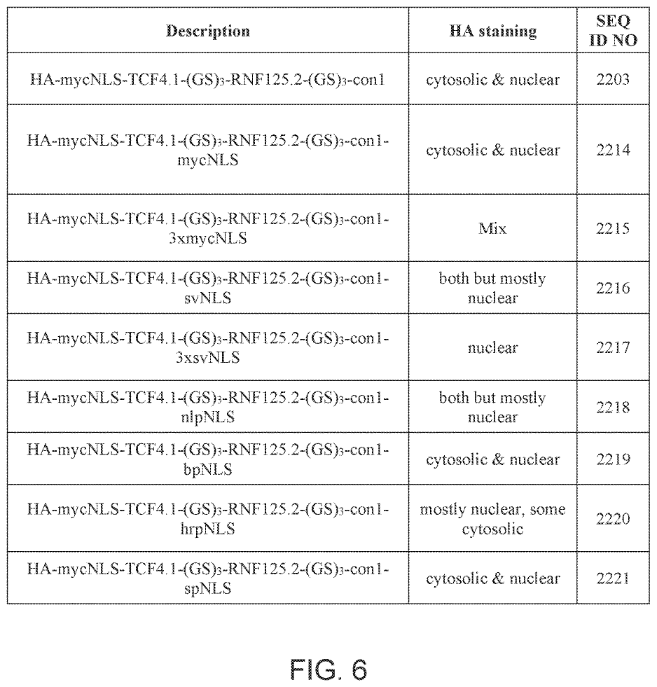

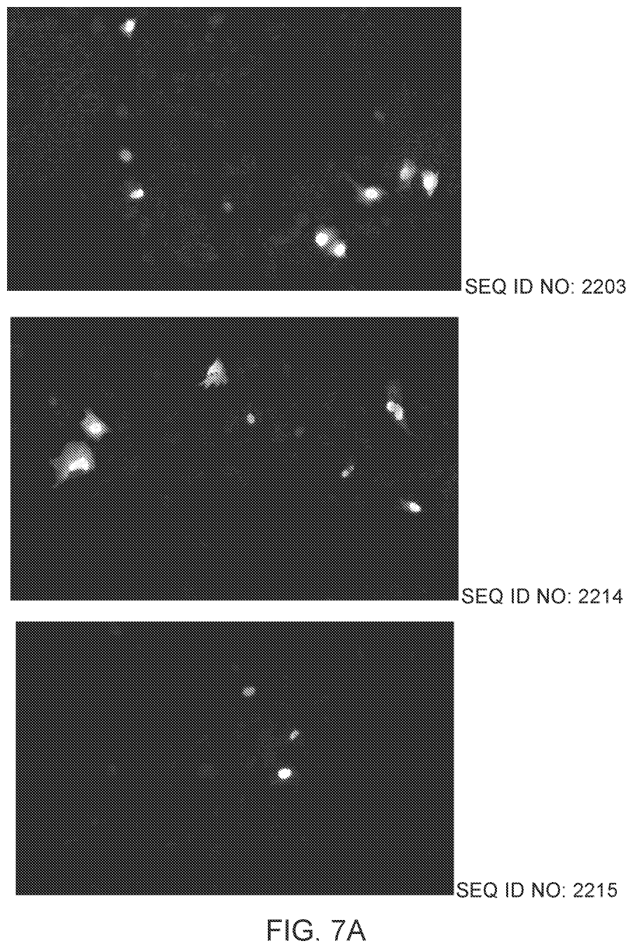

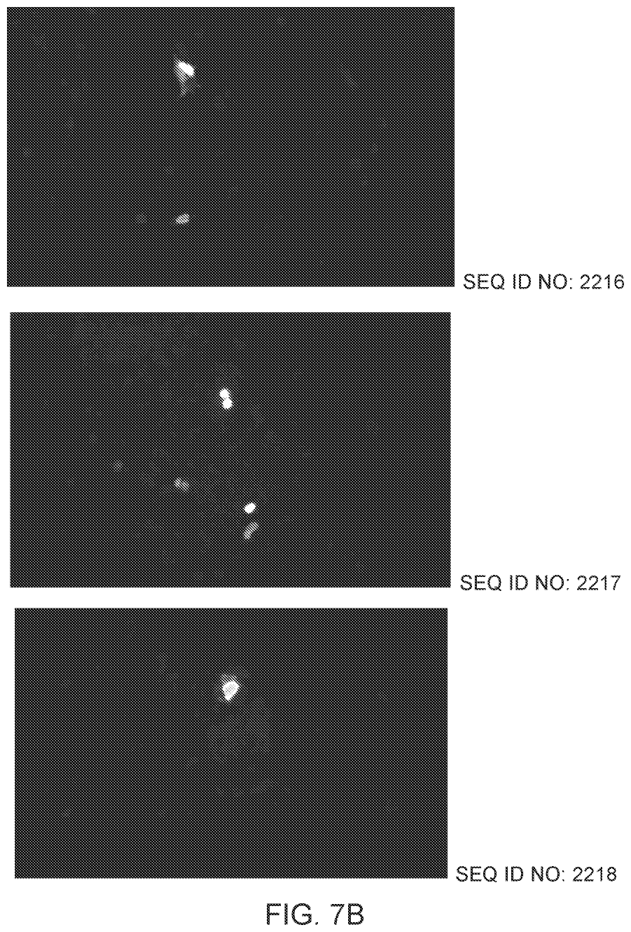

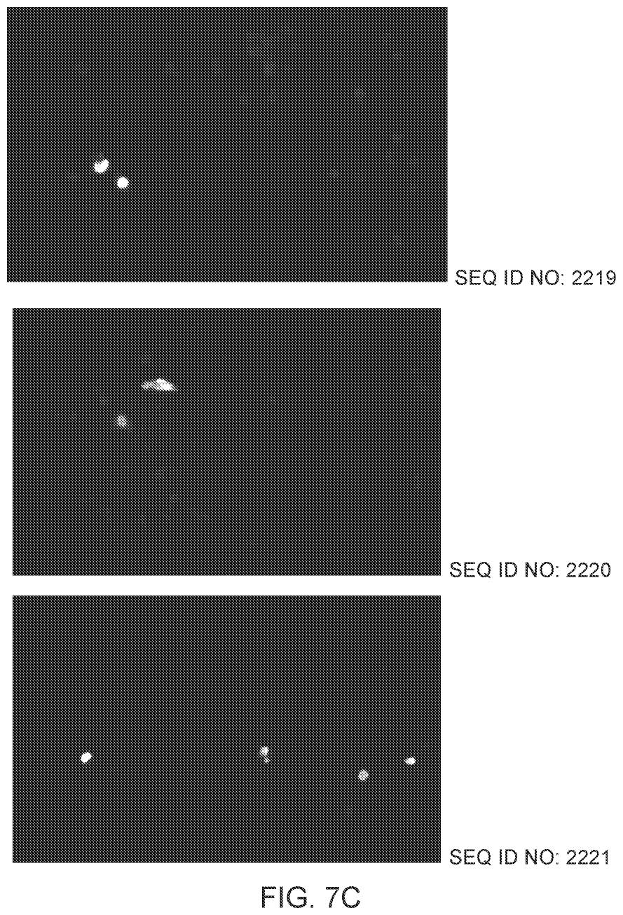

Several exemplary dual-specific bifunctional polypeptides were expressed and analyzed for the amount of peptide expression as well as whether the expression was localized to the cytosol, nucleus, or both. The dual-specific bifunctional polypeptide of SEQ ID NO: 2203 has an N-terminal mycNLS sequence, but no C-terminal NLS sequence. Various NLS sequences were added to the C-terminus. The results show that adding additional NLS sequences to the C-terminus of can improve nuclear localization, The results of the experiment are summarized in FIG. 6, and exemplary photomicrographs are shown in FIG. 7A-7C.

### Example 5. Dual-Specific Bifunctional Polypeptides that Direct Proteasomal-Mediated Degradation for Treatment of Cancer

A patient is selected who has a cancer. The cancer may be a brain cancer, breast cancer, colorectal cancer, kidney cancer, liver cancer, lung cancer, pancreatic cancer, bladder cancer, stomach cancer, or a hematological malignancy, or another cancer not listed here. The cancer may be non-responsive to standard of care treatments. The cancer may be primary and/or metastatic.

Any of the dual-specific bifunctional polypeptides, polynucleotides that encode for the dual-specific bifunctional polypeptides, or a pharmaceutical composition that comprises the dual-specific bifunctional polypeptide or polynucleotide and is specific for a protein target associated with cancer (e.g., MYC, CTNNB1, or PCNA) is administered to the patient. The dual-specific bifunctional polypeptide, polynucleotide, or pharmaceutical composition is administered enterally, parenterally, intravenously, intratumorally, intramuscularly, intra-arterially, intradermally, subcutaneously, intraperitoneally, intraventricularly, intrathecally, or intracranially. In some embodiments, the dual-specific bifunctional polypeptide, polynucleotide, or pharmaceutical composition is administered as at least one dose at an amount of 1, 10, 100, 1000 ng, or 1, 10, 50, 100, 200, 300, 400, 500, 600, 700, 800, 900, 1000 μg, or 1, 10, 100, 200, 300, 400, 500, 600, 700, 800, 900, 1000 mg, or any amount within a range defined by any two of the aforementioned amounts, or any other amount appropriate for optimal efficacy in humans. In some embodiments, the dual-specific bifunctional polypeptide, polynucleotide, or pharmaceutical composition is administered in a number of doses sufficient to achieve an improvement in the disease pathology in the patient. In some embodiments, the dual-specific bifunctional polypeptide, polynucleotide, or pharmaceutical composition is administered in 1, 2, 3, 4, 5, 6, 7, 8, 9, 10, 20, 30, 40, 50, 60, 70, 80, 90, 100, 110, 120, 130, 140, or 150 doses, or any number of doses within a range defined by any two of the aforementioned number of doses, for example, 1-150 doses, 1-10 doses, 1-50 doses, 50-100 doses, or 50-150 doses. In some embodiments, the dual-specific bifunctional polypeptide, polynucleotide, or pharmaceutical composition is administered in a single dose (for example, when using a gene therapy or neoadjuvant therapy approach). In the case of multiple doses, the doses can be administered every 1, 2, 3, 4, 5, 6, 7, 8, 9, 10, 11, 12, 24, 36, or 48 days or weeks or any time within a range defined by any two of the aforementioned times, for example, 1 day to 48 weeks, 1-48 days, 1-48 weeks, or 10-30 days.

After administration of the dual-specific bifunctional polypeptide, polynucleotide, or pharmaceutical composition, the patient exhibits a prolongment of progression-free survival (PFS), a prolongment of overall survival (OS), a reduction in cancer burden (e.g., tumor size and number), or an amelioration of symptoms such as symptoms of pain, discomfort, or inflammation.

In some embodiments, the dual-specific bifunctional polypeptide, polynucleotide, or pharmaceutical composition is administered as a supplement to a standard of care therapy for a cancer. In some embodiments, the standard of care treatment may include but is not limited to surgery, radiation, chemotherapy, targeted therapy, or immunotherapy. For example, the dual-specific bifunctional polypeptide, polynucleotide, or pharmaceutical composition may be administered in combination with anti-PD-1, anti-PD-L1, anti-CTLA4, anti-LAG3, or adoptive cell therapy. In some embodiments, the combination of the standard of care therapy and the dual-specific bifunctional polypeptide, polynucleotide, or pharmaceutical composition has a greater positive effect in reducing cancer burden or ameliorating symptoms compared to the standard of care therapy alone.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

### REFERENCES

Amaro et al. An Intrabody Drug (rAAV6-INT41) Reduces the Binding of N-Terminal Huntingtin Fragment(s) to DNA to Basal Levels in PC12 Cells and Delays Cognitive Loss in the R6/2 Animal Model. J. Neurodegener. Dis. (2016) 7120753.
Baltz et al. Design and Functional Characterization of Synthetic E3 Ubiquitin Ligases for Targeted Protein Depletion. Curr. Protoc. Chem. Biol. (2018) 10(1):72-90.
Bandukwala et al. Structure of a domain-swapped FOXP3 dimer on DNA and its function in regulatory T cells. Immunity (2011) 34(4):479-91,
Chatterjee et al. Targeted intracellular degradation of SARS-CoV-2 via computationally optimized peptide fusions. Commun Biol. (2020) 3(1):715.
Chen et al. The ubiquitin ligase Stub1 negatively modulates regulatory T cell suppressive activity by promoting degradation of the transcription factor Foxp3. Immunity (2013) 39(2):272-85.
Choo & Zhang. Detection of protein ubiquitination. J Vis. Exp. (2009) 30:1293.
Colby et al. Potent inhibition of huntingtin aggregation and cytotoxicity by a disulfide bond-free single-domain intracellular antibody. PNAS (2004) 101(51):17616-21.
Dixon et al. NanoLuc Complementation Reporter Optimized for Accurate Measurement of Protein Interactions in Cells. ACS Chem Biol. (2016) 11(2):400-8.
Franklin et al. A High-Throughput Assay for Monitoring Ubiquitination in Real Time. Front. Chem. (2019) 7:816.
Fujiwara et al. A single-chain antibody/epitope system for functional analysis of protein-protein interactions. Biochemistry (2002) 41(42):12729-38.
Graham et al. Tof4 can specifically recognize B-catenin using alternate conformations. Nat Struct Biol. (2001) 8(12):1048-52.
Hatakeyama et al. Targeted destruction of c-Myc by an engineered ubiquitin ligase suppresses cell transformation and tumor formation. Cancer Res. (2005) 65(17):7874-9.
Liao et al. A PROTAC peptide induces durable β-catenin degradation and suppresses Wnt-dependent intestinal cancer. Cell Discov. (2020) 6:35.
Lim et al. bioPROTACs as versatile modulators of intracellular therapeutic targets including proliferating cell nuclear antigen (PCNA). PNAS (2020) 117(11):5791-5800.
Lu et al. Discovery of a Keap1-dependent peptide PROTAC to knockdown Tau by ubiquitination-proteasome degradation pathway. Eur. J. Med. Chem. (2018) 146:251-259.
Ludwicki et al. Broad-Spectrum Proteome Editing with an Engineered Bacterial Ubiquitin Ligase Mimic. ACS Cent. Sci. (2019) 5(5):852-866.
Pan et al. A recombinant chimeric protein specifically induces mutant KRAS degradation and potently inhibits pancreatic tumor growth. Oncotarget (2016) 7(28):44299-44309.
Portnoff et al. Ubiquibodies, synthetic E3 ubiquitin ligases endowed with unnatural substrate specificity for targeted protein silencing. J. Biol. Chem. (2014) 289(11):7844-55.
Riching et al. Quantitative Live-Cell Kinetic Degradation and Mechanistic Profiling of PROTAC Mode of Action. ACS Chem Biol. (2018) 13(9):2758-2770.
Samarasinghe et al. OligoTRAFTACs: A Generalizable Method for Transcription Factor Degradation. bioRxiv (2021). Doi:10.1101/2021.12.20.473482.
Soucek et al. Design and properties of a Myc derivative that efficiently homodimerizes. Oncogene (1998) 17(19):2463-72.
Stanley et al. Structural Insight into the Human Immunodeficiency Virus Vif SOCS Box and Its Role in Human E3 Ubiquitin Ligase Assembly. J Virol. (2008) 82(17):8656-8663.
Stephens et al. Engineering Single Pan-Specific Ubiquitibodies for Targeted Degradation of All Forms of Endogenous ERK Protein Kinase. ACS Synth Biol. (2021) 10(9):2396-2408.
Traenkle et al. Monitoring interactions and dynamics of endogenous beta-catenin with intracellular nanobodies in living cells. Mol Cell Proteomics (2015) 14(3):7070-23.
Udeshi et al. Large-scale identification of ubiquitination sites by mass spectrometry. Nat Protoc. (2013) 8(10):1950-60.
Wang et al. The state of the art of PROTAC technologies for drug discovery. Eur J. Med. Chem. (2022) 235:114290.
Welcker et al. The Fbw7 tumor suppressor regulates glycogen synthase kinase 3 phosphorylation-dependent c-Myc protein degradation. PNAS (2004) 101(24):9085-90.
Wu et al. The DDB1-DCAF1-Vpr-UNG2 crystal structure reveals how HIV-1 Vpr steers human UNG2 towards destruction. Nat Struct Mol. Biol. (2016) 23(10):933-940.
Yang et al. Circular mRNA encoded PROTAC (RiboPROTAC) as a new platform for the degradation of intracellular therapeutic targets. BioRxiv (2022). Doi: 10.1101/2022.04.22.489232.
WO 2014/193632.

1. A dual-specific bifunctional polypeptide that promotes proteasome-mediated degradation of at least one target protein, comprising:
g) a first targeting moiety that is capable of binding to at least one target protein, optionally an intracellular target protein,
h) a second targeting moiety that is capable of binding to at least one target protein, optionally an intracellular target protein,
i) a ubiquitin-proteasome system recruiting domain (URD),
j) optionally a first linker peptide, wherein the first linker peptide is positioned between the first targeting moiety and the URD or between the second targeting moiety and the URD,
k) optionally a second linker peptide, wherein the second linker peptide is positioned between the second targeting moiety and the URD or between the first targeting moiety and the second targeting moiety, and
l) optionally one or more localization peptide sequences,
wherein proximity of the dual-specific bifunctional polypeptide to at least one target protein through binding of the first targeting moiety and/or the second targeting moiety induces ubiquitination of the at least one target protein via the URD, thereby promoting proteasome-mediated degradation of the at least one target protein.

2-164. (canceled)
